(19) United States
(12) Patent Application Publication
(10) Pub. No.: US 2024/0299584 A1
(43) Pub. Date: Sep. 12, 2024

(54) NON-CODING RNA PROTECTING AGAINST HEART FAILURE

(71) Applicant: MEDIZINISCHE HOCHSCHULE HANNOVER, Hannover (DE)

(72) Inventors: Thomas Thum, Hannover (GB); Dongchao Lu, Hannover (GB); Christian Bär, Hannover (GB)

(21) Appl. No.: 17/768,778

(22) PCT Filed: Oct. 15, 2020
(86) PCT No.: PCT/EP2020/079022

§ 371 Date: Apr. 13, 2022

(30) Foreign Application Priority Data
Oct. 16, 2019 (GB) EP19203627.5

(51) Int. Cl. A61K 48/00; A61P 9/10; C12N 15/113; C12N 15/86; C12Q 1/686; C12Q 1/6883

(52) U.S. Cl. CPC A61K 48/0058; A61P 9/10; C12N 15/113; C12N 15/86; C12Q 1/686; C12Q 1/6883; C12N 2310/532; C12N 2750/14143

(57) ABSTRACT

The present invention relates to a pharmaceutical composition comprising a compound promoting the expression and/or the activity of the non-coding RNA (ncRNA) of SEQ ID NO: 1 or a sequence being at least 85% identical thereto.

The present invention relates to a pharmaceutical composition comprising a compound promoting the expression and/or the activity of the non-coding RNA (ncRNA) of SEQ ID NO: 1 or a sequence being at least 85% identical thereto.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Circular RNAs are a novel class of non-coding RNAs (ncRNA) which are characterized by covalently linked ends, thus forming closed circles³. Although a few circular RNAs were identified as abnormally spliced transcripts around 20 years ago⁴, recent studies revealed that thousands of genes can be spliced in a non-canonical fashion and produce circular RNAs⁵. These molecules were found highly abundant, stable and often conserved in mammalian and other species^{6, 7}. To date only a few mechanisms of circular RNA have been suggested. Circular RNA Circ-CDR1as has been reported as a molecular sponge for microRNA miR-7 in the brain⁸. Similarly, circ-Hipk3 can trap multiple microRNAs in cancer, such as miR-124, miR-193, miR-379 etc.⁹ Circular RNAs may also interact with proteins; for example, circ-Foxo3 can block the cell-cycle through binding the CDK protein¹⁰. Most circular RNAs originate through the aberrant splicing of exons of protein coding genes, but can also arise from genes of long non-coding RNAs. Circular RNAs are generally non-coding, although recently, circ-ZNF609 was shown to be translated¹¹. The mechanisms of circular RNA in cardiac disease are poorly understood and await characterization.

Heart failure is one of the leading causes of death in the world, as pathological hypertrophy or myocardial infraction causes cardiac remodeling that can ultimately progress to heart failure^{1, 2}. Another critical issue is heart failure caused by the toxicity of many anti-cancer drugs. With better anti-cancer regimens and improved cancer survival rates, the incidence of anti-cancer treatment induced heart failure is also steeply increasing (see Chatterjee et al. (2019), Am J Physiol Heart Circ Physiol.; 316(1):H160-H168. It is therefore necessary to explore novel therapeutic targets for a variety of cardiovascular diseases. This need is addressed by the present invention.

The present invention therefore relates in a first aspect to a pharmaceutical composition comprising a compound promoting the expression and/or the activity of the non-coding RNA (ncRNA) of SEQ ID NO: 1 or a sequence being at least 85% identical thereto.

The present invention likewise relates to a compound promoting the expression and/or the activity of the non-coding RNA (ncRNA) of SEQ ID NO: 1 or a sequence being at least 85% identical thereto for use as a medicament.

In accordance with the present invention, the term “pharmaceutical composition” relates to a composition for administration to a patient, preferably a human patient. The pharmaceutical composition of the invention comprises the compounds recited above. It may, optionally, comprise further molecules capable of altering the characteristics of the compounds of the invention thereby, for example, stabilizing, modulating and/or activating their function. The composition may be in solid, liquid or gaseous form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier or excipient. By “pharmaceutically acceptable carrier or excipient” is meant a non-toxic solid, semisolid or liquid filler, diluent, encapsulating material or formulation auxiliary of any type (see also Handbook of Pharmaceutical Excipients 6ed. 2010, Published by the Pharmaceutical Press). Examples of suitable pharmaceutical carriers and excipients are well known in the art and include phosphate buffered saline solutions, water, emulsions, such as oil/water emulsions, various types of wetting agents, sterile solutions, organic solvents including DMSO etc. Compositions comprising such carriers or excipients can be formulated by well known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the clinician or physician. Generally, the regimen as a regular administration of the pharmaceutical composition should be in the range of 1 μg to 5 g units per day. However, a more preferred dosage might be in the range of 0.01 mg to 100 mg, even more preferably 0.01 mg to 50 mg and most preferably 0.01 mg to 10 mg per day.

Furthermore, if for example said compound is an nucleic acid sequence, such as an siRNA, the total pharmaceutically effective amount of pharmaceutical composition administered will typically be less than about 75 mg per kg of body weight, such as for example less than about 70, 60, 50, 40, 30, 20, 10, 5, 2, 1, 0.5, 0.1, 0.05, 0.01, 0.005, 0.001, or 0.0005 mg per kg of body weight. More preferably, the amount will be less than 2000 nmol of nucleic acid sequence (e.g., about 4.4×1016 copies) per kg of body weight, such as for example less than 1500, 750, 300, 150, 75, 15, 7.5, 1.5, 0.75, 0.15, 0.075, 0.015, 0.0075, 0.0015, 0.00075 or 0.00015 nmol of siRNA agent per kg of body weight.

The length of treatment needed to observe changes and the interval following treatment for responses to occur vary depending on the desired effect. The particular amounts may be determined by conventional tests which are well known to the person skilled in the art.

The pharmaceutical composition may be administered, for example, orally, parenterally, such as subcutaneously, intravenously, intramuscularly, intraperitoneally, intrathecally, transdermally, transmucosally, subdurally, locally or topically via iontopheresis, sublingually, by inhalation spray, aerosol or rectally and the like in dosage unit formulations optionally comprising conventional pharmaceutically acceptable carriers or excipients as described herein above.

The term “ncRNA” or “non-coding RNA” as used herein designates a functional RNA molecule that is not translated into a protein. The RNA of SEQ ID NO: 1 is not translated into protein and therefore an ncRNA.

The sequence being at least 85% identical to SEQ ID NO: 1 as disclosed herein is with increasing preference at least 90%, at least 95%, at least 98%, and at least 99% identical thereto. Means and methods for determining sequence identity are known in the art. Preferably, the BLAST (Basic Local Alignment Search Tool) program is used for determining the sequence identities as referred to herein. Particularly preferred examples of sequences being at least 85% identical to SEQ ID NO: 1 are SEQ ID NOs 2 and 3. SEQ ID NOs 2 and 3 are the orthlogous sequences of the human SEQ ID NO: 1 from mouse and rat, respectively. SEQ ID NOs 2 and 3 share 85.9% and 86.4% sequence identity with SEQ ID NO: 1, respectively.

The compound of the invention may be formulated as vesicles, such as liposomes. Liposomes have attracted great interest because of their specificity and the duration of action they offer from the standpoint of drug delivery. Liposomal delivery systems have been used to effectively deliver nucleic acids, such as siRNA in vivo into cells (Zimmermann et al. (2006) Nature, 441:111-114). Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are phagocytosed by macrophages and other cells in vivo.

A compound promoting the expression of the ncRNA of SEQ ID NO: 1 may be any compound enhancing or upregulating the transcription of said ncRNA. Non-limiting examples of such compounds are transcription factors enhancing the transcription of the genes encoding the ncRNA or a small molecule enhancing the expression of the ncRNA. A transcription factor is a protein binding to specific DNA sequences, thereby controlling the transcription of genetic information from DNA to RNA. A small molecule is a low molecular weight compound which is by definition not a polymer. A particular preferred example is the exon splicing enhancer Brca1 (BReast CAncer 1, early-onset), noting that Brca1 apparently directly regulates the expression of the ncRNA of SEQ ID NO: 1.

A compound promoting the activity of the ncRNA of SEQ ID NO: 1 may be any compound which causes that said ncRNA effectively performs its function in a cell. Hence, in the simplest form such a compound may be the recombinantly produced or isolated ncRNA or any precursor or fragment thereof. In this embodiment the administration of a recombinantly produced or isolated ncRNA increases the concentration of the ncRNA in the subject to be treated. This higher concentration promotes the overall activity of the respective ncRNA in the subject. The fragments have to retain or essentially retain the function of the full-length ncRNA. Hence, the fragments have to be functional fragments. Such a compound may also be a vector or host being capable of producing such the ncRNAs. Also orthologous or homologous sequences of the ncRNA of SEQ ID NO: 1 from different species including precursors or functional fragments thereof may be used. In this regard, preferred homologous sequences of the human ncRNA of SEQ ID NO: 1 are the respective mouse homolog of SEQ ID NO: 2 or the respective rat homolog of SEQ ID NO: 3. Alternatively, such a compound may be a compound maintaining or even enhancing the activity of the ncRNA by either directly or indirectly interacting with the ncRNA. In this respect it is to be understood that compound promoting the expression and/or the activity of the non-coding RNA (ncRNA) of SEQ ID NO: 1 can also be a compound maintaining the activity of the ncRNA. For instance, such a compound may prevent the ncRNA from degeneration by RNases or may be an interaction partner, such as another ncRNA, which binds to and promotes the activity of the ncRNA of SEQ ID NO 1. Further examples of compounds will be further detailed herein below.

The efficiency of a compound to promote the expression and/or the activity of the ncRNA of SEQ ID NO: 1 can be quantified by methods comparing the level of expression and/or activity of the ncRNA of SEQ ID NO: 1 in the presence of a compound promoting the expression and/or activity of the ncRNA, such as a transcription factor, to that in the absence of said compound. For example, as an activity measure the change in amount of ncRNA formed may be used.

The method is preferably effected in high-throughput format in order to test the efficiency of several inhibiting compound simultaneously. High-throughput assays, independent of being biochemical, cellular or other assays, generally may be performed in wells of microtiter plates, wherein each plate may contain 96, 384 or 1536 wells. Handling of the plates, including incubation at temperatures other than ambient temperature, and bringing into contact of test compounds with the assay mixture is preferably affected by one or more computer-controlled robotic systems including pipetting devices. In case large libraries of test compounds are to be screened and/or screening is to be effected within a short time, mixtures of, for example 10, 20, 30, 40, 50 or 100 test compounds may be added to each well. In case a well exhibits the expected activity, said mixture of test compounds may be de-convoluted to identify the one or more test compounds in said mixture giving rise to said activity.

It can be taken from the appended examples that the ncRNA of SEQ ID NO: 1 (also referred to herein as “circ-Insr” or “circINSR”) was surprisingly identified as being an evolutionary highly conserved circular RNA that protects against cardiac vascular disease or a metabolic disorder. In more detail, the examples herein below show that the ncRNA of SEQ ID NO: 1 is capable of restoring or maintaining mitchondrial function in cardiac vascular diseases (such as heart failure and cardiac ischemia-reperfusion injury) or a metabolic disorders being associated with mitochondrial dysfunction. It is demonstrated that circ-Insr is downregulated in diseased heart. Knockdown of circ-Insr in cardiomyocyte-like HL-1 cells increases the rate of apoptosis after treatment with doxorubicin, while AAV6-based and plasmid-based overexpression of circ-Insr is sufficient to rescue doxorubicin-induced cardiomyocyte apoptosis in vitro in HL-1 cells and human stem cell-derived cardiomyocytes. Moreover, in a mouse model of chronic doxorubicin-induced cardiotoxicity, AAV9-mediated overexpression of circ-Insr protects against cardiac apoptosis, atrophy and improves the heart function after doxorubicin treatment. All this data show that promoting the expression and/or activity of the ncRNA of SEQ ID NO: 1 is of medical value, in particular for the treatment and prevention of heart failure. In addition, it is shown in the appended examples that the ncRNA of SEQ ID NO: 1 is capable of maintaining and/or rescuing the metabolic activity and in particular the mitochondrial function of cells, such as cardiomyocytes. For instance, the expression of the ncRNA of SEQ ID NO: 1 was found to significantly decrease the infarct region of IR injury heart. Hence, the ncRNA of SEQ ID NO: 1 has a protective role and can be used to treat and prevent cardiac vascular disease or a metabolic disorder.

Without wishing to be bound by this theory it is believed that the metabolic protective effect of the ncRNA of SEQ ID NO: 1 is mediated by the binding of the ncRNA to the protein SSBP1 (single-stranded DNA-binding protein 1). The SSBP 1 gene is a housekeeping gene involved in mitochondrial biogenesis. SSBP 1 is known to act as a homotetramer to stabilize the displaced single strand of the normal and expanded displacement loop (D loop) during mtDNA replication, thus preventing formation of secondary single-stranded DNA structures, which could stop the gamma-DNA polymerase.

The present invention relates in a second aspect to a compound promoting the expression and/or the activity of the ncRNA of SEQ ID NO: 1 or a sequence being at least 85% identical thereto for use in treating or preventing a cardiac vascular disease or a metabolic disorder, wherein the cardiac vascular disease is preferably heart failure or cardiac ischemia-reperfusion injury, and wherein the heart failure is preferably anthracycline-induced heart failure, more preferably doxorubicin-, epirubicin- or daunorubicin-induced heart failure and most preferably doxorubicin-induced heart failure.

The present invention also relates to a method for treating or preventing a cardiac vascular disease or a metabolic disorder by administering an effective amount of a compound promoting the expression and/or the activity of the ncRNA of SEQ ID NO: 1 or a sequence being at least 85% identical thereto in a subject in need thereof, wherein the cardiac vascular disease is preferably heart failure or cardiac ischemia-reperfusion injury, and wherein the heart failure is preferably anthracycline-induced heart failure, more preferably doxorubicin-, epirubicin- or daunorubicin-induced heart failure and most preferably doxorubicin-induced heart failure.

The subject as referred to herein is preferably human. In connection with the second aspect of the invention as far as pertaining to the treatment or prevention of anthracycline-induced heart-failure, preferably doxorubicin-induced heart-failure, the human subject has preferably cancer and is also treated with anthracycline, preferably doxorubicin at the same time, before or after being treated with a compound promoting the expression and/or the activity of the ncRNA of SEQ ID NO: 1 or a sequence being at least 85% identical thereto.

A cardiac vascular disease (CVD) is a condition affecting the heart or blood vessels. A CVD is usually associated with a build-up of fatty deposits inside the arteries (atherosclerosis) and an increased risk of blood clots. The CVD is preferably a CVD being associated with mitochondrial dysfunction of cardiomyocytes since it is shown in the examples herein below that the ncRNA of SEQ ID NO: 1 is capable of maintaining or improving the mitochondrial function in cardiomyocytes.

A metabolic disorder is a disorder being characterized by abnormal chemical reactions in the body of subject which disrupt metabolism. Metabolism is the process within the body of the subject whereby energy is generated from the nutrition being consumed by the subject. The metabolic disorder is preferably a mitochondrial disease since it is shown in the examples herein below that the ncRNA of SEQ ID NO: 1 is capable of maintaining or improving the mitochondrial function of cells. Mitochondrial diseases are a group of metabolic disorders being characterized by the impairment of mitochondrial function. The most prominent function of mitochondria is to produce the energy currency of the cell, ATP (i.e., phosphorylation of ADP), through respiration, and to regulate cellular metabolism. The mitochondrial disease is preferably associated with mitochondrial dysfunction of cardiomyocytes.

Mitochondrial dysfunction of cells as well as maintaining or improving the mitochondrial function of cells may be monitored by methods well-known in the art which are in part also described in the appended examples. Non-limiting examples are methods for determining a metabolism of the cell, such as basal respiration, maximal respiration, spare respiratory capacity or ATP production. Among this list maximal respiration and spare respiratory capacity are preferred. Also the expression of mitochondrial biogenesis-related genes (e.g. Pgc1-α, Nrf2a, Nrf2b, Tfb1m, Tfb2m and/or SSBP1) or or of mtDNA may be monitored. As a further example the mitochondrial membrane potential may be measured, noting that the impairment of the mitochondrial membrane potential is an indication of mitochondrial dysfunction. Yet further the percentage of cell undergoing mitochondrial fission may be monitored, noting that mitochondrial fission is an indication of mitochondrial dysfunction.

Heart-failure is a chronic, generally progressive condition in which the heart muscle is unable to pump enough blood to meet the body's needs for blood and oxygen.

In order to compensate the lack of oxygen the heart often enlarges. In more detail, the heart stretches to contract more strongly and keep up with the demand to pump more blood. Over time this causes the heart to become enlarged. The heart may also build more muscle mass. The increase in muscle mass occurs because the contracting cells of the heart get bigger thereby making the heart pump stronger, at least initially. Also the heart may pump faster, thereby increasing the heart's output.

Also the body may try to compensate heart-failure. For instance, the blood vessels may narrow to keep blood pressure up, trying to make up for the heart's loss of power. The body may divert blood away from less important tissues and organs (like the kidneys) to the important tissues heart and brain.

These temporary measures mask the problem of heart failure, but they do not solve it. Heart failure generally continues and worsens until these compensating processes no longer work.

Anthracyclines, including doxorubicin, epirubicin and daunorubicin, are some of the most efficacious anticancer drugs available. Anthracyclines are a class of drugs used in cancer chemotherapy that are extracted from Streptomyces bacteria. These compounds are used to treat many cancers, including leukemias, lymphomas, breast, stomach, uterine, ovarian, bladder cancer, and lung cancers. Doxorubicin, also known as Adriamycin® or Rubex®, is an anthracycline antibiotic that was discovered from a mutated strain of Streptomyces peucetius. Doxorubicin operates on several levels by different molecular mechanisms including an interaction with iron, upsetting calcium homeostasis, altering the activity of intracellular or intra-mitochondrial oxidant enzymes, and binding to topoisomerases promoting their dysfunction. While doxorubicin is the most popular anthracycline, in some chemotherapy regimens epirubicin is favoured over doxorubicin, as epirubicin appears to cause fewer side-effects in these chemotherapy regimens. Epirubicin has a different spatial orientation of the hydroxyl group at the 4′ carbon of the sugar—it has the opposite chirality-which may account for its faster elimination and reduced toxicity. Daunorubicin may be used as as the starting material for semi-synthetic manufacturing of doxorubicin and epirubicin. Daunorubicin is generally only administered by intravenous infusion since other administration routes might cause extensive cell or tissue necrosis.

Anthracyclines have meanwhile been used for over 3 decades despite numerous side effects. The studies of survivors 4 to 20 years after doxorubicin treatment observed significant decreases in fractional shortening and ejection fractions, and that was dependent upon the cumulative dose. Analysis of heart transplantation patients found doxorubicin as the underlying cause in 2-3% of all cases. Hence, anthracyclines and in particular doxorubicin are cardiotoxic and led to heart failure. The increased risk of heart failure from doxorubicin can manifest acutely during treatment or chronically weeks to years after the treatment has ceased (Mirty and Edwards (2016), Int J Cardiol Heart Vasc. 2016 March; 10: 17-24).

Cardiac ischemia-reperfusion injury (or reperfusion injury or cardiac IRI) is the heart damage caused when blood supply returns to the heart (re-+perfusion) after a period of ischemia or lack of oxygen (anoxia or hypoxia). The absence of oxygen and nutrients from blood during the ischemic period creates a condition in which the restoration of circulation results in inflammation and oxidative damage through the induction of oxidative stress rather than (or along with) restoration of normal function.

In accordance with a preferred aspect of the first and second aspect of the invention the compound is (a) a nucleic acid sequence which comprises or consists of the nucleic acid sequence of the ncRNA of SEQ ID NO: 1 or an nucleic acid sequence which is at least 70% identical thereto, (b) an expression vector expressing the nucleic acid sequence as defined in (a), preferably under the control of a heart-specific promoter, or (c) a host comprising the expression vector of (b).

The term “nucleic acid sequence” or “nucleotide sequence”, in accordance with the present invention, includes DNA, such as cDNA or, in a preferred embodiment genomic DNA, and RNA. It is understood that the term “RNA” as used herein is non-coding RNA. The term “nucleic acid sequence” is interchangeably used in accordance with the invention with the term “polynucleotide”.

The nucleic acid sequence according to item (a) of this preferred embodiment may be a recombinantly produced or isolated ncRNA of SEQ ID NO: 1, any precursor thereof or any fragment thereof as long as a sequence identity of at least 70% over the entire length of an ncRNA of SEQ ID NO: 1. Also orthologous or homologous sequences of the ncRNA selected from different species including precursor or a functional fragment thereof may be used. Preferably the respective mouse homologs of SEQ ID NO: 2 or rat homolog of SEQ ID NO: 3 is used. The fragments have to retain or essentially retain the function of the full-length ncRNA. Hence, the fragments have to be functional fragments.

The sequence identity of the nucleic acid sequence according to item (a) to the ncRNA of SEQ ID NO: 1 is with increasing preference at least 75%, at least 80%, at least 85%, at least 90%, at least 95%, at least 98%, and at least 99%.

In accordance with items (b) and (c) of the above preferred embodiment such a compound may also be an expression vector or host being capable of producing an nucleic acid sequence as defined in item (a).

An expression vector may be a plasmid that is used to introduce a specific transcript into a target cell. Once the expression vector is inside the cell, the transcript that is encoded by the gene is produced by the cellular-transcription machinery. The plasmid is in general engineered to contain regulatory sequences that act as enhancer and/or promoter regions and lead to efficient transcription of the transcript. In accordance with the present invention the expression vector preferably contains a heart-specific promoter. Heart-specific promoters are known in the art, for example, from Boecker at al. (2004), Mol Imagin.; 3(2):69-75. For instance, the CMW-promoter ensures preferential expression in the heart. This ensures that the nucleic acid sequence is only expressed in the heart and may avoid potential unwanted side effects by expression in other organs.

Non-limiting examples of expression vectors include prokaryotic plasmid vectors, such as the pUC-series, pBluescript (Stratagene), the pET-series of expression vectors (Novagen) or pCRTOPO (Invitrogen) and vectors compatible with an expression in mammalian cells like PREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, pLXIN, pSIR (Clontech), pIRES-EGFP (Clontech), PEAK-10 (Edge Biosystems) pTriEx-Hygro (Novagen) and pCINeo (Promega). Examples for plasmid vectors suitable for Pichia pastoris comprise e.g. the plasmids pAO815, pPIC9K and pPIC3.5K (all Invitrogen). For the formulation of a pharmaceutical composition a suitable vector is selected in accordance with good manufacturing practice. Such vectors are known in the art, for example, from Ausubel et al, Hum Gene Ther. 2011 April; 22(4):489-97 or Allay et al., Hum Gene Ther. May 2011; 22(5): 595-604.

A typical mammalian expression vector contains the promoter element, which mediates the initiation of transcription of RNA, the transcript sequence, and signals required for the termination of transcription and optionally polyadenylation of the transcript. Moreover, elements such as origin of replication, drug resistance gene, regulators (as part of an inducible promoter) may also be included. The lac promoter is a typical inducible promoter, useful for prokaryotic cells, which can be induced using the lactose analogue isopropylthiol-b-D-galactoside. (“IPTG”). Additional elements might include enhancers, Kozak sequences and intervening sequences flanked by donor and acceptor sites for RNA splicing. Highly efficient transcription can be achieved with the early and late promoters from SV40, the long terminal repeats (LTRs) from retroviruses, e.g., RSV, HTLVI, HIVI, and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12 MI (ATCC 67109). Alternatively, the recombinant transcript can be expressed in stable cell lines that contain the gene construct integrated into a chromosome. The co-transfection with a selectable marker such as dhfr, gpt, neomycin, hygromycin allows the identification and isolation of the transfected cells. The transfected nucleic acid can also be amplified to express large amounts of the encoded transcript. The DHFR (dihydrofolate reductase) marker is useful to develop cell lines that carry several hundred or even several thousand copies of the gene of interest. Another useful selection marker is the enzyme glutamine synthase (GS) (Murphy et al. 1991, Biochem J. 227:277-279; Bebbington et al. 1992, Bio/Technology 10:169-175). Using these markers, the mammalian cells are grown in selective medium and the cells with the highest resistance are selected. As indicated above, the expression vectors will preferably include at least one selectable marker.

Such markers include dihydrofolate reductase, G418 or neomycin resistance for eukaryotic cell culture and tetracycline, kanamycin or ampicillin resistance genes for culturing in E. coli and other bacteria. For vector modification techniques, see Sambrook and Russel (2001), Molecular Cloning: A Laboratory Manual, 3 Vol. Generally, vectors can contain one or more origins of replication (ori) and inheritance systems for cloning or expression, one or more markers for selection in the host, e.g., antibiotic resistance, and one or more expression cassettes. Suitable origins of replication (ori) include, for example, the Col E1, the SV40 viral and the M 13 origins of replication.

The transcript sequences inserted in the vector can e.g. be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression in prokaryotes or eukaryotic cells are well known to those skilled in the art. These elements comprise regulatory sequences ensuring the initiation of the transcription (e.g., translation initiation codon, promoters, enhancers, and/or insulators), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers, and/or naturally-associated or heterologous promoter regions. Preferably, the nucleotide sequence as defined in item (a) of the above preferred embodiment of the invention is operatively linked to such expression control sequences allowing expression in prokaryotic or eukaryotic cells.

The host may be a prokaryotic or eukaryotic cell. A suitable eukaryotic host may be a mammalian cell, an amphibian cell, a fish cell, an insect cell, a fungal cell or a plant cell. Representative examples of bacterial cells are E. coli, Streptomyces and Salmonella typhimurium cells; of fungal cells are yeast cells; and of insect cells are Drosophila S2 and Spodoptera Sf9 cells. It is preferred that the cell is a mammalian cell such as a human cell. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells. The cell may be a part of a cell line, preferably a human cell line. Appropriate culture mediums and conditions for the above-described host cells are known in the art. The host is preferably a host cell and more preferably an isolated host cell. The host is also preferably a non-human host.

In accordance with another preferred aspect of the first and second aspect of the invention the nucleic acid sequence is a circular nucleic acid sequence.

The ncRNA of SEQ ID NO: 1 naturally occurs as a circular RNA. Circular RNAs (circRNAs) are a subclass of non-coding RNAs that lack free 3′ and 5′ ends and, thus, exist as continuous loop RNAs. While it is demonstrated in the examples herein below that the ncRNA of SEQ ID NO: 1 is also pharmaceutically active in its linear form it is preferred that the nucleic acid sequence is a circular nucleic acid sequence. The circular form offers certain advantages, e.g. better protection against degradation in vivo by exonuclease attack.

In accordance with a more preferred aspect of the first and second aspect of the invention the circular nucleic acid sequence is flanked by short interspersed nucleotide elements (SINEs) mediating the circularisation.

Short interspersed nuclear elements (SINEs) are non-autonomous, non-coding transposable elements (TEs) that are about 100 to 700 base pairs in length. They are a class of retrotransposons, DNA elements that amplify themselves throughout eukaryotic genomes, often through RNA intermediates.

Alu elements, short-interspersed nuclear element of about 300 nucleotides, are the most common SINE in humans, with >1,000,000 copies throughout the genome, which is over 10 percent of the total genome. Alu elements are also employed in the examples since they facilitate circRNA formation and therefore Alu elements are the preferred SINEs to be used in connection with the above more preferred aspect of the first and second aspect of the invention.

Moreover, there are currently three different models on backsplicing; i.e. models on how RNA molecules can form rings in vivo (see Santer et al. (2019), Molecular Therapy, 27(8): 1350-1363). As at the genomic locus encoding SEQ ID NO: 1 ALU element sequences can be found on both sides of the exon, a ALU mediated alternative splicing is the most likely mechanism of circularisation of the circRNA comprising SEQ ID NO: 1 in vivo.

The upstream and downstream ALU elements flanking the genomic locus encoding SEQ ID NO: 1 are shown in SEQ ID NO: 4 and 5. The entire upstream and downstream flanking regions are shown in SEQ ID NO: 5 and 7 and the the genomic locus encoding SEQ ID NO: 1 is shown in SEQ ID NO: 8.

The corresponding upstream and downstream SINEs flanking the genomic locus encoding SEQ ID NO: 2 and 3 are shown in SEQ ID NO: 9/10 and 14/15, respectively. The entire upstream and downstream flanking regions are shown in SEQ ID NO: 11/12 and 16/17, respectively and the genomic loci encoding SEQ ID NOs 2 and 3 are shown in SEQ ID NOS 13 and 18, respectively.

Hence, preferably the SINEs of SEQ ID NOs 4/5, 9/10 or 14/15 are used for RNA circularisation. Most preferably the ALU elements of SEQ ID NOs 4/5 are used. In this conncetion it is to be understood that the circRNA then comprises the reverse complement of the genomic SINEs, wherein T is replaced by U.

It is generally possible to produce circRNA in vitro by i) lariat-driven circulation that gives rise to circRNAs from lariat intermediate structures, which are produced during exon skipping, or by (ii) intron-pairing driven circularization which is a more direct circRNA formation and involves hybridization of flanking introns, which brings splice sites in close proximity (see Santer et al. (2019), Molecular Therapy, 27(8): 1350-1363).

In accordance with a furthermore preferred aspect of the first and second aspect of the invention the expression vector is an adeno-associated vector, preferably an AAV9 vector.

Adeno-associated virus (AAV) is a non-enveloped virus that can be engineered to deliver DNA to target cells and has attracted a significant amount of attention in the field, especially in clinical-stage experimental therapeutic strategies. The ability to generate recombinant AAV particles lacking any viral genes and containing DNA sequences of interest for various therapeutic applications has thus far proven to be one of the safest strategies for gene therapies (for review, Naso et al. (2017), BioDrugs; 31(4): 317-334.).

AAV9 has a preference for primary cell binding through galactose as a result of unique amino acid differences in its capsid sequence. It has been postulated that this preferential galactose binding could confer AAV9 with the unique ability to cross the blood-brain barrier (BBB) and infect cells of the CNS, including primary neurons. AAV9 has also been shown to be very effective at delivering genes to skeletal and cardiac muscle in various species.

In accordance with another more preferred aspect of the first and second aspect of the invention the heart-specific promoter is a cardiomyocyte specific promoter, preferably the cardiomyocyte specific cTNT promoter.

The cTNT promoter is the promoter of the cardiac troponin T (cTnT). By using eGFP transgene it was shown that AAV9-mediated gene expression from the cTnT promoter is 640-fold greater in the heart muscle cells compared to the next highest tissue (liver) (Prasad et al. (2011), Gene Ther; 18(1): 43-52).

Cardiomyocytes (or cardiac muscle cells) are the muscle cells (myocytes) that make up the cardiac muscle (heart muscle). Each myocardial cell contains myofibrils, which are specialized organelles consisting of long chains of sarcomeres, the fundamental contractile units of muscle cells. Cardiomyocytes show striations similar to those on skeletal muscle cells. Unlike multinucleated skeletal cells, the majority of cardiomyocytes contain only one nucleus, although they may have as many as four. Cardiomyocytes have a high mitochondrial density, which allows them to produce adenosine triphosphate (ATP) quickly, making them highly resistant to fatigue.

In accordance with preferred aspect of the first and second aspect of the invention the compound as defined in (i) is (a) a transcription factor promoting the expression of the ncRNA of SEQ ID NO: 1 or a sequence being at least 85% identical thereto, and/or (b) a small molecule enhancing the expression of the ncRNA of SEQ ID NO: 1 or a sequence being at least 85% identical thereto.

The term “transcription factor” as used herein defines a protein or peptide that binds to specific DNA sequences, thereby controlling the transcription of the genes encoding SEQ ID NO: 1 or a sequence being at least 85% identical thereto. The efficiency of a transcription factor in activating the expression of the ncRNA of SEQ ID NO: 1 or a sequence being at least 85% identical thereto can be quantified by methods comparing the level of the ncRNA in the presence of the transcription factor to that in the absence of the transcription factor. For example, as an activity measure the change in amount of ncRNA formed may be used. Such a method may be effected in high-throughput format in order to test the efficiency of several inhibiting compound simultaneously. High-throughput formats have been further detailed herein above.

The small molecule enhancing the expression of the lncRNA of SEQ ID NO: 1 or a sequence being at least 85% identical thereto is a low molecular weight organic compound which is by definition not a polymer. The small molecule is preferably a molecule that binds with high affinity to the ncRNA of SEQ ID NO: 1 or a sequence being at least 85% identical thereto and in addition enhances the activity of said ncRNA. The upper molecular weight limit for a small molecule is preferably 1500 Da, more preferably 1000 Da and most preferably 800 Da which allows for the possibility to rapidly diffuse across cell membranes so that they can reach intracellular sites of action. Libraries of small organic molecules and high-throughput techniques for screening such libraries with a specific target molecule, in the present case the lncRNA of SEQ ID NO: 1 or a sequence being at least 85% identical thereto, are established in the art.

The present invention relates in a third aspect to a method for diagnosing heart failure or a predisposition to heart failure in a patient, comprising (a) detecting the expression level of the ncRNA of SEQ ID NO: 1 or a sequence being at least 85% identical thereto in a sample obtained from the patient, and (b) comparing the expression level of (a) with the expression level of the ncRNA of SEQ ID NO: 1 or a sequence being at least 85% identical thereto in a control sample obtained from healthy subjects or a predetermined standard from healthy subjects, wherein a greater than 2-fold downregulation of the ncRNA of SEQ ID NO: 1 or a sequence being at least 85% identical thereto is indicative for a heart failure or a predisposition to heart failure in the patient.

The method according to the third aspect of the invention may also encompass detecting and comparing the expression level of one or more ncRNAs being with increased preference at least 90%, at least 92%, at least 94%, at least 96%, at least 98%, at least 99%, and at least 99.5% identical to SEQ ID NO: 1. Means and methods for determining sequence identity are known in the art. Preferably, the BLAST (Basic Local Alignment Search Tool) program is used for determining the sequence identity with regard to the ncRNA of SEQ ID NO: 1. The method according to the third aspect of the invention may furthermore encompass detecting and comparing the expression level of a ncRNAs differing with increasing preference by no more than 10, such as 5, 4, 3, 2 or 1 nucleotide(s) from SEQ ID NO: 1. The nucleotide differences may be the addition, deletion and/or substitution of nucleotide(s). The sequences the expression of which is compared, while being homologous, may also differ from each other with increasing preference by no more than 10, such as 5, 4, 3, 2 or 1 nucleotide(s).

A predisposition to heart failure means that the patient is at risk of developing and preferably will develop a heart failure in the future. In this respect “future” means with increasing preference within 3 years, 2 year and 1 year before.

The term “sample” designates a tissue sample or a body fluid sample. The body fluid sample is preferably selected from blood, serum, plasma, urine, salvia, amniotic fluid, cerebrospinal fluid and lymph. The tissue sample is preferably an organ sample, such as a heart, liver or kidney sample. As far as the method is applied to a body fluid sample it is to be understood that the expression level of an ncRNA corresponds to the concentration of the ncRNA, because ncRNAs are not directly expressed in the body fluid but secreted from the cells, said cells expressing the ncRNAs, into the body fluids.

The “patient” or “subject” referred to herein is preferably human.

The term “detecting the expression level of ncRNA” means determining the amount or yield of the ncRNA. The ncRNAs are initially expressed within a cell. It was found in accordance with the present invention that the ncRNAs of SEQ ID NO: 1 can be detected in the sample of a patient, in particular a heart tissue sample. An ncRNA being “expressed in a sample” is therefore a ncRNA whose expression level can be detected in the sample by means and methods being further detailed herein below. An ncRNA is upregulated in a test sample if the amount or yield of the ncRNA is significantly greater as compared to the amount or yield of the corresponding ncRNA in a control sample. Likewise, an ncRNA is downregulated in a test sample if the amount or yield of the ncRNA is significantly less as compared to the amount or yield of the corresponding ncRNA in a control sample. In this context the term “corresponding ncRNA” implies, for example, that the expression level of the ncRNA of SEQ ID NO: 1 in the test sample is compared to the expression level of the ncRNA of SEQ ID NO: 1 in the control sample.

The expression level in the samples can be quantified by any suitable means and methods available from the art. In general relative and absolute quantification means and methods can be used. In absolute quantification the expression level can be directly quantified in absolute numbers. As well-known in the art, absolute quantification may rely on a predetermined standard curve. In relative quantification the expression level is quantified relative to a reference (such as known control expressions levels). Also in the absence of controls, one can relatively quantify the expression level when comparing e.g. fluorescence intensities.

Methods to assess RNA concentration may, for example, comprise measuring the fluorescence intensity of dyes that bind to nucleic acids and selectively fluoresce when bound. Such methods comprise a reverse transcription reaction and the production of cDNA, wherein the amount of the cDNA is determined thereby indirectly determining the amount of the RNA.

The fluorescent-based method is particularly useful for cases where the RNA concentration is too low to accurately assess some with spectrophotometry and/or in cases where contaminants absorbing at 260 nm make accurate quantification by spectrophotometry difficult or impossible.

When comparing the expression level of the one or more ncRNAs between different samples reliability of the comparison is improved by including an invariant endogenous control (expression of a reference gene) preferably to correct for potential sample to sample variations. Such normalization with respect to an invariant endogenous control is routinely performed in the art. For example, means and methods for expression level normalization, e.g. in real-time RT-PCR (see, for example, Bustin, Journal of Molecular Endocrinology, (2002) 29, 23-39) or micro-array expression analysis (see, for example, Calza and Balwitan, Methods Mol Biol. 2010; 673:37-52) are well-established. Also methods for normalization of the expression levels of small RNA sequences are established (see, for example, Mestdagh et al. (2009) Genome Biol.; 10(6):R64). In case RT-PCR or a micro-array is used to determine the expression levels in accordance with the present invention, the expression levels are preferably normalized to a spiked-in RNA (see, for example, McCormick et al. (2011), Silence, 2:2). Known amounts of a spiked-in RNA are mixed with the sample during preparation. More preferably the RNA is externally spiked-in to plasma and/or serum before the RNA isolation process is carried out, in which case the samples are plasma and/or serum. The spiked-in RNA technology is well-known and commercial kits are available from a number of manufacturers. The spiked-in RNA is preferably a synthetic spiked-in RNA since it is frequently used in the art.

In the examples herein below the primer sequences of SEQ ID NOs 22 to 27 were employed in order to detect the expression level of the ncRNA. These primer sequences hybridize to ncRNA sequences of DSEQ ID NOS 1 to 3 from human, mouse and rat, respectively. For this reason, it is preferred that for the detecting the expression level of the ncRNA of SEQ ID NO: 1 or a sequence being at least 85% identical thereto at least one of primer sequences of SEQ ID NOs 22 to 27 is employed, wherein SEQ ID NOs 22 and 23 are particularly preferred. More preferably one of the primer pairs of SEQ ID NOs 22/23, 24/25 and 26/27 is employed, wherein the primer pair of SEQ ID NOs 22 and 23 is particularly preferred.

The greater than 2-fold downregulation is with increasing preference greater than 3-fold downregulation, greater than 4-fold downregulation, greater than 5-fold downregulation, greater than 6-fold downregulation, greater than 7-fold downregulation and greater than 8-fold downregulation. The higher thresholds for the downregulation is expected to increase the reliability of the method of the third aspect of the invention.

In accordance with a preferred embodiment of the third aspect of the invention said samples are blood samples or blood-derived samples.

The blood-derived sample is preferably plasma or serum.

In accordance with a further preferred embodiment of the third aspect of the invention said samples are a heart tissue samples.

The heart tissue sample comprises preferably muscle cells of the heart.

In accordance with another preferred embodiment of the third aspect of the invention the healthy subjects are at least 3 healthy subjects, preferably at least 5 healthy subjects and most preferably at least 10 healthy subjects.

Also increasing the number of healthy subjects is expected to increase the reliability of the method of the third aspect of the invention since expression differences among individual healthy subjects are balanced.

In accordance with an additional preferred embodiment of the third aspect of the invention the detection of the expression level of the ncRNAs comprises (a) quantitative PCR, preferably quantitative real time PCR, or (b) a template/RNA amplification method followed by determining the expression level of the ncRNA using a fluorescence- or luminescence-based quantification method.

In quantitative PCR (qPCR), the amount of amplified product is linked to fluorescence intensity using a fluorescent reporter molecule. The point at which the fluorescent signal is measured in order to calculate the initial template quantity can either be at the end of the reaction (endpoint semi-quantitative PCR) or while the amplification is still progressing (real-time qPCR).

In endpoint semi-quantitative PCR, fluorescence data are collected after the amplification reaction has been completed, usually after 30-40 cycles, and this final fluorescence is used to back-calculate the amount of template present prior to PCR.

The more sensitive and reproducible method of real-time qPCR measures the fluorescence at each cycle as the amplification progresses. This allows quantification of the template to be based on the fluorescence signal during the exponential phase of amplification, before limiting reagents, accumulation of inhibitors, or inactivation of the polymerase have started to have an effect on the efficiency of amplification. Fluorescence readings at these earlier cycles of the reaction will measure the amplified template quantity where the reaction is much more reproducible from sample to sample than at the endpoint.

A non-limiting example of a template/RNA amplification method followed by determining the expression level of the one or more ncRNAs using a fluorescence- or luminescence-based quantification method is a method combining transcription mediated amplification (TMA) and a hybridization protection assay (HPA). In more detail, such a method may comprise hybridizing one or more oligonucleotides (“capture oligonucleotides”) that are complementary to SEQ ID NO: 1. The hybridized target sequences are then captured onto magnetic microparticles that are separated from the sample in a magnetic field. Wash steps may be utilized to remove extraneous components. Target amplification typically occurs via TMA, which is a transcription-based nucleic acid amplification method that utilizes two enzymes, Moloney murine leukemia virus (MMLV) reverse transcriptase and T7 RNA polymerase. A unique set of primers is used for the target sequence of SEQ ID NO: 1. The reverse transcriptase is used to generate a DNA copy (containing a promoter sequence for T7 RNA polymerase) of the target sequence. T7 RNA polymerase produces multiple copies of RNA amplicon from the DNA copy. Detection of ncRNA expression level is achieved by HPA using single-stranded, chemiluminescent-labeled nucleic acid probes that are complementary to the one or more amplicon. Preferably, distinguishably labelled probes are used for each target amplicon. The labeled nucleic acid probes hybridize specifically to the amplicon. A “selection reagent” then differentiates between hybridized and unhybridized probes by inactivating the label on unhybridized probes. During the detection step, the chemiluminescent signal produced by the hybridized probe is measured in a luminometer and is reported as “Relative Light Units” (RLU), thereby quantifying the ncRNA expression level.

In accordance with a yet further preferred embodiment of the third aspect of the invention the method comprises prior to the detection of the expression level of the ncRNA a pre-amplification step of the RNA within the test patient's sample and/or the control patient's sample.

Performing a pre-amplification step is of particular advantage in case only a low amount of (test and/or control) sample is available. The pre-amplification step allows increasing the amount of RNA within the sample before proceeding to the analysis of the expression level. Means and methods for the pre-amplification of RNA are well known in the art (see, e.g., Vermeulen et al (2009) BMC Res Notes., 2:235). In case both the RNA in the test and control sample is pre-amplified preferably the same method for the pre-amplification step is used such that the relative amount of RNA of the test sample as compared to the control sample is maintained. In case only the RNA of the test or control sample is pre-amplified or the two RNA samples are pre-amplified by different methods, the expression level data may have to be normalized for pre-amplification step; see, e.g. Mestdagh et al. (2009), Genome Biology 2009, 10:R64.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show.

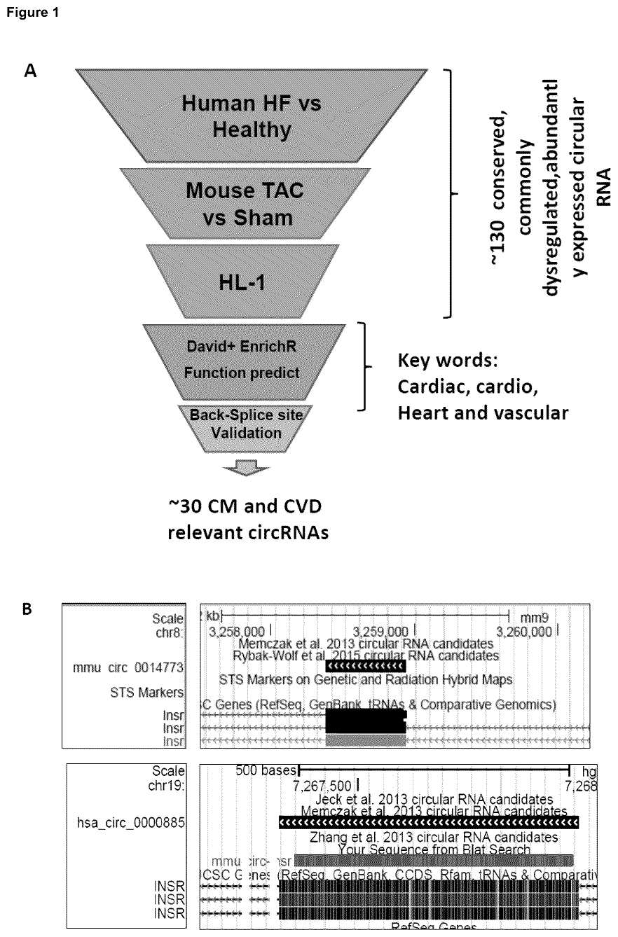

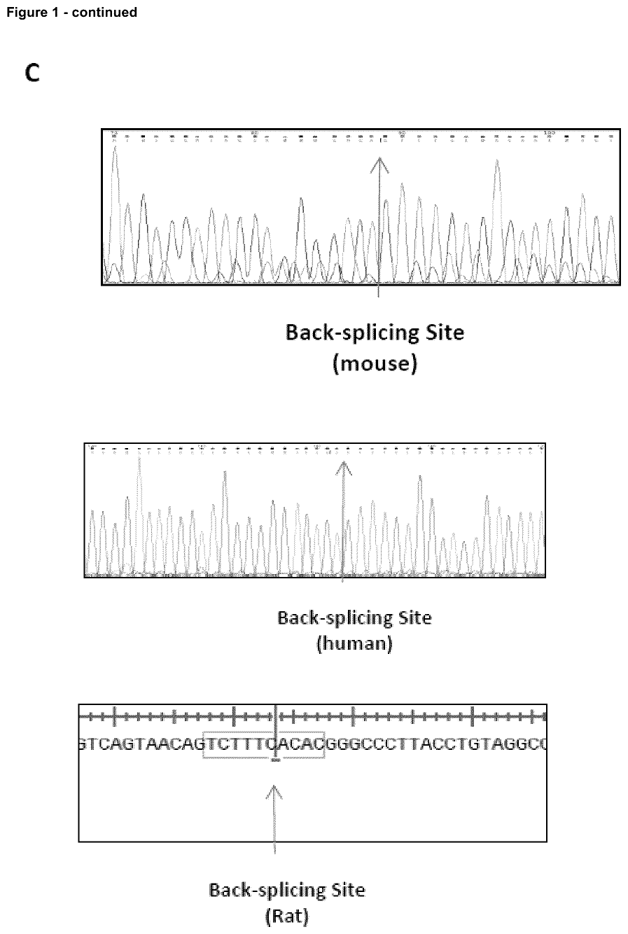

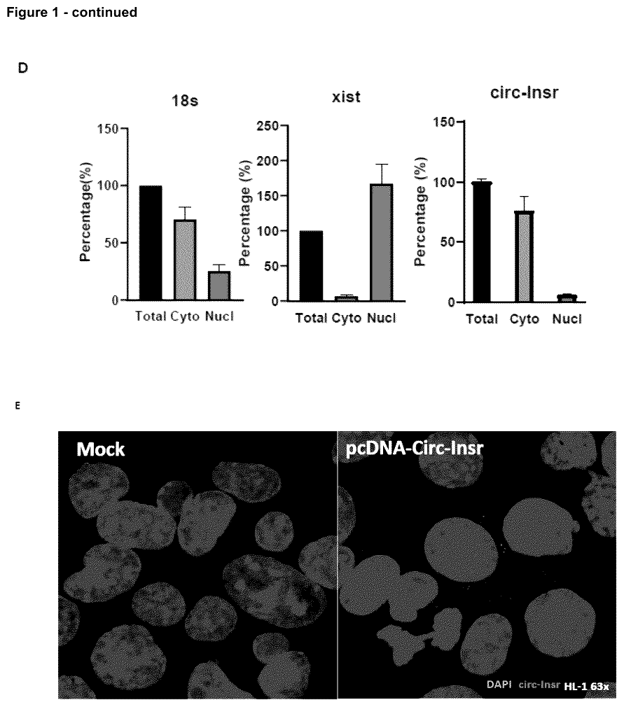

FIG. 1: Bioinformatics and molecular approach to identify conserved circINSR. (A) pipeline to select candidates from RNA-seq data; (B) Genomic locus of circ-Insr in mouse and human represented in UCSC Genome Browser; (C) Sanger sequencing mouse, human and rat circINSR PCR product confirm backsplicing; (D) Circ-Insr expression in HL-1 cells subcellular fraction (n=3); (E) RNA-FISH staining with a circINSR specific probe confirms mainly cytoplasmic localisation (Scale bar, 100 μm).

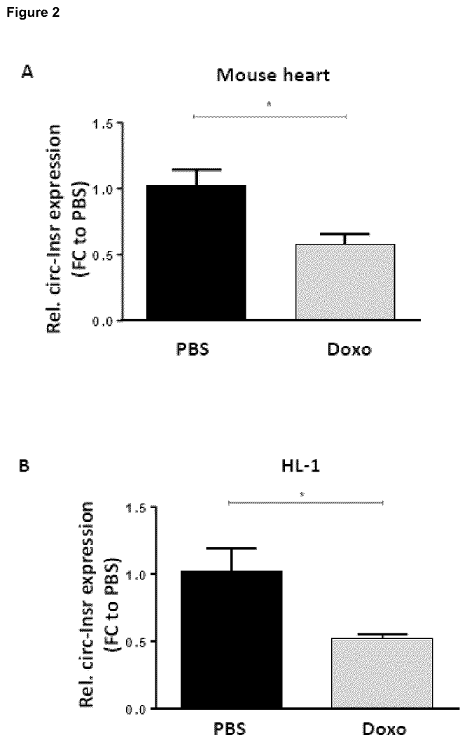

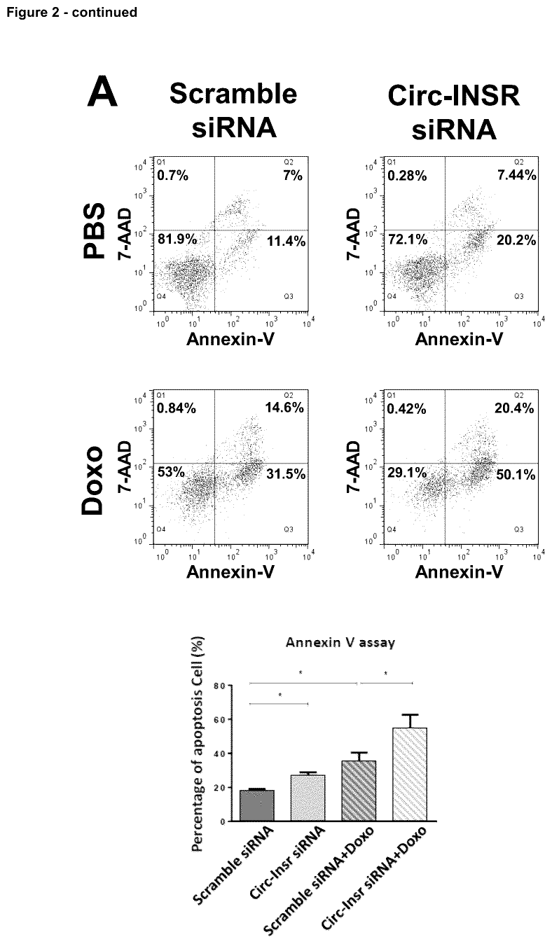

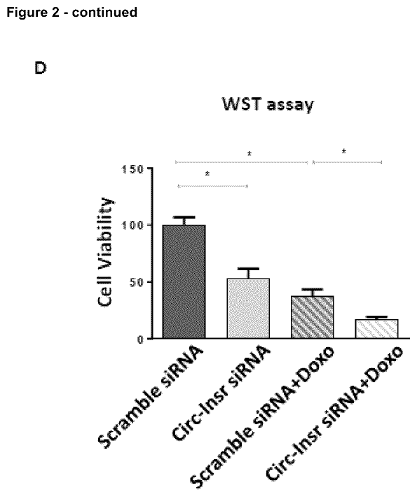

FIG. 2: Loss of circINSR augments doxorubicin-induced cardiomyocyte apoptosis. (A) Relative expression of circ-Insr in mouse heart treated with PBS control or doxorubicin (n=3, doxorubicin (5 mg/kg) treatment once a week for consecutive 5 weeks); (B) Relative expression of circ-Insr in doxorubicin treated HL-1 cells (n=3, Doxorubicin (0.25 μmol/L) treatment for 48 h); (C) Annexin-V (late apotosis) and 7-AAD (early apoptosis) staining of scramble siRNA and circ-Insr siRNA treated cells with/without doxorubicin (n≥3), FACS blots left and quantification of apoptotic cells right panel; (D) WST assay in circ-Insr siRNA HL-1 cells compared to scramble siRNA control (n≥3); (E) Annexin-V and 7-AAD staining of pcDNA3.1-Empty and pcDNA3.1-circ-Insr transfected cells with/without doxorubicin (n≥3); (F) WST assay (cell viability) in circ-Insr overexpression HL-1 cells compared to pcDNA3.1-Empty control (n≥3).

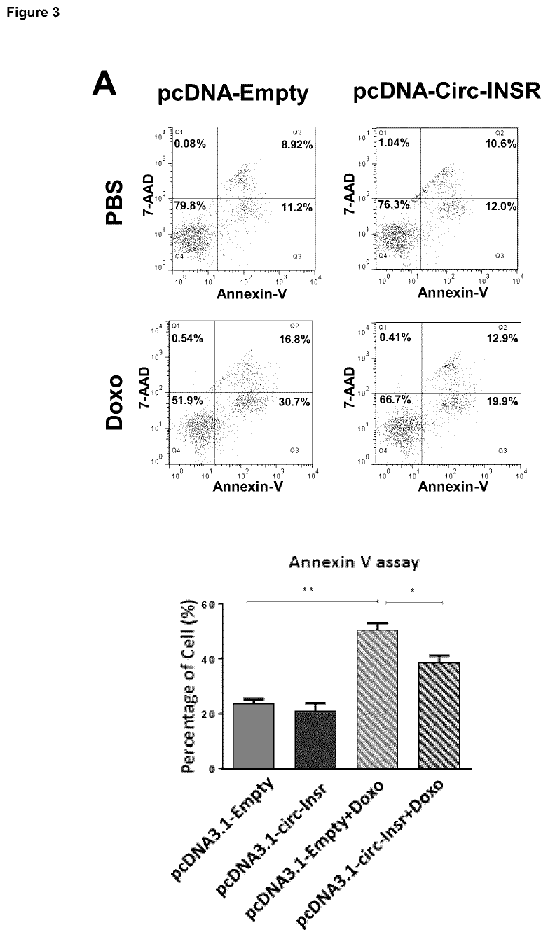

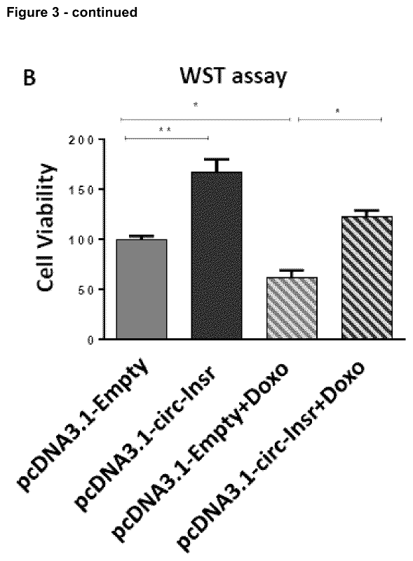

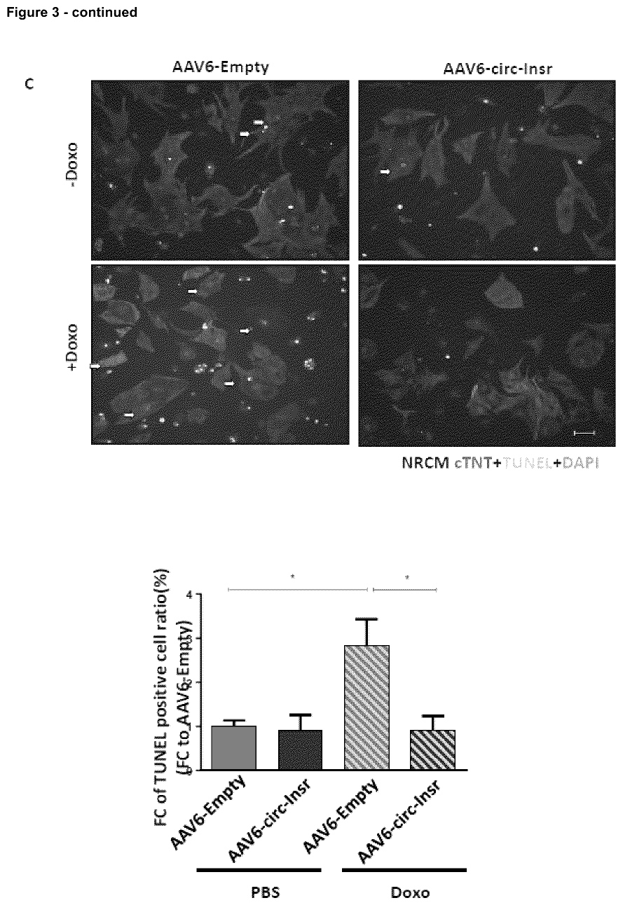

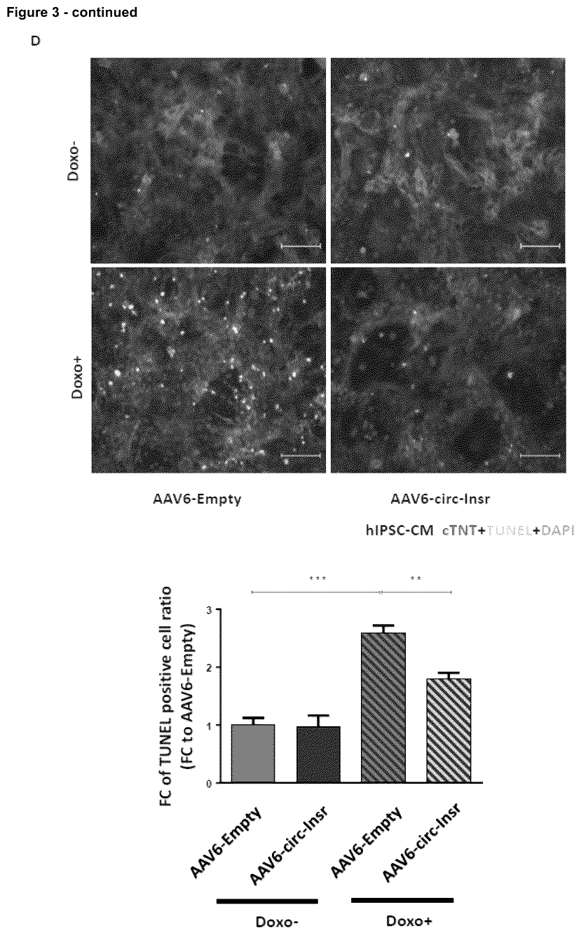

FIG. 3: Overexpression of circINSR rescues doxorubicin-induced toxicity in vitro. (A) Annexin-V (late apotosis) and 7-AAD (early apoptosis) staining of HL-1cells with/without doxorubicin transfected with circINSR overexpression construct or control vector (n≥3), FACS blots left and quantification of apoptotic cells right panel; (B) WST assay after doxorubicin treatment in circ-Insr overexpressing HL-1 cells compared to empty vector control (n≥3); (C) TUNEL (terminal deoxynucleotidyl transferase dUTP nick end labeling; green) staining of apoptotic nuclei in NRCMs (red-cTNT) transduced with either AAV6-Empty control or AAV6-circ-Insr, in presence or absence of doxorubicin (n=3, Scale bar, 100 μm). *P≤0.05; **P≤0.01; ***P≤0.001. FC=fold change; Rel.=Relative; (D) TUNEL (terminal deoxynucleotidyl transferase dUTP nick end labeling; green) staining of apoptotic nuclei in human iPSC-derived cardiomyocytes (red-cTNT) transduced with either AAV6-Empty control or AAV6-circ-Insr, in presence or absence of doxorubicin (n=3, Scale bar, 100 μm). *P≤0.05; **P≤0.01; ***P≤0.001.

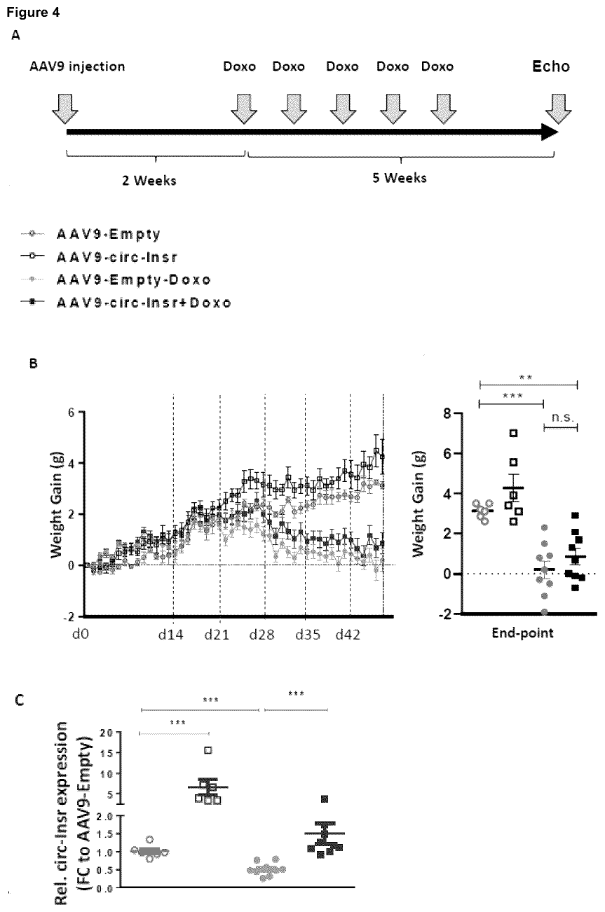

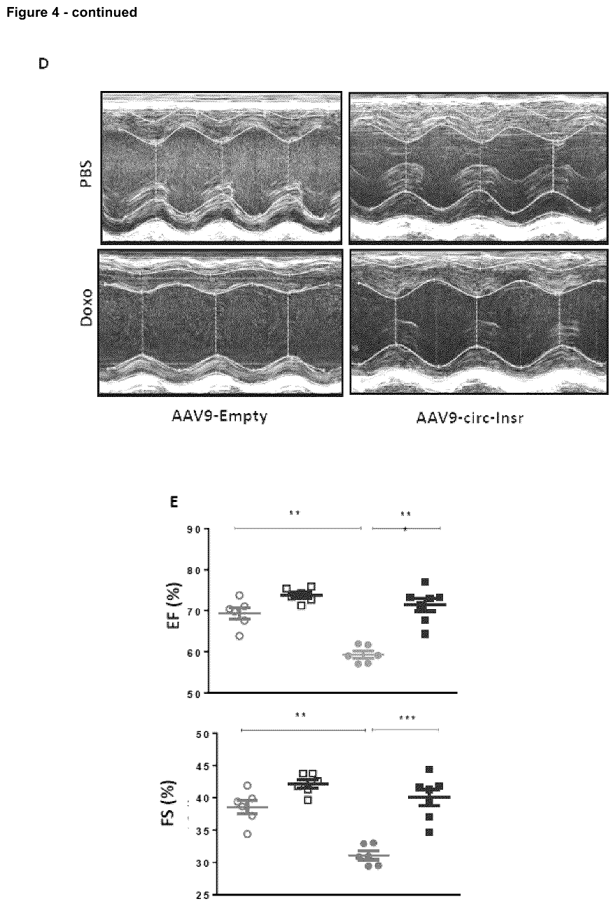

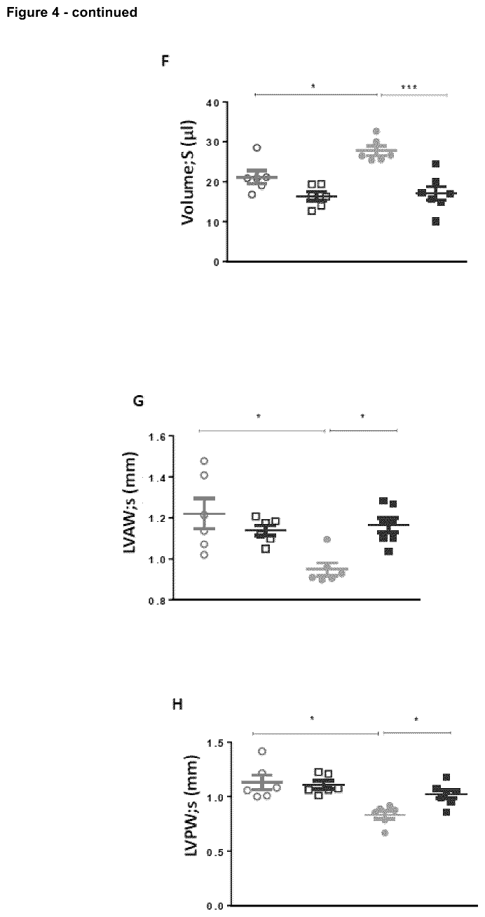

FIG. 4: Circ-Insr overexpression rescues doxorubicin-induced cardiotoxicity in vivo. (A) Schematic representation of the in vivo animal model and treatment scheme.; (B) Body weight changes during the experiment (n=6-9 each group); (C) Relative expression of linear-Insr and circ-Insr in AAV9-circ-Insr and AAV9-Empty heart tissue with or without doxorubicin treatment (n=6-9 each group); (D) Representative images of echocardiography analysis; (E) Ejection fraction and fractional shortening values; (F) Interventricular volume (systole); (G) Left-ventricular anterior wall thickness in systole; (H) Left-ventricular anterior wall thickness in systole. All parameters in AAV9-circ-Insr and AAV9-Empty treated mice with or without doxorubicin (n=6-7 each group).

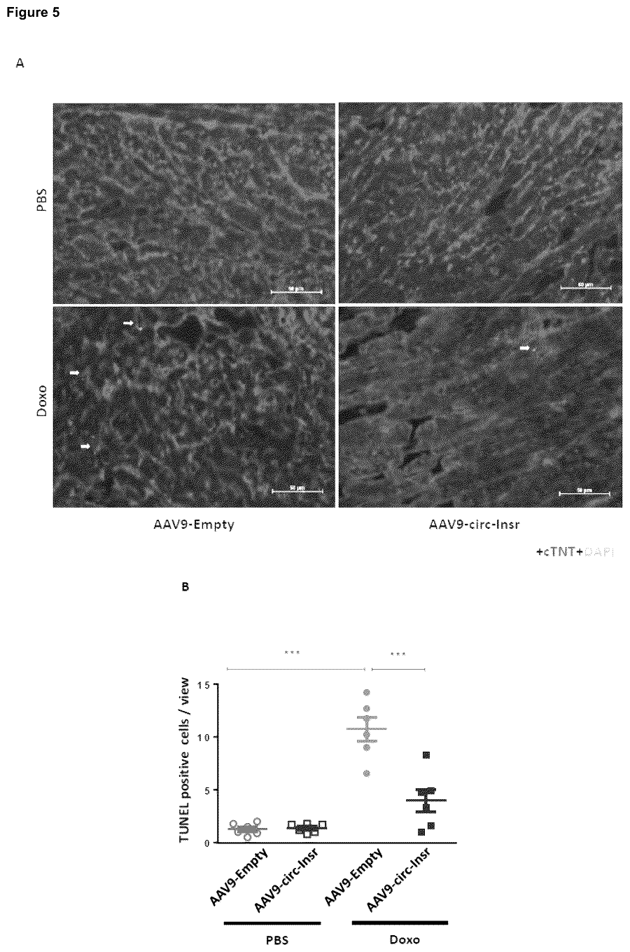

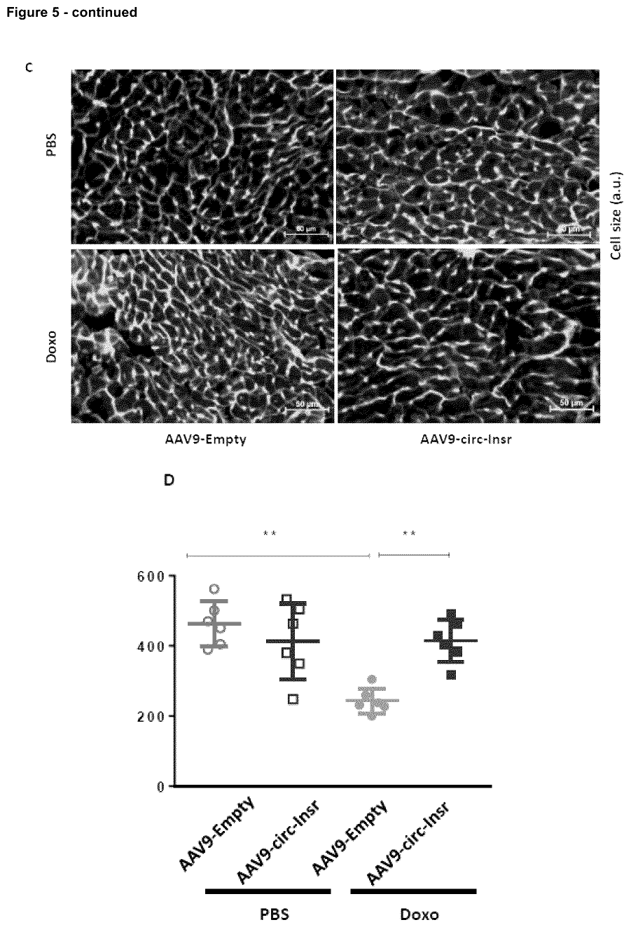

FIG. 5: (A) TUNEL staining in AAV9-Empty and AAV9-circ-Insr heart sections (n=6 each group, Scale bar, 50 μm), White arrows indicate TUNEL positive cells; (B) Quantification of TUNEL positive cardiomyocytes, *P<0.05; **P<0.01; ***P≤0.001; (B) Representative images of ardiomyocyte cell size measurement by Wheat germ agglutinin (WGA) staining of transversal heart tissue sections; (D) quantification of WGA staining (n=6 mice for each group, Scale bar, 50 μm).

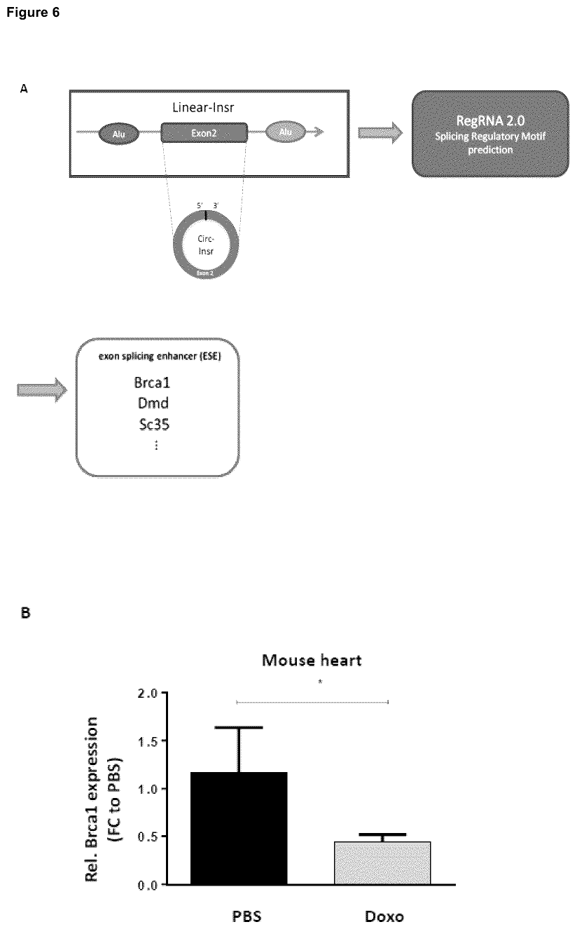

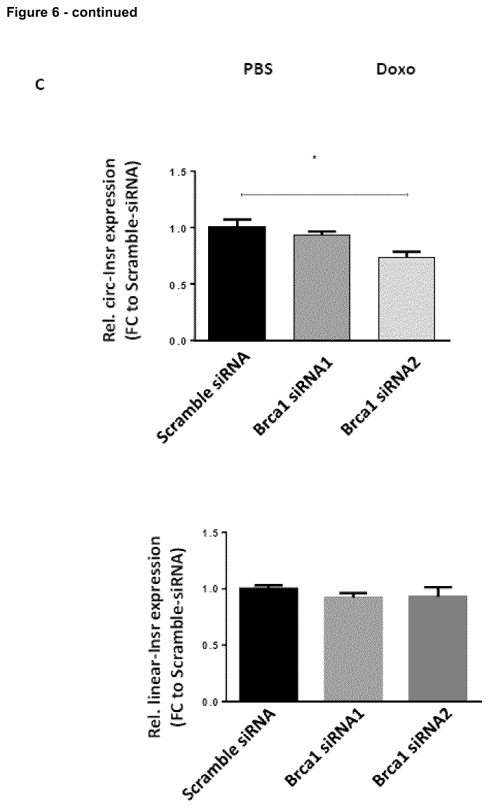

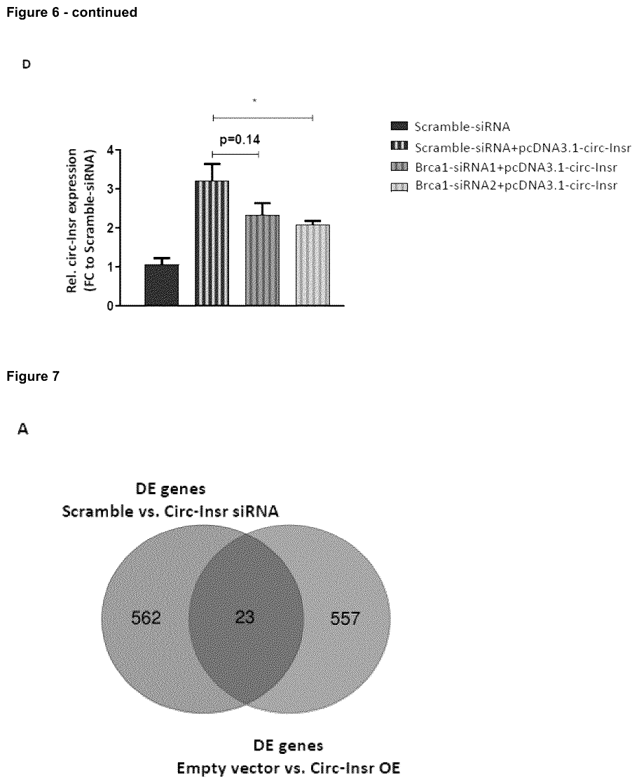

FIG. 6: Transcriptional Regulation of circ-Insr. (A) Prediction of the upstream regulator of circ-Insr using the software tool RegRNA2.0; (B) Relative expression of Brca1 in mouse heart treated with PBS control or doxorubicin (n=3-7) (C) Relative expression of circ-Insr (left) and linear Insr (right) in HL-1 cells treated with Brca1-siRNA (n=4); (D) Relative expression of circ-Insr in pcDNA3.1-circ-Insr transfected HL-1 cells co-treated with Brca1-siRNA (n=4),*P≤0.05.

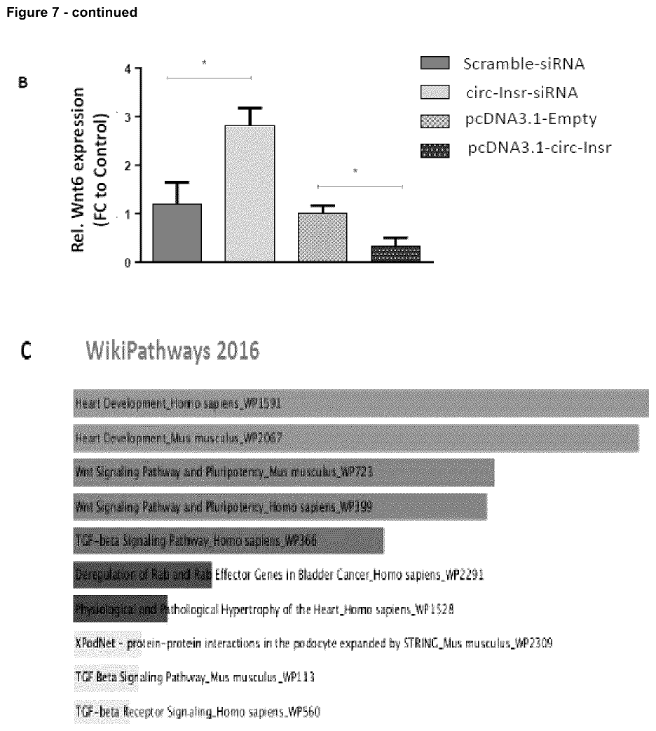

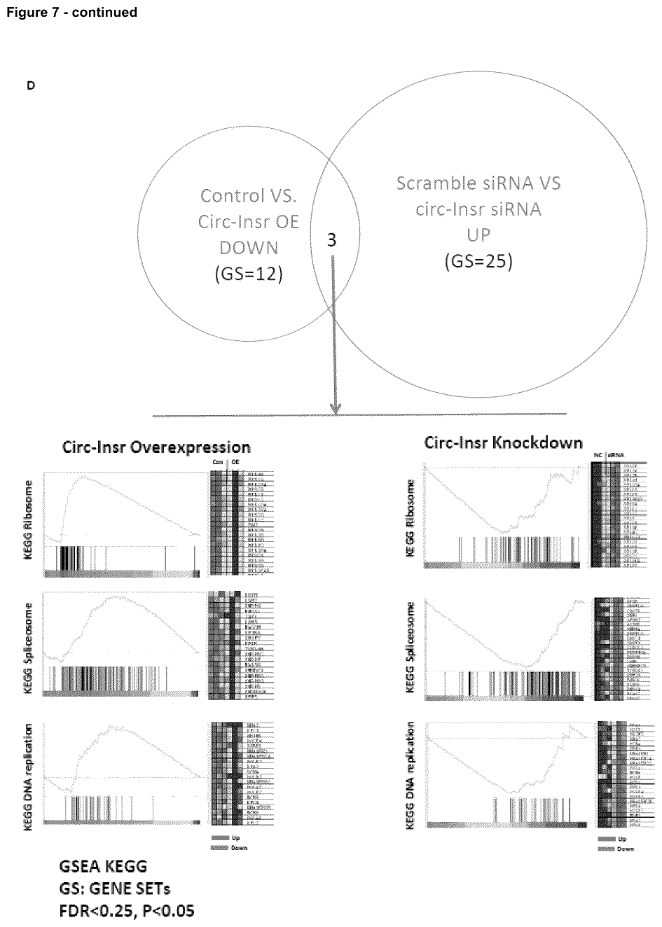

FIG. 7: Circ-Insr target genes and putative pathway analysis. (A) Venn diagram of dysregulated gene after RNA-seq in HL-1 after overexpression or knock-down of circINSR (B) Confirmation of RNAseq results. Relative expression of Wnt6 in HL-1 cells treated with circ-Insr-siRNA or pcDNA3.1-circ-Insr compare to control (n=3); (C) Functional enrichment of deregulated gene in WikiPathways 2016 data base; (D) GSEA analysis of RNA-seq in HL-1 after overexpression or knock-down of circINSR, three reciprocally regulated gene sets are depicted.

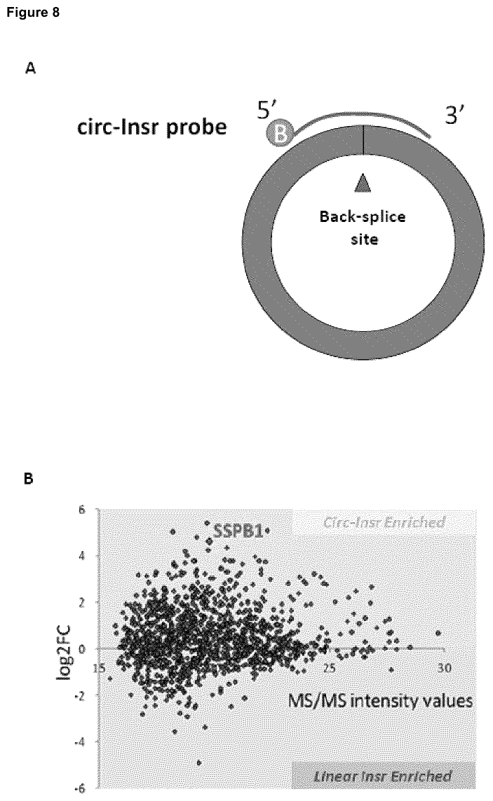

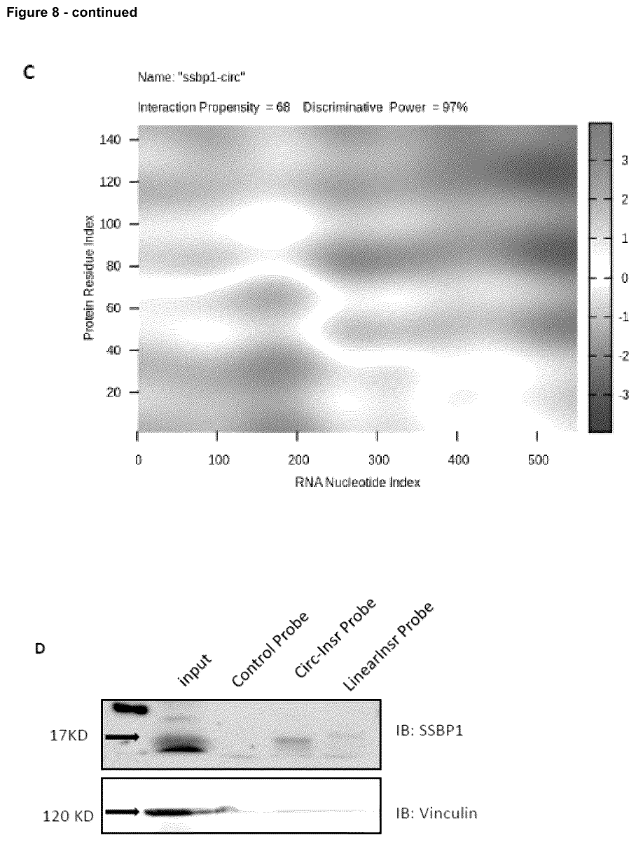

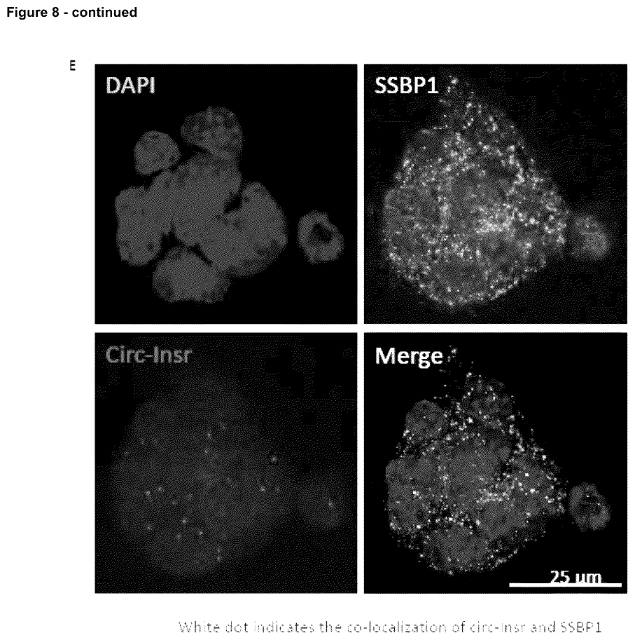

FIG. 8: CircINSR interacts physically with SSBP1. (A) Schematic representation of the RNA pull-down approach using a biotin-labelled DNA Probe specific for the circINSR backsplice site; (B) Dot plot graph showing the relative protein enrichment and MS/MS intensity values between circINSR probe and a control probe targeting the linear INSR mRNA; (C) Prediction of RNA-protein interaction potential of circ-Insr with SSBP1 using the catRAPID algorithm; (D) Western blot of SSBP1 after circ-Insr and linear-Insr RNA pulldown in 3T3 cells (n=2); (E) Immuno-RNA-FISH staining of HL-1 cells with SSBP1 specific antibodies and a circINSR specific FISH probe. White dots in the merged panel indicate co-localisation.

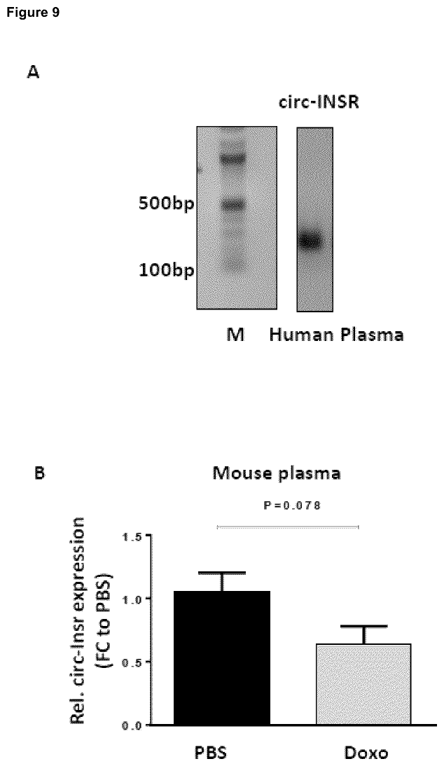

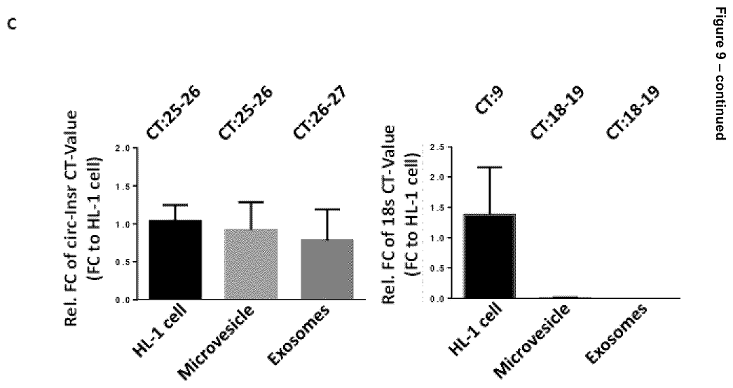

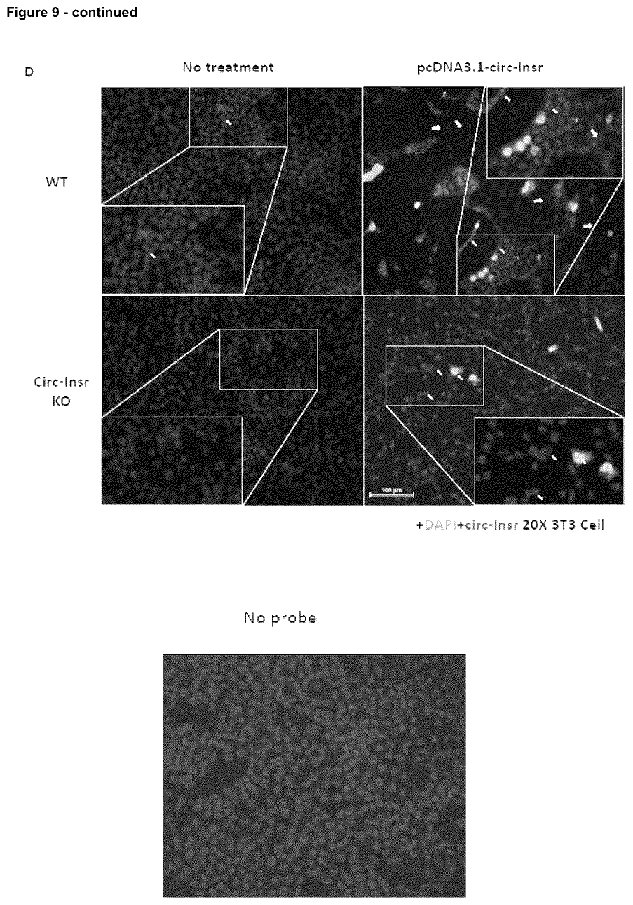

FIG. 9: Circ-Insr is a secreted RNA detectable in plasma. (A) Agarose gel electrophoresis of circ-INSR PCR products in human plasma; (B) Relative expression of circ-Insr in mouse plasma with PBS control or doxorubicin (n=6-7); (C) Circ-Insr and 18s levels (negative control) in HL-1 cells, micro vesicles and exosomes (n=3-5); (D) In situ hybridization of circ-Insr in WT and circ-Insr-KO 3T3 cells transfected with pcDNA3.1-circ-Insr (Scale bar, 100 μm); Note, circ-Insr receptor can be detected in GFP negative cells, suggesting a cell-to-cell transfer.

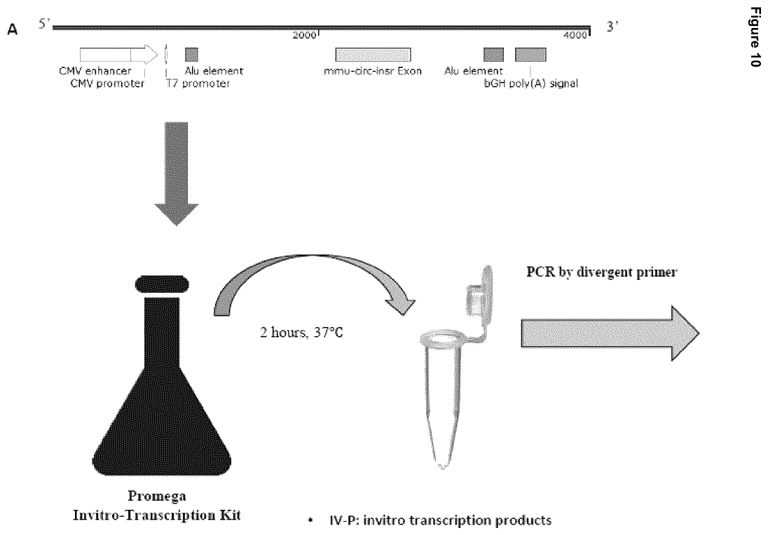

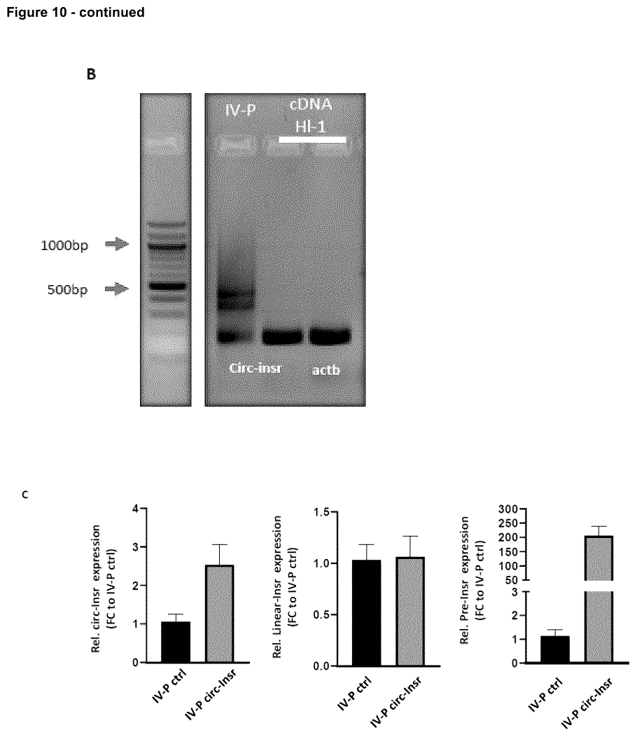

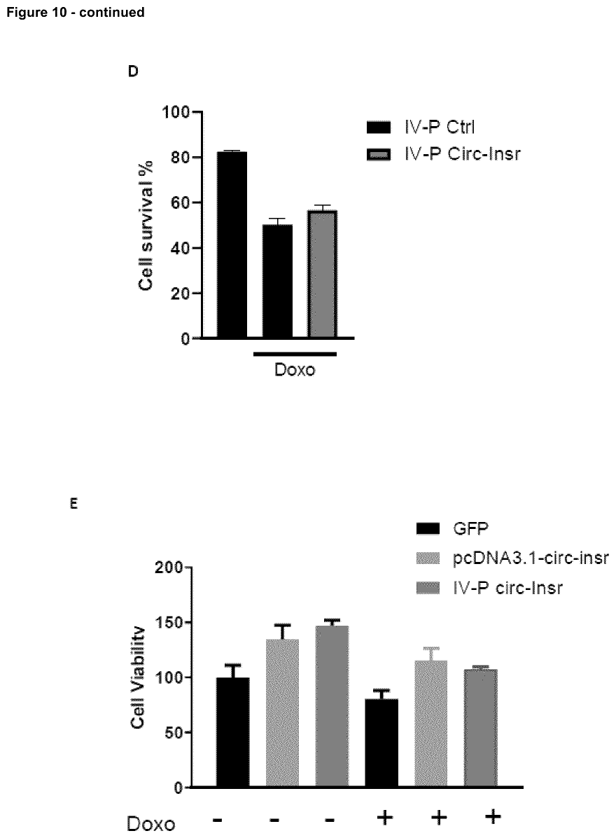

FIG. 10: In vitro transcription Circ-Insr rescues doxorubicin-induced HL-1 apoptosis. (A) Schematic drawing of in vitro transcription circ-Insr production procedures. (B) Gel electrophoresis results in circ-Insr in vitro transcription circ-Insr products (IV-P) and HL-1 cells. (C) Relative expression of circ-Insr, linear-Insr and Pre-Insr in HL-1 cells transfected with in vitro transcription circ-Insr. (D) Annexin-V (early apoptosis) and 7-AAD (late apoptosis) staining of control and circ-Insr in vitro transcription products transfected cells with/without doxorubicin treatment. Representative images left; quantification right (n=3, technical repeats). (E) WST assay (cell viability) in circ-Insr in vitro transcription products transfected cardiomyocyte-like HL-1 cells compared to control (n=3, technical repeats); FC=fold change.

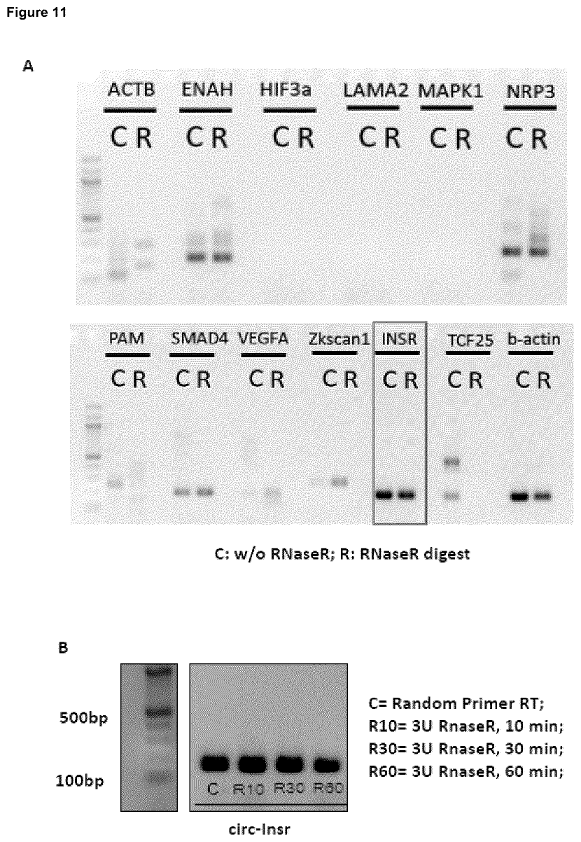

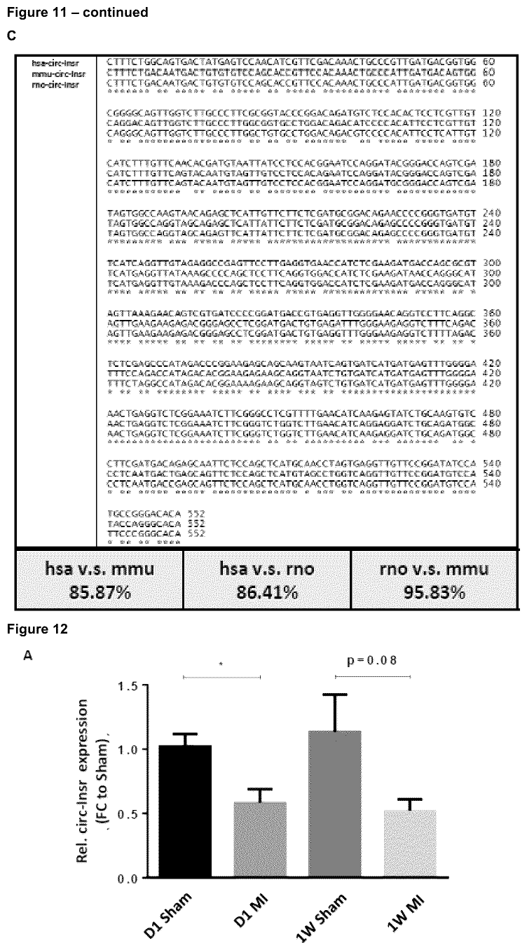

FIG. 11: Circ-Insr conservation in mouse, rat and human. (A) PCR results of selected candidates with or without RNase R treatment; (B) Pairwise alignment of the human and mouse circ-Insr sequence; (B) RNase R treatment for different incubation time indicating high stability of circINSR; (C) Multiple sequence alignment of circINSR from human, mouse and rat, sequence identity in pairwise comparison is indicated.

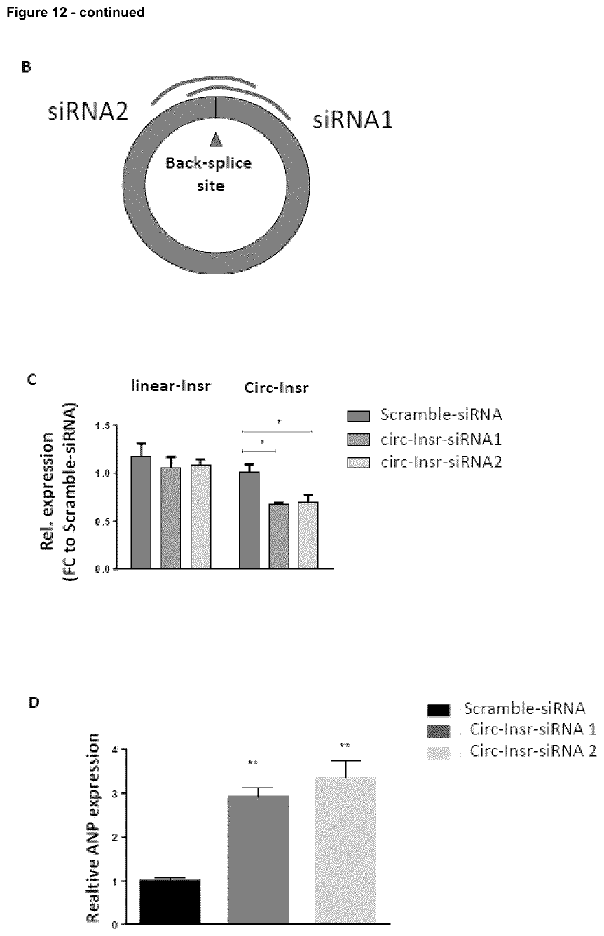

FIG. 12: (A) Comparison of circINSR expression in hearts from mice 1 day and 1 week after myocardial infarction compared to Sham control; (B) Schematic drawing of circ-Insr (red circle) and relative position of back-splice site specific siRNAs loci (blue lines); (C) Relative expression of linear-Insr and circ-Insr in HL-1 cells treated with circ-Insr siRNA compare to scramble siRNA (n≥3); (D) Relative mRNA expression of the cardiac stress marker ANP in HL-1 cells treated with circ-Insr siRNA compare to scramble siRNA (n≥3).

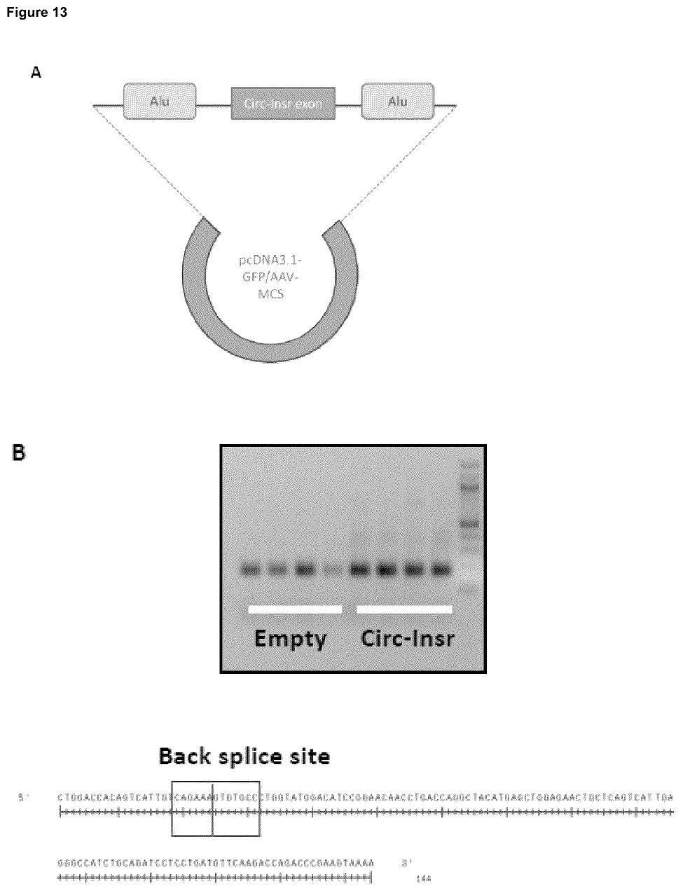

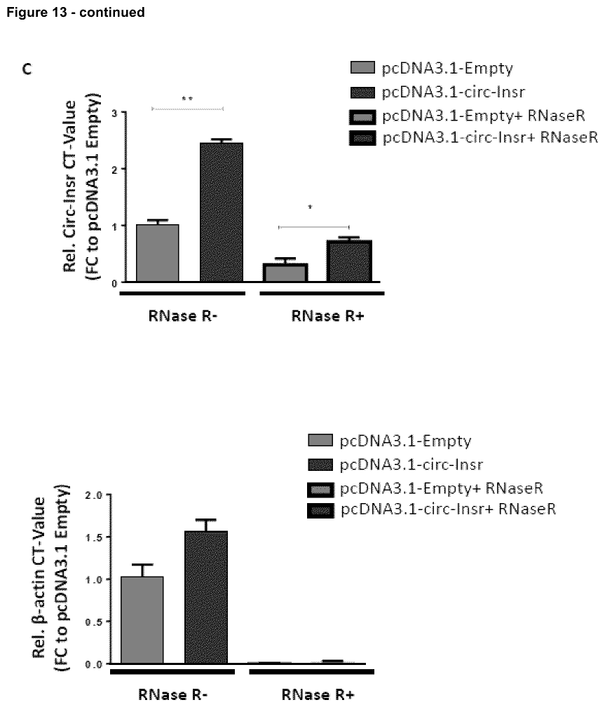

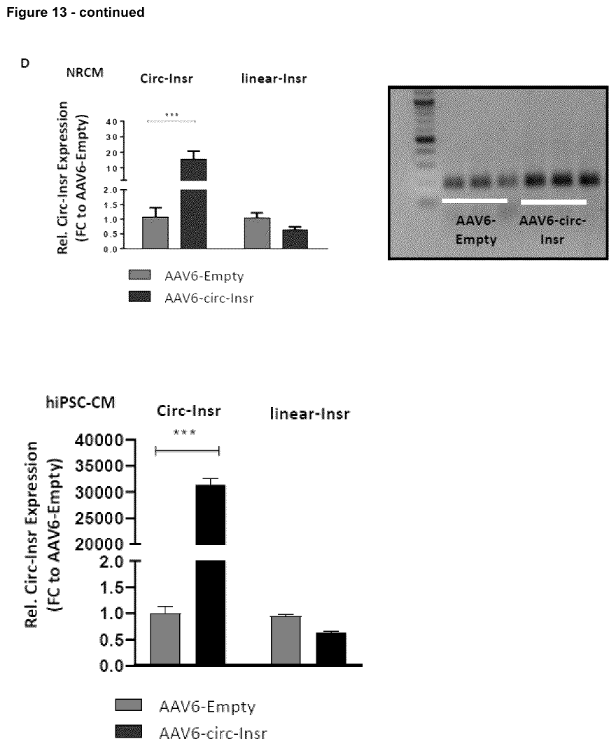

FIG. 13: (A) Schematic representation of the circINSR overexpression vector; (B) Gel electrophoresis and Sanger sequencing result of the circ-Insr PCR products in circ-Insr overexpression HL-1 cells; (C) Relative expression of circ-Insr and β-actin in pcDNA3.1-circ-Insr transfected cells (n=4, RNA treated with/with RNase R); (D) Relative expression of linear-Insr and circ-Insr in NRCMs and human iPSC-derived cardiomyocytes treated with AAV6-circ-Insr compared to AAV6-Empty (n=3) and agarose gel electrophoresis image of circ-Insr PCR products in AAV6-circ-Insr infected NRCMs.

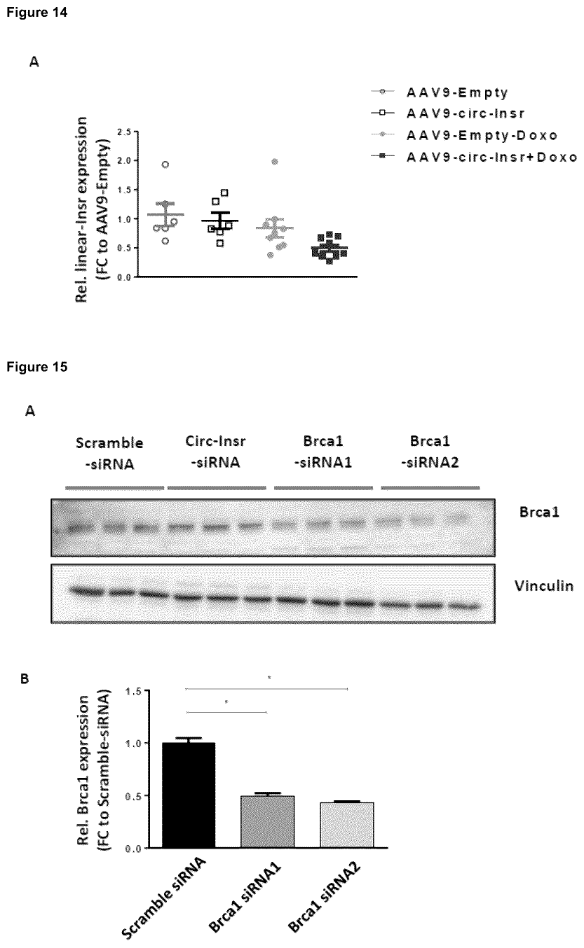

FIG. 14: (A) Relative INSR mRNA expression in AAV9-circ-Insr and AAV9-Empty treated mice with or without doxorubicin (n=6-7 each group).

FIG. 15: (A) Western blot of Brca1 in HL-1 cells treated with Brca1-siRNA and circ-Insr-siRNA (n=3); (B) Relative mRNA expression of Brca1 in HL-1 cells treated with Brca1-siRNA (n=4).

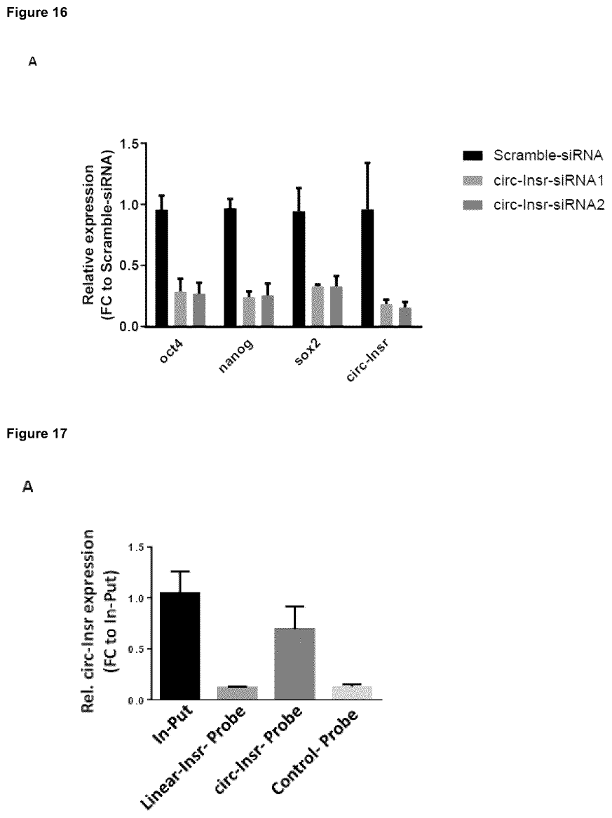

FIG. 16: Relative expression of the pluripotency genes Oct4, Nanog and Sox2 in human iPSC after treatment with circINSR siRNA.

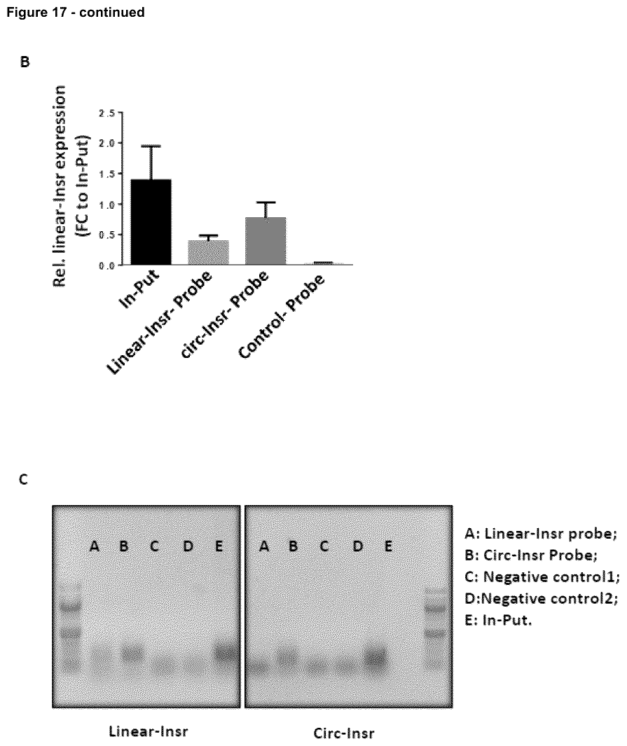

FIG. 17: (A) Relative expression/enrichment of circINSR before (input) and after RNA-pulldown with the indicated probes; (B) Relative expression/enrichment of linear INSR before (input) and after RNA-pulldown with the indicated probes; (C) Agarose gel electrophoresis of circ-Insr and linear-Insr PCR products in 3T3 cells after circ-Insr and linear-Insr RNA pull-down.

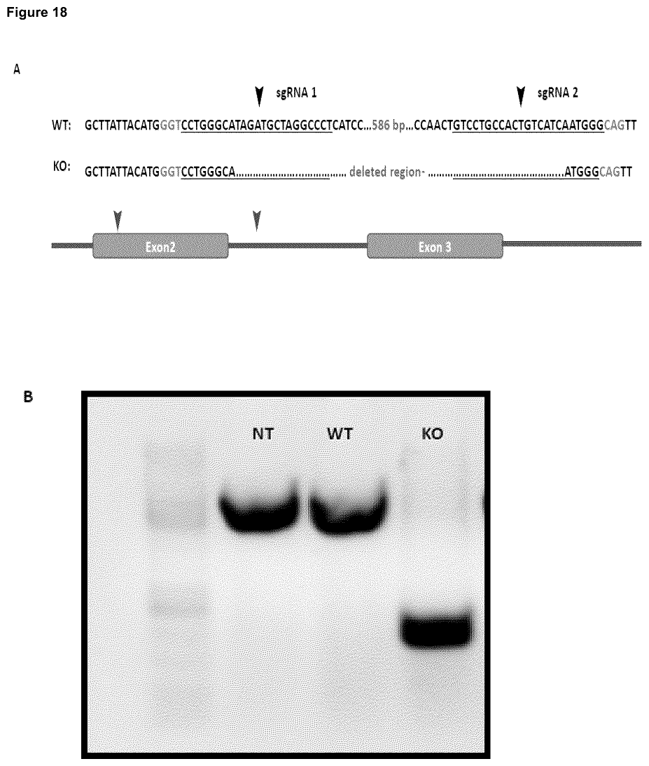

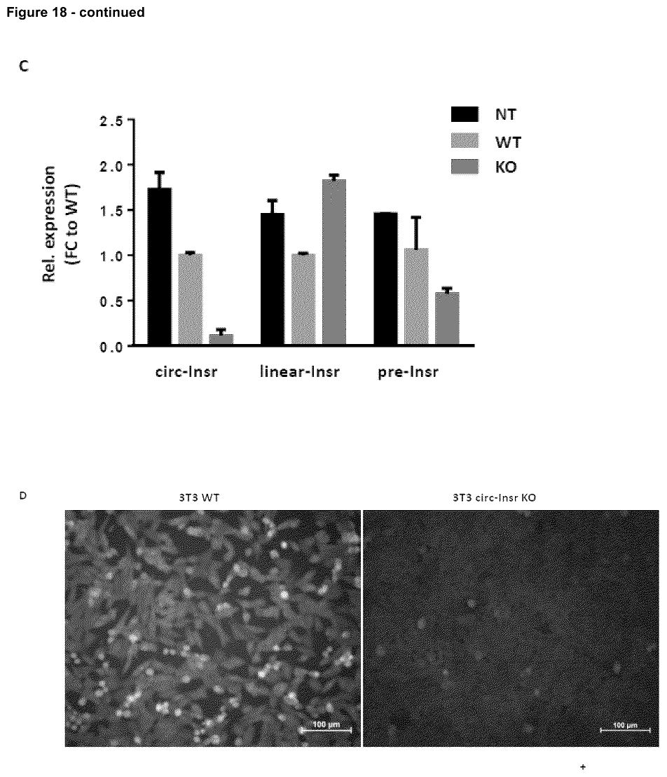

FIG. 18: (A) Schematic representation of CRISPR/Cas9-mediated knock-out of circ-Insr in 3T3 cells; (B) Agarose gel electrophoresis results indicating the successful knock-out of circINSR after sgRNA transfection. NT=non-transfected control, WT=wildtype control; KO=knock-out; (3) Relative expression of circ-Insr, linear-Insr and pre-Insr in WT, NT and KO 3T3 cells; (D) Immunofluorescent staining of Insr in WT and circ-Insr-KO 3T3 cells (Scale bar, 100 μm).

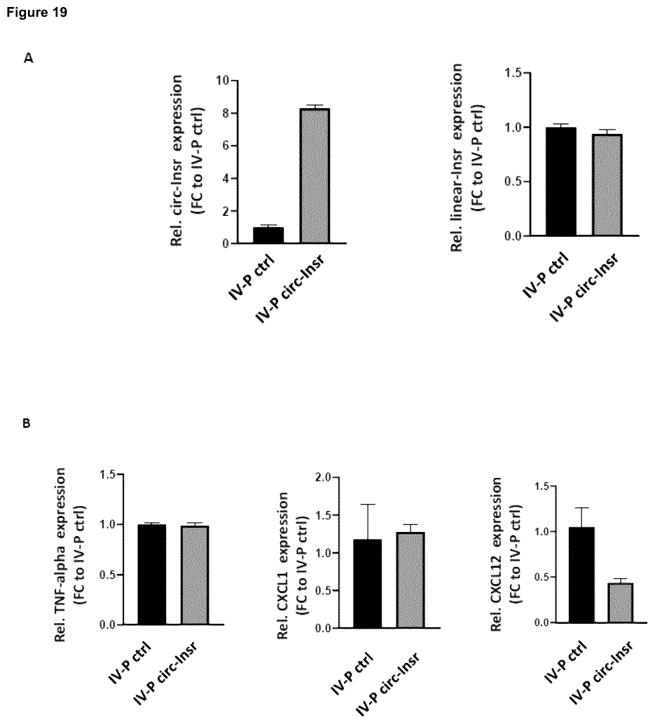

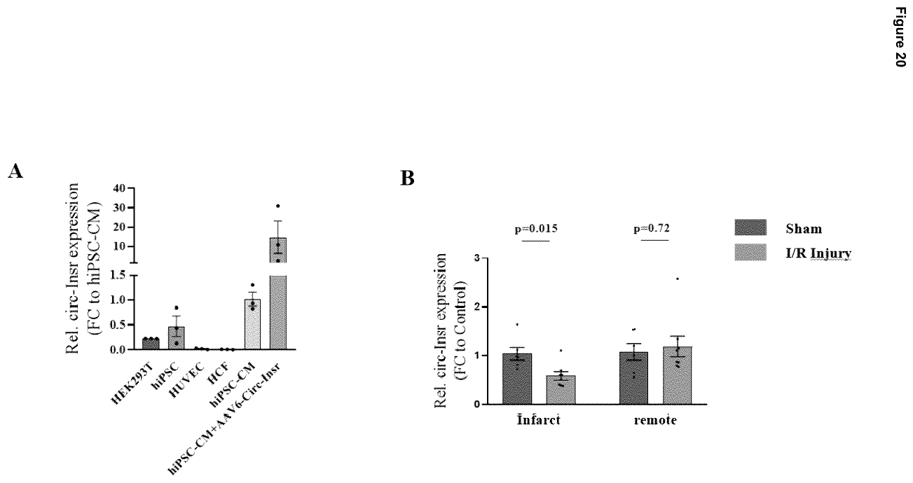

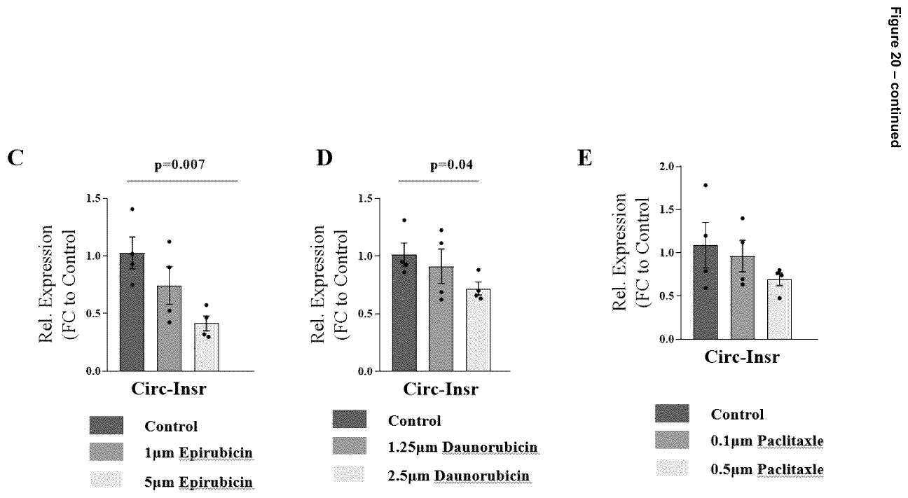

FIG. 19: (A) Relative expression of circ-Insr and linear-Insr in RAW264.7 cells transfected with in vitro transcription circ-Insr. (B) Relative expression of TNF-alpha, CXCL1 and CXCL12 in RAW264.7 cells transfected with in vitro transcription circ-Insr.

FIG. 20: Expression of Circ-Insr in human and mouse samples. (A) Relative expression of circ-Insr in HEK293T, hiPSCs (Human Induced Pluripotent Stem Cells), HUVECs (Human Umbilical Vein Endothelial Cells), HCFs (Human Cardiac Fibroblasts), hiPSC-CMs (Human Induced Pluripotent Stem Cells derived cardiomyocytes) (N=3, independent experiments); (B) Relative expression of circ-Insr in infarct and remote region from I/R injury heart compared to Sham control (n=6 to 9 mice per group); (C) Relative expression of circ-Insr in HL1 cells treated with Epirubicin (1 μM and 5 μM, 24 h, n=4); (D) Relative expression of circ-Insr in HL1 cells treated with Daunorubicin (1.25 UM and 2.5 μM, 24 h, n=4); (E) Relative expression of circ-Insr in HL1 cells treated with Paclitaxle (0.1 μM and 0.5 μM, 24 h, n=4).

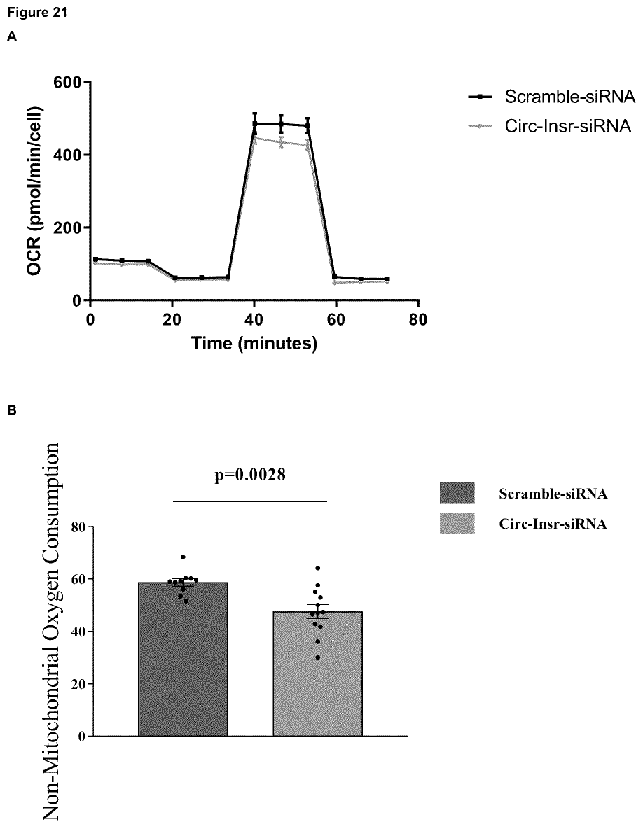

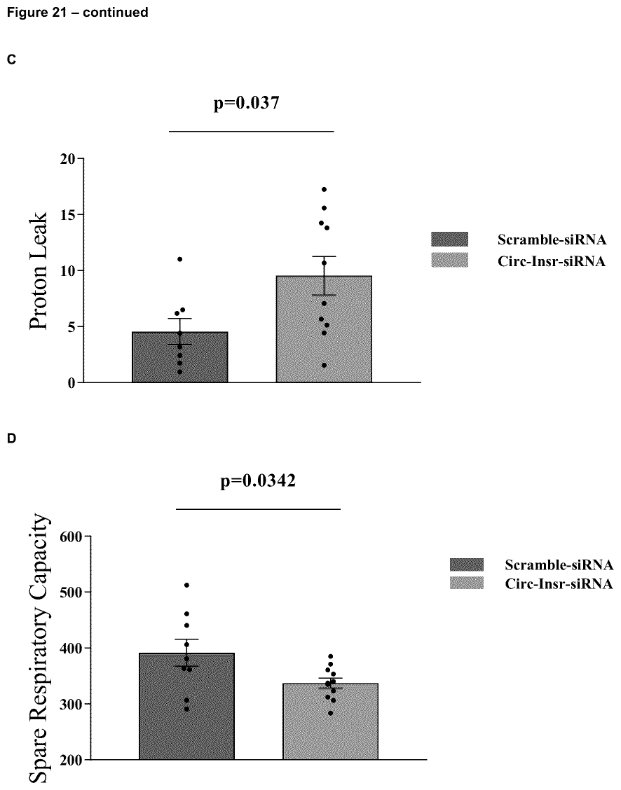

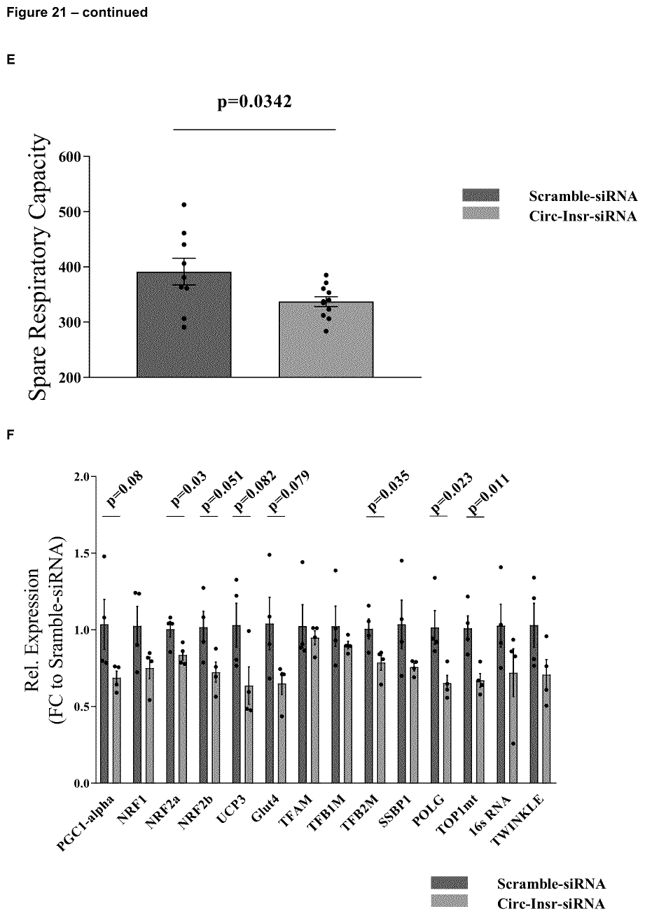

FIG. 21: Loss of Circ-Insr impaired metabolic activity in cardiomyocytes. (A) Mitochondrial respiration reflected by OCR levels was detected in NMCM (neonatal Mouse Cardiomyocytes) treated with circ-Insr siRNA compare to scramble siRNA under basal conditions or following the addition of oligomycin (1 μM), the uncoupler FCCP (1 μM) or the electron transport inhibitor Rotenone (0.5 UM) (n=9 to 10 per group); (B) Non-Mitochondrial Oxygen Consumption of NMCM treated with circ-Insr siRNA compare to scramble siRNA; (C) Proton Leak of NMCM treated with circ-Insr siRNA compare to scramble siRNA; (D) Spare Respiratory Capacity of NMCM treated with circ-Insr siRNA compare to scramble siRNA; (E) Coupling Efficiency of NMCM treated with circ-Insr siRNA compare to scramble siRNA; (F) Relative expression of mitochondrial biogenesis-related genes in NRCM (Neonatal Rat Cardiomyocytes) treated with circ-Insr siRNA compare to scramble siRNA (n=4).

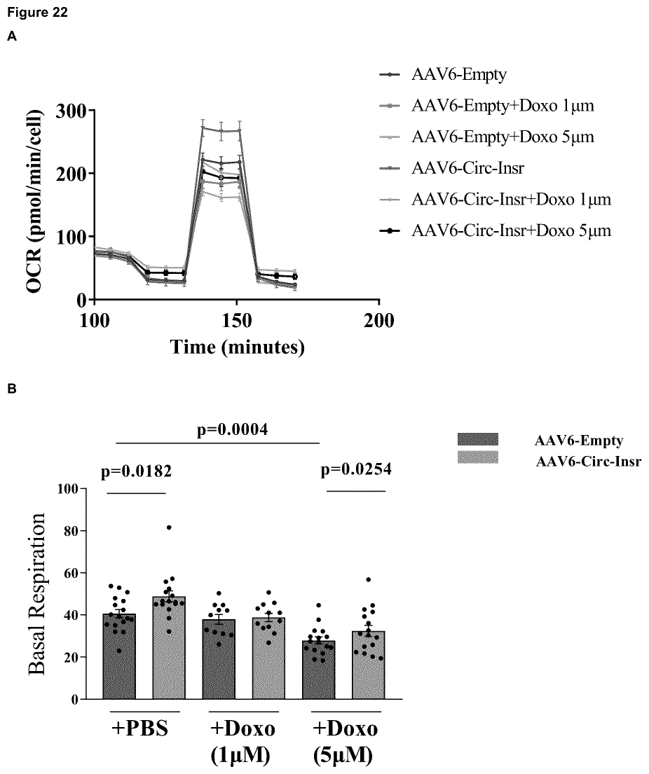

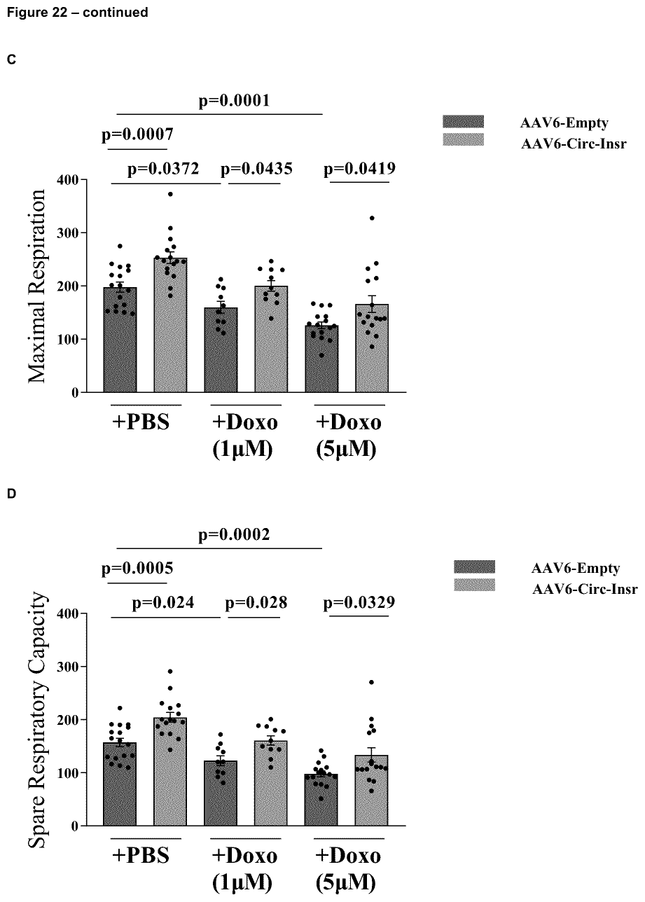

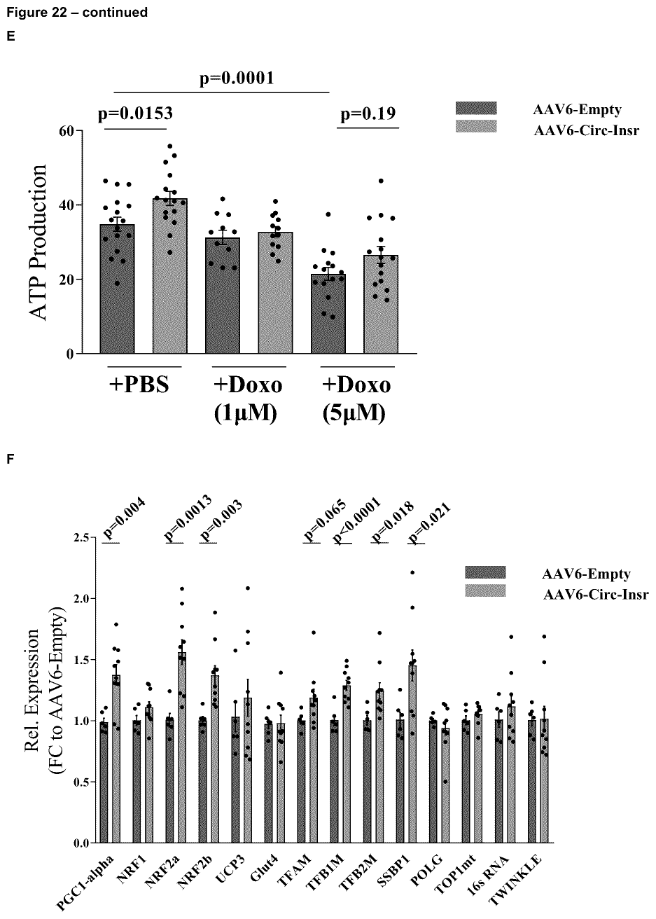

FIG. 22: Circ-Insr rescues doxorubicin-induced cardiomyocytes metabolic disorder. (A) Mitochondrial respiration reflected by OCR levels was detected in NMCM treated with AAV6-circ-Insr compare to AAV6-Empty under basal conditions or doxorubicin (1 μM and 5 μM, 2 h), following with the assay treatment of oligomycin (1 μM), the uncoupler FCCP (1 μM) or the electron transport inhibitor Rotenone (0.5 μM) (n=11 to 17 per group); (B) Basal Respiration of NMCM treated with AAV6-circ-Insr compare to AAV6-Empty under basal conditions or doxorubicin (1 UM and 5 μM, 2 h); (C) Maximal Respiration of NMCM treated with AAV6-circ-Insr compare to AAV6-Empty under basal conditions or doxorubicin (1 μM and 5 μM, 2 h); (D) Spare Respiratory Capacity of NMCM treated with AAV6-circ-Insr compare to AAV6-Empty under basal conditions or doxorubicin (1 μM and 5 μM, 2 h); (E) ATP Production of NMCM treated with AAV6-circ-Insr compare to AAV6-Empty under basal conditions or doxorubicin (1 μM and 5 μM, 2 h); (F) Relative expression of mitochondrial biogenesis-related genes in NRCM AAV6-circ-Insr compare to AAV6-Empty under doxorubicin treatment (0.5 μM 48 h) (n=6 vs. 9).

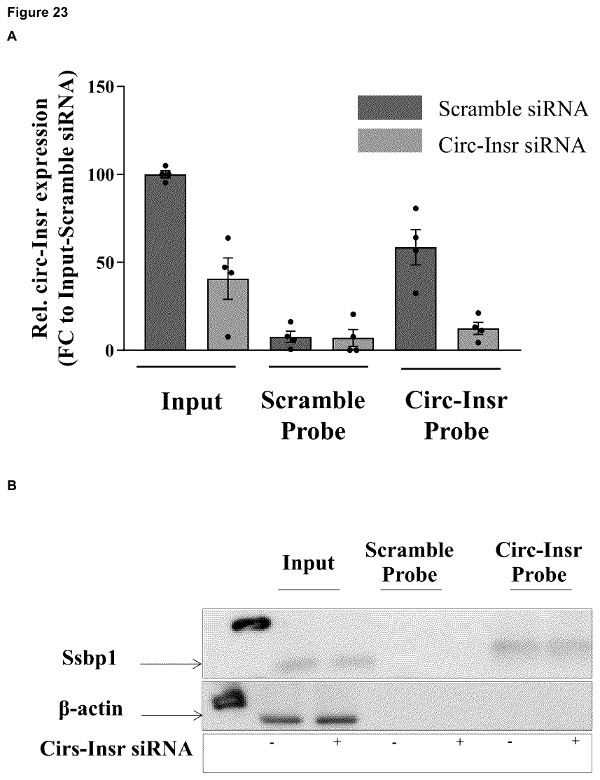

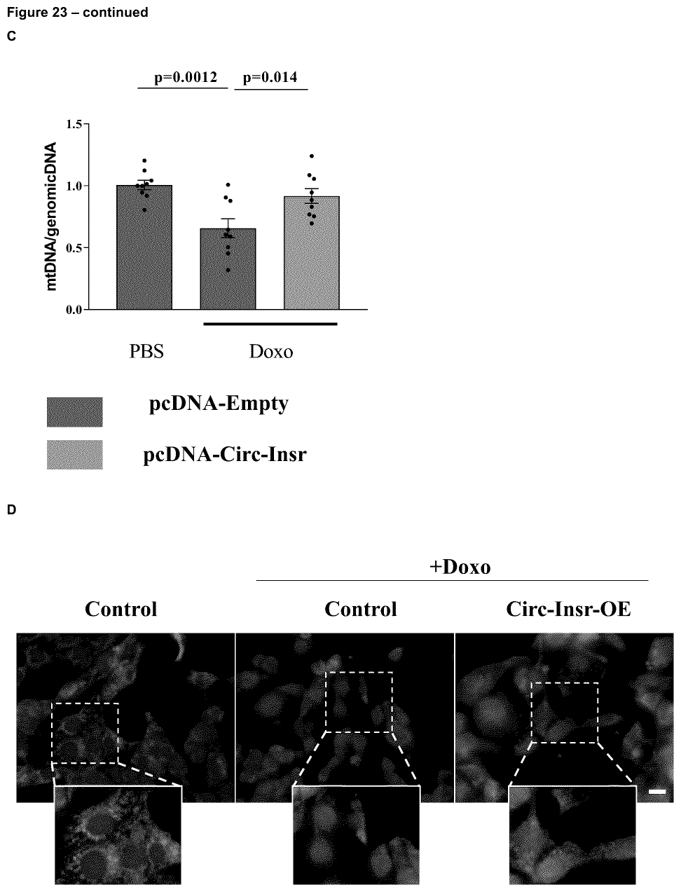

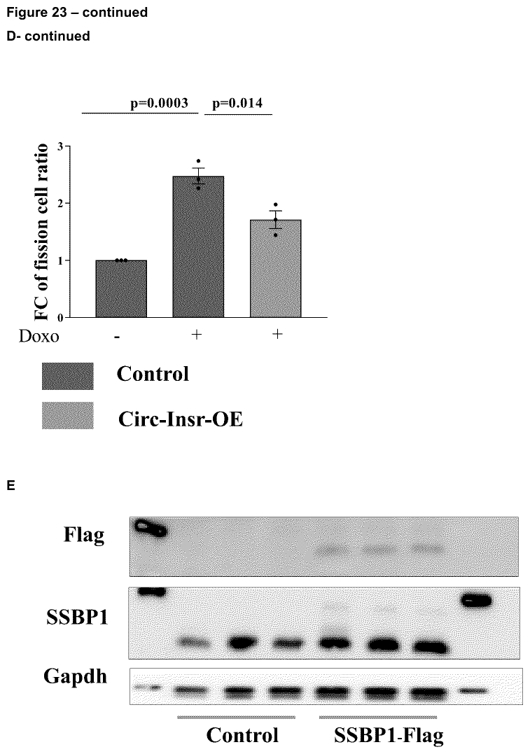

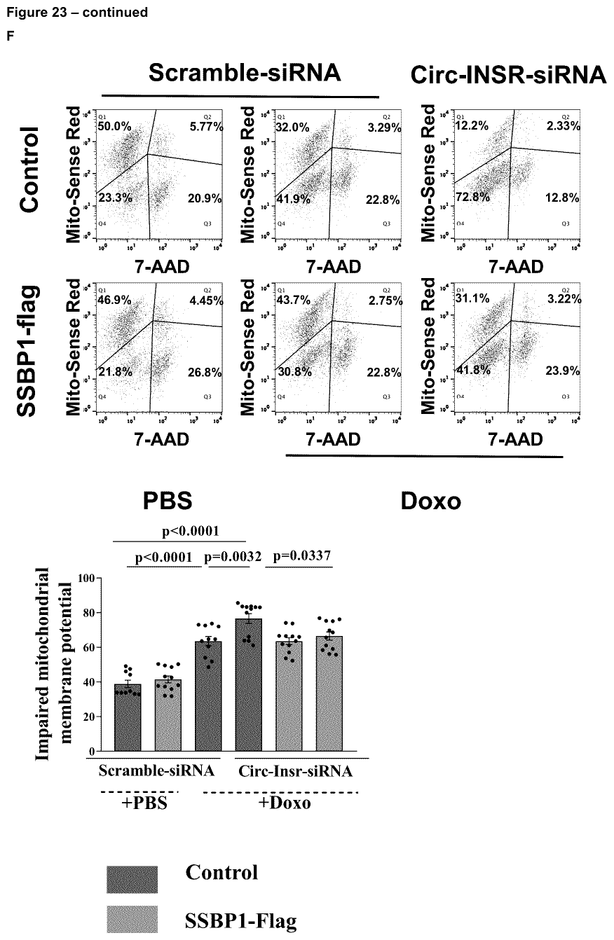

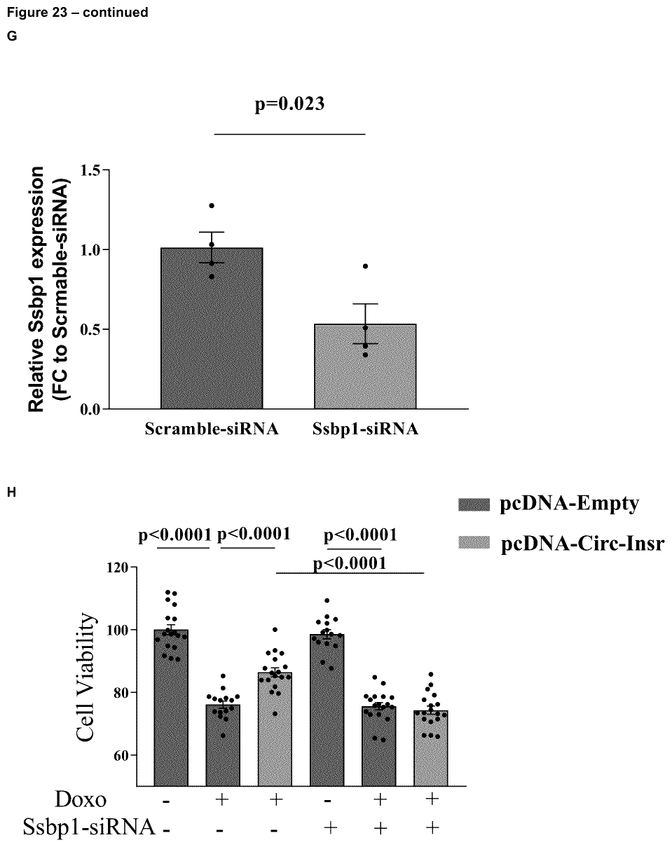

FIG. 23: Circ-Insr regulates mitochondrial function through SSBP1 in cardiomyocytes. (A) Relative expression of circ-Insr in HL-1 cells treated with circ-Insr siRNA following with circ-Insr pulldown assay compare to control; (B) Relative protein level of SSBP1 in HL-1 cells treated with circ-Insr siRNA following with circ-Insr pulldown assay compare to control; (C) qRT-PCR analysis mtDNA copy number in HL-1 cells treated with circ-Insr overexpression plasmid and doxorubicin (0.25 μM 48 h) compare to control (n=8); (D) Mito-tracker staining assay in HL-1 cells treated with circ-Insr overexpression plasmid and doxorubicin (0.25 μM 48 h) compare to control (n=3); (E) Relative protein level of SSBP1 and Flag in HL-1 cells treated with SSBP1-flag overexpression plasmid compare to control plasmid (n=3); (F) Mitochondrial membrane potential assay in HL-1 cells treated with circ-Insr-siRNA, SSBP1-flag overexpression plasmid and doxorubicin (0.25 μM 48 h) (n=10 to 12 per group); (G) Relative SSBP1 expression in HL-1 cells treated with SSBP1-siRNA compare to scramble siRNA (n=3); (H) WST assay in HL-1 cells treated with circ-Insr overexpression plasmids, SSBP1-siRNA plasmid and doxorubicin (0.25 UM 48 h).

The Examples illustrate the invention.

### EXAMPLE 1—IDENTIFICATION AND VALIDATION OF CONSERVED CIRCRNA, CIRCINSR

First it was set out to generate cardiac circRNA expression profiles in tissue samples from human healthy and failing hearts, from mouse hearts with and without left ventricular hypertrophy induced by transverse aortic constriction (TAC) and from HL-1 cells, a murine cardiomyocyte-like cell line. Exploiting the circRNA unique backsplicing “sequence tag”, bioinformatics analyses was used to identify hundreds of annotated but also novel species-conserved circRNAs in these samples. Through stringent selection criteria (species conservation, cardiac function prediction of the host-gene and bioinformatics validation of backsplicing) ˜30 cardiac relevant circRNAs were identified (FIG. 1A). After wet lab validation by PCR amplification of the circle using divergent primers and test for RNaseR resistance (FIG. 11A,B), a circRNA formed through backsplicing of the second exon of the murine (mmu-circ-0014773), the human (hsa-circ-0000885) and the rat (not yet annotated) insulin receptor gene transcript (hereafter termed circINSR) emerged as lead circRNA candidate for further investigation (FIG. 1B). circINSR shows a high degree of sequence conservation (FIG. 11C) and backsplicing was confirmed by sanger sequencing in the 3 different species (FIG. 1C). Subcellular fractionation of HL1-cells revealed a predominantly cytoplasmic localization of circINSR which was further confirmed by qualitative RNA-FISH (FIG. 1D,E).

### EXAMPLE 2—RESCUE OF DOXORUBICIN-INDUCED CARDIOTOXICITY BY CIRCINSR IS SPECIES CONSERVED

In addition to the initial identification of dysregulated circINSR in human and murine heart failure samples, a significant decrease of cirINSR was identified in mouse hearts exposed to cardiotoxicity inducing agent doxorubicin and after myocardial infarction (FIG. 2A & FIG. 12A). Doxorubicin-mediated downregulation of circINSR was confirmed in HL-1 cells (FIG. 2B). For functional investigation of circINSR siRNAs were designed which specifically target the unique sequence stretch generated by back-splicing. Indeed, two siRNAs were found that degrade circINSR but not the linear transcript which led to an induction of the cardiac stress marker gene ANP (FIG. 12B-D). Since doxorubicin is known to induce cardiomyocyte apoptosis¹². Annexin V assays were performed in HL-1 cells treated with doxorubicin and simultaneously with siRNA against circINSR or scramble siRNA. Expectedly, doxorubicin treatment increased the number of apoptotic cells which was significantly augmented upon circINSR siRNA treatment. Interestingly, already under basal condition circINSR inhibition let to an increase of apoptotic cells (FIG. 2C). In line with this, the cell viability as determined in WST-1 assays was significantly diminished after siRNA or doxorubicin treatment and additive effects were observed upon simultaneous treatment (FIG. 2D).

In order to test whether counteracting endogenous downregulation of circINSR has beneficial effects on doxorubicin-induced cardiotoxicity exon 2 plus the ALU element containing up- and downstream intronic sequences (pairing of ALU elements facilitates circRNA formation¹³) was cloned into a pcDNA3.1 lentiviral backbone (FIG. 13A). Semi-quantitative RT-PCR and agarose gel electrophoresis showed a stronger band of the expected size after transfection of the overexpression construct into HL-1 cells. Sanger sequencing of the amplicon revealed correct backsplicing which was further confirmed by resistance to RNaseR exonuclease digestion (FIG. 13B, C). When treated with doxorubicin, HL-1 cells overexpressing circINSR compared with empty vector control showed significantly lower levels of apoptosis concomitant with enhanced cell viability as demonstrated in Annexin V and WST-1 assays, respectively (FIG. 3A,B). Based on the high degree of sequence conservation, next circINSR overexpression was tested in isolated neonatal rat cardiomyocytes (NRCM) and human iPSC-derived cardiomyocytes (hiPSC-CM). To this end, the overexpression cassette was subcloned into an adeno-associated virus (AAV) backbone and packaged into AAV6 serotype particles. Infection of both cell types with AAV6-circINSR induced robust overexpression of the circINSR but had no effect on the linear INSR transcripts (FIG. 13D). Remarkably, overexpression of murine circINSR significantly attenuated doxorubicin-induced apoptosis of NRCMs and hiPSC-CMs as indicated by lower numbers of TUNEL positive cells (FIG. 3C,D). Thus, these results suggest a protective role of circINSR in doxorubicin-induced cardiotoxicity which is conserved amongst mammals including human.

### EXAMPLE 3—CIRCINSR THERAPY PROTECTS FROM DOXORUBICIN-INDUCED CARDIOTOXICITY IN VIVO

To test the potentially beneficial effects of circINSR therapy in vivo, a previously established mouse model of chronic doxorubicin-induced cardiotoxicity was employed^{12, 14}. The AAV-circINSR construct was packaged into AAV9 and injected adult male mice (12 weeks) with a single dose of 2×10\hat{ }12 viral genomes/mouse or with the same amount of AAV9-empty control vectors. After two weeks to allow full AAV mediated gene expression, the mice received a cumulative dose of 25 mg/kg/bw doxorubicin over a course of 5 weeks and were sacrificed 1 week later after echocardiography was performed (FIG. 4A). Indicative of an effective chemotherapy, doxorubicin treatment significantly halted the weight gain observed in control mice. AAV9-circINSR treatment showed a trend towards increased body weight in both doxorubicin and vehicle treated mice (FIG. 4B). Expression analysis confirmed the out previously identified doxorubicin-related downregulation of circINSR (AAV9-empty+/−doxorubicin), while AAV9-circINSR treatment led to a robust upregulation of circINSR expression (FIG. 4C). No significant changes in cardiac expression of the linear hostgene were observed (FIG. 14A). Cardiac function assessed by echocardiography showed a significant reduction of the left ventricular fractional shortening in response to doxorubicin in AAV9-empty controls, but this was fully rescued in mice treated with AAV9-circINSR (FIG. 4D,E). Moreover, the left ventricular volume was increased and cardiac dimensions such as left ventricular anterior and posterior wall thickness were decreased. In line with the ejection fraction, these parameters were significantly improved in mice treated with AAV9-circINSR compared with controls (FIG. 4F-H). Table 1 provides a complete overview of the echocardiography analysis.

| | AAV9-Empty | | | AAV9-circ-Insr | | |
|---|---|---|---|---|---|---|
| | Mean | SEM | N | Mean | SEM | N |
| Heart Rate | 461.2266 | 17.78697 | 6 | 465.1555 | 16.32328 | 6 |
| Diameter; s | 2.43734 | 0.07237817 | 6 | 2.19815 | 0.0601793 | 6 |
| Diameter; d | 3.964799 | 0.06336569 | 6 | 3.800096 | 0.1150373 | 6 |
| Volume; s | 21.16612 | 1.615367 | 6 | 16.32812 | 1.113906 | 6 |
| Volume; d | 68.81789 | 2.591331 | 6 | 62.55784 | 4.541908 | 6 |
| Stroke Volume | 47.65177 | 1.412204 | 6 | 46.22971 | 3.516767 | 6 |
| Ejection Fraction | 69.39774 | 1.370719 | 6 | 73.81739 | 0.7053018 | 6 |
| Fractional Shortening | 38.57322 | 1.048871 | 6 | 42.11384 | 0.6470288 | 6 |
| Cardiac Output | 21.953 | 0.9523026 | 6 | 21.65973 | 2.171054 | 6 |
| LV Mass | 127.2298 | 7.672995 | 6 | 121.5902 | 7.326181 | 6 |
| LV Mass Cor | 101.7839 | 6.138392 | 6 | 97.27218 | 5.860933 | 6 |
| LVAW; s | 1.220428 | 0.07552639 | 6 | 1.138179 | 0.02461471 | 6 |
| LVAW; d | 0.928461 | 0.05735632 | 6 | 1.015078 | 0.03851401 | 6 |
| LVPW; s | 1.130538 | 0.06551825 | 6 | 1.107932 | 0.03588337 | 6 |
| LVPW; d | 0.7836723 | 0.04100587 | 6 | 0.7243488 | 0.0303135 | 6 |
| | AAV9-Empty + Doxo | | | AAV9-circ-Insr + Doxo | | |
| | Mean | SEM | N | Mean | SEM | N |
| Heart Rate | 448.2953 | 10.7211 | 6 | 452.6475 | 16.41001 | 7 |
| Diameter; s | 2.725358 | 0.04590998 | 6 | 2.225428 | 0.08928083 | 7 |
| Diameter; d | 3.958722 | 0.09049105 | 6 | 3.706413 | 0.08749336 | 7 |
| Volume; s | 27.77308 | 1.184302 | 6 | 17.01014 | 1.673801 | 7 |
| Volume; d | 68.58643 | 3.824287 | 6 | 58.77969 | 3.16257 | 7 |
| Stroke Volume | 40.81335 | 2.742513 | 6 | 41.76955 | 1.808686 | 7 |
| Ejection Fraction | 59.3398 | 0.869289 | 6 | 71.41866 | 1.568047 | 7 |
| Fractional Shortening | 31.10078 | 0.6463836 | 6 | 40.07522 | 1.234029 | 7 |
| Cardiac Output | 18.33055 | 1.391195 | 6 | 18.89849 | 1.055651 | 7 |
| LV Mass | 97.95577 | 4.641264 | 6 | 104.0795 | 1.793698 | 7 |
| LV Mass Cor | 78.36462 | 3.713008 | 6 | 83.26357 | 1.434957 | 7 |
| LVAW; s | 0.9483331 | 0.03053827 | 6 | 1.164232 | 0.03440624 | 7 |
| LVAW; d | 0.7865845 | 0.0397983 | 6 | 0.9001749 | 0.06966367 | 7 |
| LVPW; s | 0.832049 | 0.03739992 | 6 | 1.021446 | 0.03834057 | 7 |
| LVPW; d | 0.626641 | 0.02357708 | 6 | 0.7080022 | 0.04360453 | 7 |

In addition to apoptosis, doxorubicin is known to cause cardiomyocyte atrophy¹² (G). Thus, histopathology analyses were performed on transversal heart section. First, co-staining of TUNEL and cardiac troponin T (cTNT) to specifically label apoptotic cardiomyocytes revealed a strong induction of apoptosis in response to doxorubicin which was significantly blunted in mice which received circINSR treatment (FIG. 5A,B). Moreover, cardiomyocyte size measurements on wheat germ agglutinin stained sections showed the expected doxorubicin-mediated atrophic response. However, cardiomyocyte size of mice treated with doxorubicin and circINSR were indistinguishable from control mice (no doxorubicin) (FIG. 5C,D). These data suggest, that circINSR is cardioprotective through mechanisms that include protection from both apoptosis and atrophy.

### EXAMPLE 4—BRCA1 IS INVOLVED IN CIRCINSR FORMATION

In order to gain first mechanistic insight, it was first focused on a potential upstream mechanism that governs circINSR expression. The sequence of the circINSR overexpression construct and the adjacent introns was analysed using a tool for the prediction of splicing regulatory motifs (RegRNA 2.0)¹⁵ and identified a number of potential exon splicing enhancers including BRCA1¹⁶ (FIG. 6A & Table 2).

| Motif Name | Organism | Binding factors |
|---|---|---|
| cftr, exon 9 | Homo sapiens, human | |
| gh exon 5 | Bos taurus, bovine | ASF/SF2 |
| mvm ns2 | Unknown | |
| beta-globin exon 2 | Homo sapiens, human | ASF/SF2 |
| dmd, pseudo exon | Homo sapiens, human | hnRNP G, Tra2β |
| from intron | | |
| bpv-1 late exon 2 | Unknown | SRp75, SRp55, |
| | | SRp30a |
| brca1, exon18 | Homo sapiens, human | SFRS1 (SF2/ASF) |
| cftr, exon12 | Homo sapiens, human | SFRS1 (SF2/ASF) |
| gh-1, exon 3 | Homo sapiens, human | |
| srp40-exonic splicing | Unknown | SRp40 |
| enhancer | | |
| srp55-exonic splicing | Unknown | SRp55 |
| enhancer | | |
| sc35 - exonic splicing | Homo sapiens, human | SC35 |
| enhancer | | |
| srp40 exonic splicing | Unknown | SRp40 |
| enhancer | | |
| sc35 - exonic splicing | Unknown | SC35 |
| enhancer | | |
| srp55 - exonic splicing | Unknown | SRp55 |
| enhancer | | |
| asf/sf2 - exonic splicing | Unknown | ASF/SF2 |
| enhancer | | |

It was particularly focused on BRCA1 since the interaction was predicted within the ALU elements which are known to facilitate circRNA formation¹³. Interestingly, significantly lower levels of Brca1 mRNA expression were found in hearts from doxorubicin treated mice (FIG. 6B). To uncover a potential interaction, siRNAs were designed which efficiently blocked BRCA1 mRNA and protein expression in HL-1 cells (FIG. 15A,B). Indeed, inhibition of BRCA1 led not only to decreased expression of endogenous circINSR, but also to lower amounts of circINSR upon overexpression (FIG. 6C,D). Strikingly, the expression of the linear INSR mRNA was not affected, therefore, suggesting hostgene-independent regulation of circINSR that involves BRCA1.

### EXAMPLE 5—CIRCINSR IMPACTS ON SIGNALING PATHWAYS RELATED TO CARDIAC DEVELOPMENT AND PHYSICALLY INTERACTS WITH SSBP1

To gain first insight into the mechanisms and molecular pathways underlying the cardioprotective effects of circINSR, total RNA sequencing was performed in HL-1 cells after silencing and after overexpression of circINSR (siRNA scramble vs. siRNA circINSR and empty vector vs. circINSR vector, respectively). More than 500 genes in both comparisons were differentially expressed. 23 genes overlapped and were reciprocally expressed after silencing or overexpression of circINSR which was confirmed for Wnt6 as an exemplary gene by RT-qPCR in independent samples (FIG. 7A, B). Basic pathway analysis with these 23 genes using the EnrichR tool identified several cardiac-related pathways, the most enriched ones being “Heart Development mouse/human” and “Wnt signalling and Pluripotency” (FIG. 7C). In line with these data it was found that silencing circINSR in human iPSC, leads to loss of pluripotency as indicated by dramatic reduction of Oct4, Nanog and Sox2 mRNA expression (FIG. 16A). Interestingly, in addition to developmental processes, Wnt signalling is also activated during cardiac remodelling and the progression to heart failure¹⁷. Further detailed gene set enrichment analysis (GSEA) was performed using the Broad Institute GSEA software package^{18, 19} There were three KEGG gene sets reciproally enriched after circINSR overexpression or knock-down. These data suggest that circINSR is functionally involved in cellular processes such as ribosome, spliceosome and DNA replication mechanisms (FIG. 7D).

Finally, RNA pull-down experiments were performed to identify potential protein interaction partners of circINSR. To do so, a biotin-labelled probe was designed which spans the backsplice junction (FIG. 8A). Validation experiments by RT-qPCR showed that the probe specifically enriched circINSR but notthe linear hostgene and vice versa for a linear hostgene-specific probe (FIG. 17A,B). Mass spectrometry analysis of the pull-down lysates yielded a list of potential protein interactors. After applying several filtering parameters (relative MS peptide count >15, relative enrichment over hostgene and control probe log 2FC>2) and cat rapid analysis to calculate the interaction propensity with circINSR, Single-stranded DNA-binding protein 1, SSBP1, was identified as the most probable binding partner of circINSR (FIG. 8B). Independent pull-down experiments with the circINSR probe followed by Western blotting with SSBP1-specific antibodies confirmed a physical interaction (FIG. 8C). Moreover, co-localisation of circINSR and SSBP1 in Immuno-RNA-FISH staining, further confirmed the interaction between those two.

### EXAMPLE 6—CIRCINSR IS RELEASED INTO THE EXTRACELLULAR SPACE IN EXOSOMES AND MICORVESICLES

NcRNAs can be released into the extra-cellular space through passive mechanisms (e.g. necrosis of cells) or actively within secreted microvesicles²⁰. Since they can be readily isolated and measured from body fluids such as blood, these circulating ncRNAs have gained much attention as potential biomarkers for various diseases^{21, 22}. Indeed, ot was possible to detect circINSR in human and murine plasma (FIG. 9A,B). Strikingly, levels of circulating circINSR were diminished in mice treated with doxorubicin, which warrants further investigation of circINSR as a marker for anthracycline-related heart failure. Then the presence of circINSR in exosomes isolated from HL-1 cell culture supernatants was tested. CircINSR was detectable in both the microvesicle and the exosome isolates (FIG. 9C), suggesting that circINSR is actively released from cardiomyocytes. To further corroborate the findings, a circINSR knock-out cell line was generated. Using a dual guideRNA CRISPR/Cas9 approach the majority of the circINSR forming exon was excised (FIG. 18A,B). Expectedly, this not only abrogated circINSR expression but also INSR protein expression (FIG. 18C,D). Then this cell line was used to overexpress circINSR via transfection with pcDNA3.1. As shown in FIG. 9D, only a few cells carry the overexpression construct indicated by GFP expression. However, RNA-FISH experiments detected circINSR not only in GFP expressing cells but also in GFP negative cells, suggesting that circINSR can be transferred between cells, presumably via exosomes.

### EXAMPLE 7—CIRCINSR CAN BE PRODUCED BY IVT AND PROTECTS FROM DOXORUBICIN-INDUCED TOXICITY

To circumvent the use of viral gene therapy vectors, the direct use of RNA produced by in vitro transcription (IVT) has been explored for some linear RNAs²³. Thus, it was asked whether circINSR may be formed by IVT. To do so, the circINSR including the adjacent ALU elements was cloned in front of a T7 promoter and RNA was produced using a commercially available IVT kit (FIG. 10A). RT-qPCR using divergent primers demonstrated that circularization of circINSR occurs in vitro (FIG. 10B). Transfection of HL-1 cells with the IVT products resulted in higher levels of circINSR and pre-circINSR (non-circularized) but not in the endogenous mRNA levels of INSR (FIG. 10C). Strikingly, adding the circINSR IVT product to doxorubicin treated HL-1 cells, partially rescued the induction of apoptosis and increased cell viability (FIG. 10D,E). Finally, since ectopically applied RNA may induce immune responses, the IVT products were transfected into RAW264.7 macrophages. RT-qPCR experiments showed a strong increase in circINSR, however, genes indicative of immune responses (TNF-a, CXCL1 and CXCL12) remained unchanged (FIG. 19A,B).

### EXAMPLE 8—CIRC-INSR EXPRESSION IN MOUSE ACUTE CARDIAC ISCHEMIA-REPERFUSION INJURY (I/R INJURY) MODEL

It is reported that human circ-Insr was found to be highly enriched in hiPSC-CMs as compared to other human cells such as HEK-293 Ts, HCFs, HUVECs and hiPSCs (FIG. 20A). To further investigate circ-Insr in other kinds of cardiac vascular diseases (CVDs), circ-Insr expression was tested in a mouse acute cardiac ischemia-reperfusion injury (I/R injury) model. Circ-Insr significantly decreased in infarct region of I/R injury heart while no change was observed in remote region compare to sham group (FIG. 20B). Furthermore, the expression of circ-Insr was evaluated in HL-1 cells treated with other kinds of anthracyclines including epirubicin, daunorubicin and paclitaxel. Here, significant downregulation of circ-Insr was observed in HL-1 cells under epirubicin and daunorubicin stress compare to control, respectively. And no change showed in paclitaxel treated cells (FIG. 20C-E). Collectively, these data extend our report that circ-Insr is a key regulator in human cardiomyocytes and could regulate I/R injury and anthracyclines (epirubicin and daunorubicin) induced cardiotoxicity.

### EXAMPLE 9—MITOCHONDRIAL STRESS TEST IN CIRC-INSR SIRNA TREATED NEONATAL MOUSE CARDIOMYOCYTES (NMCM)

The above examples, demonstrate that circ-Insr is necessary for cardiomyocyte survival, especially regulating doxorubicin mediated cardiomyocytes apoptosis. Mitochondrial function is also a key parameter which gets affected during doxorubicin-induced cardiotoxicity and, hence, a mitochondrial stress test was applied in circ-Insr siRNA treated neonatal mouse cardiomyocytes (NMCM) compared to the scramble siRNA. Mitochondrial respiration reflected by OCR levels downregulated in circ-Insr knockdown NMCM compare to control (FIG. 21A). Additionally, lower levels of circ-Insr in NMCM also induced the reduction of non-mitochondrial oxygen consumption, proton leak, and spare respiratory capacity in basal condition (FIG. 21B-E). Interestingly, we also observed decrease in mitochondrial biogenesis-related genes (Pgc1-α, Nrf2a, Tfb2M, Polg and Top1mt) in circ-Insr knockdown neonatal rat cardiomyocytes (NRCM) (FIG. 21F). Collectively, these data demonstrated that loss of circ-Insr affected metabolic activity in cardiomyocytes.

### EXAMPLE 10—MITO-STRESS ASSAY IN NMCM TREATED WITH AAV6-CIRC-INSR

To further investigate whether overexpression of circ-Insr in NMCM could rescue metabolic activity impaired by doxorubicin, mito-stress assay was applied in NMCM treated with AAV6-circ-Insr compare to AAV6-Empty under basal condition or doxorubicin (1 μM and 5 μM, 2 h) stress. OCR level was significantly rescued by circ-Insr overexpression with the treatment of both high and low doses of doxorubicin compare to control (FIG. 22A). Likewise, circ-Insr could enhance the cell metabolism such as basal respiration, maximal respiration, and spare respiratory capacity and ATP production under basal condition. Importantly, maximal respiration and spare respiratory capacity were preserved by circ-Insr overexpression after treatment with both high and low doxorubicin concentrations, whereas the recovery of basal respiration and ATP production were only observed under high doxorubicin concentration (FIG. 22B-E). Furthermore, we observed that mitochondrial biogenesis-related genes (Pgc1-α, Nrf2a, Nrf2b, Tfb1m, Tfb2m and SSBP1) were enhanced by circ-Insr overexpression in NRCM (FIG. 22F). These results suggested a protective role of circ-Insr in doxorubicin-induced cardiotoxicity and the metabolic disorder.

### EXAMPLE 11—INTERACTION BETWEEN CIRC-INSR AND SSBP1

Based on the results of the foregoing examples, it was reasoned that circ-Insr could interact with SSBP1. To further confirm the interaction between circ-Insr and SSBP1, siRNA mediated circ-Insr knock-down was applied in HL-1 cells. Next, RNA pulldown was performed with circ-Insr and scramble biotinylated probes. RT-qPCR confirmed that decrease of circ-Insr appeared both in input and circ-Insr pulldown group (FIG. 23A). Interestingly, the protein level of SSBP1 was not regulated in total cell lysates which was transfected with circ-Insr siRNA, while the decreased levels of SSBP1 were observed only in pulldown group (FIG. 23B). SSBP1 is known to regulate mtDNA stability (Kaur P, Longley M J, Pan H, Wang H, Copeland W C. Single-molecule DREEM imaging reveals DNA wrapping around human mitochondrial single-stranded DNA binding protein. Nucleic acids research. 2018; 46:11287-302.) and replication (Morin J A, Cerron F, Jarillo J, Beltran-Heredia E, Ciesielski G L, Arias-Gonzalez J R, et al. DNA synthesis determines the binding mode of the human mitochondrial single-stranded DNA-binding protein. Nucleic acids research. 2017; 45:7237-48). Therefore, we assumed that circ-Insr is a cofactor of SSBP1 resulting in the regulation of mitochondrial function in cardiomyocytes by stabilizing mtDNA. Thus, mtDNA copy number was measured using RT-qPCR indicated that doxorubicin significantly reduced mtDNA content, while overexpression of circ-Insr could reverse the decrease of mtDNA copy number regulated by doxorubicin (FIG. 23C). Notably, circ-Insr overexpression can also significantly reduce percentage of cells undergoing mitochondrial fission compared to negative control under doxorubicin exposure (FIG. 23D). Next, we constructed SSBP1-flag overexpression plasmid and WB indicated that both flag-tag and SSBP1 were enhanced in HL-1 cell (FIG. 23E). Furthermore, mito-damage assay was applied to measure the changes in mitochondrial membrane potential (MMP). We observed that doxorubicin could impair MMP, while circ-Insr siRNA could further increase the damage of MMP compared to scramble siRNA under doxorubicin stress. Markedly, SSBP1 overexpression could block the further damage effect of MMP under doxorubicin treatment which leads to endogenous knockdown of circ-Insr (FIG. 23F). Next, we co-transfected HL-1 cells with pcDNA-circ-Insr and SSBP1 siRNA and examined cell viability using the WST assay to explore whether SSBP1 participated in the circ-Insr overexpression-mediated therapy of doxorubicin-cardiotoxicity. RT-qPCR indicated SSBP1 siRNA could downregulate endogenous SSBP1 expression (FIG. 23G). As observed before, doxorubicin could impair HL-1 cell viability and overexpression of circ-Insr could enhance the cell survival under doxorubicin treatment. Interestingly, SSBP1 siRNA blocked the rescue effect mediated by circ-Insr overexpression under doxorubicin stress (FIG. 23H). Collectively, these data demonstrated that SSBP1 is a critical target gene of circ-Insr in regulating cardiomyocyte apoptosis and mitochondrial function.

### EXAMPLE 12—CONCLUSION

In this report, we summarized that circ-Insr is enriched in human cardiomyocytes compare to other human cardiac cell types. In addition, circ-Insr is also regulated in I/R injury and anthracyclines (epirubicin and daunorubicin) induced cardiotoxicity. Despite the regulation of cardiomyocytes apoptosis, circ-Insr could also affect mitochondrial functions (mitochondrial biogenesis-related genes, mtDNA content, mitochondrial fission and mitochondrial membrane potential) and metabolic activity in cardiomyocytes. Notably, SSBP1 is identified as a binding target of circ-Insr and participates in the doxorubicin induced cardiotoxicity therapy mediated by circ-Insr overexpression.

### EXAMPLE 13—METHODS

### Animal Experiment:

C57BL/6 N mice of 8 weeks of age were injected (intraperitoneally) with doxorubicin at a dose of 5 mg/kg once a week for consecutive 5 weeks. One week later after the last doxorubicin treatment, echocardiography data were recorded by an independent blinded researcher using the Vevo 2100 system (Fujifilm Visulasonics, Inc). Mice were euthanized, and hearts were harvested and processed for further molecular and cellular assays. Echocardiography data were analyzed using standard imaging protocols (M-mode and B-mode) for global cardiac volumes and functioning using the Vevostrain software (Fujifilm Visulasonics, Inc). Animal experiments were approved by the local authorities at Hannover Medical School and Niedersachsen Landesamt für Verbraucherschutz (Animal license lumber: TVA14/1665). AAV9 (adeno-associated virus serotype 9) was injected (intravenously) 2 weeks before first doxorubicin injection. Animal experiments were randomized and blinded with an internal number.

### Cell Culture:

HL-1 cells were cultured in Claycomb medium together with 10% FBS, norepinephrine, L-Glutamine and pencillin/streptomycin as manufacturer's protocol. 3T3 and RAW264.7 cells were cultured in DMEM with 10% FBS and pencillin/streptomycin. Neonatal rat cardiomyocytes (NRCM) were isolated from one to three-day old rat babies by using the Neonatal Heart Dissociation Kit (Miltenyi). Human iPSC derived cardiomyocytes (hiPSC-cm) were differentiated at the lab of IMTTS. All the cells were treated with 0.25 μM Doxorubicin (Sigma-Aldrich) for 48 hours. circ-Insr siRNA (Eurofins), in vitro circular RNA and overexpression plasmid were transfected with Lipofectamine 2000 (Life Technologies) in OptiMEM medium. NRCM and hipsc-CM were transduced by AAV6 (MOI: 1*10\hat{ }4) for 72 hours.

siRNA Sequence:

Scramble siRNA:
(SEQ ID NO: 19)
AGG UAG UGU AAU CGC CUU G
Circ-Insr siRNA1:
(SEQ ID NO: 20)
GAA AGU GUG CCC UGG UAU G
Circ-Insr siRNA2:
(SEQ ID NO: 21)
GUC AUU GUC AGA AAG UGU G

### WST Assay:

WST-1 assay was performed using Cell Proliferation Reagent WST-1 (Roche) as per the manufacturer instruction. Briefly, HL-1 cells were seeded in 96 well plate and next day treated with 0.25 μM Doxorubicin in normal medium with 10% FBS for forty-eight hours. Next, 10 μL WST1 reagent was added and incubated for 1 hour followed by the absorbance measurement (450 nm) at HT Synergy (Biotek) plate reader.

### Annexin V & 7-AAD Staining:

FlowCellect Annexin Red kit (Millipore) was used to stain apoptotic cells as per the manufacturer instruction. Briefly, cells were seeded in 12 well plate and next day treated with 0.25 μM Doxorubicin in normal medium with 10% FBS for forty-eight hours. Then cells were trypsinized and harvested and stained with Annexin-V for 15 minutes at 37° C. in 1× assay buffer. Annexin-V stained cells were centrifuged and washed with 1× assay buffer. Next cells were incubated with 7-AAD for 5 minutes and acquired on Guava (Millipore) flow cytometer. Data was analyzed with FLOWJO software.

### TUNEL Staining:

Cells were fixed with 4% paraformaldehyde for twenty minutes at room temperature and then permeabilized with ice-cold 0.1% Triton-X-100 in PBS for two minutes at room temperature. Next cells were incubated with enzyme labelling solution provided with In Situ cell death detection kit (Roche) for 1 hour at 37° C. For negative staining, enzyme was not added to the labelling solution. Cells were then washed and incubated with Dapi for 15 minutes. To specifically label neonatal rat cardiomyocytes and hipsc-CM, cells were co-stained with cardiac Troponin T (Thermofisher) after Tunel staining. Images were taken with Nikon Eclipse Ti microscope and images were analyzed with NIS Elements. For analysis in each case, ten different images were analyzed from different regions and average value was taken. Similarly, cryosections of heart were also processed for staining. Numerous images covering complete heart section was taken and Tunel positive nuclei were counted in each field. Average value for each heart was calculated.

### Cell Size Measurement

Cryosections of heart were stained with Alexa Fluor 488 labelled wheat germ agglutinin (Invitrogen) and dapi. Images were taken with Nikon Eclipse Ti microscope and images were analyzed with NIS Elements. For each heart 5 to 10 images were randomly taken from different regions of the section and minimum two hundred cells were measured and average value was taken for each heart.

### Immunostaining

3T3 cells were fixed with 4% paraformaldehyde for ten minutes and permeabilzed with 0.1% Triton-X-100 for 10 minutes. Blocking was done for thirty minutes with donkey serum and then incubated with INSR antibody (Abcam) (1:300) at 4° C. overnight. Next day, cells were incubated with secondary antibody Anti-Rabbit-Alexa 594/488 (Invitrogen) and dapi for 30 minutes. Images were taken with Nikon Eclipse Ti microscope and images were analyzed with NIS Elements.

### RNA Isolation & PCR

RNA isolation from cell culture and heart tissue was done using Trifast (Peqlab) as per the manufacturer's instructions. Isolated RNA (500 ng-1000 ng) was reversed transcribed with random primer using iScript Select cDNA synthesis kit (Biorad). Real-Time quantative PCR was done with iQ SYBR Green mix (Biorad) on C1000 Touch Thermocycler (Biorad) using specific primer pairs. For amplification of circular RNAs, divergent primers were used while normal linear transcripts were amplified by convergent primers as usual. For RNase R resistant assay RNA was incubated with RNase R (Biozym Scientific) at 37° C. for 10 minutes and heat activated at 95° C. for three minutes. RNA was then reverse transcribed and amplified by specific PCR primers as mentioned before.

Primer sequences (5′→3′):

Circ-Insr (mmu/rno/hsa):
Fwd
(SEQ ID NO: 22)
CTGTCCTGCCACTGTCATCAA
REV
(SEQ ID NO: 23)
TCTTCGGGTCTGGTCTTGAAC
linear-Insr (mmu/rno/hsa):
Fwd
(SEQ ID NO: 24)
TCACCTGAGTCCCTGAAGG
REV
(SEQ ID NO: 25)
GCATCAGGTCAGTGAGTCTC
Pre-Insr (mmu/rno/hsa):
Fwd
(SEQ ID NO: 26)
GATCCATCTATCTCTAAATGCCC
REV
(SEQ ID NO: 27)
TCTTCGGGTCTGGTCTTGAAC
18s (mmu/rno):
Fwd
(SEQ ID NO: 28)
GTAACCCGTTGAACCCCATT
REV
(SEQ ID NO: 29)
CCATCCAATCGGTAGTAGCG
18s (hsa):
Fwd
(SEQ ID NO: 30)
AGTCCCTGCCCTTTGTACACA
REV
(SEQ ID NO: 31)
GATCCGAGGGCCTCACTAAAC
Xist (mmu):
Fwd
(SEQ ID NO: 32)
TGCCTGGATTTAGAGGAG
REV
(SEQ ID NO: 33)
CTCCACCTAGGGATCGTCAA
WNT6 (mmu):
Fwd
(SEQ ID NO: 34)
GCAAGACTGGGGGTTCGAG
REV
(SEQ ID NO: 35)
CCTGACAACCACACTGTAGGAG
Brca1 (mmu/rno)
Fwd
(SEQ ID NO: 36)
ATCTGCCGTCCAAATTCAAG
REV
(SEQ ID NO: 37)
TTCCAAACAGATCGGACACTC
SSBP1 (mmu/rno)
Fwd
(SEQ ID NO: 38)
CAACAAATGAGATGTGGCGATCA
REV
(SEQ ID NO: 39)
ACGAGCTTCTTACCAGCTATGA

### Western Blotting

Cell pellets were lysed in 1× Cell lysis buffer (Cell Signaling) and isolated protein was measured by Bradford (Biorad) for quantification. 15-30 μg of protein was loaded for each sample on SDS-polyacrylamide gel to resolve the protein. Proteins were transferred from SDS PAGE gel to polyvinylidene fluoride membrane in Mini PROTEAN Tetra cell (Biorad). Specific proteins were identified by following antibodies: BRCA1 (Sigma), SSBP1 (proteintech) and Vinculin (Sigma). HRP conjugated secondary antibody (Cell Signalling) was used for detection of bands. Band intensity was calculated by Image J software.

### Circular RNA Overexpression Strategy:

Circ-Insr overexpression transcript was cloned from HL-1 cell gDNA by using HotStarTaqMasterMix (Qiagen). EcorI and XhoI were included in the forward and reverse primer for the pcDNA3.1 plasmid cloning and EcorI and SaLI were used as the restriction enzyme sites for the AAV plasmid cloning.

The circ-Insr overexpression transcript sequence:

(SEQ ID NO: 40)
ctcactggggaattctatgaagggctatgtaccctcactgggagatctaggcaggggttctacagctgaacagtgtcctcctgtg
acacaggttatctttaaactcataatcctcctgcctcagcctctttatcaatggagtttaagtggtcaccatgcctagcacatcatatatc
attatctgtcatccacagttagggcaatggctggtatcataaagacaagaagtaacaaatactagtgagaatattaaggaagggg
agcgtttacatactgttaacaaccaggaagattgaaaaggagtcctagttctttttcttcctcaagtcttttggttgacttatagacgaat
gggtttcacgcgatgtcatcttatgtgcatcgatcgtaccttgtttatcacctctgctgcattttctgcactcagctctctgctcacccatta
gtatctttctttactcaactggtctctcttctgttcctgccgtgcacatatacagtgcatatatatctaattatttcattctttctggctactac
cccattgtttctatatcccacatttttgatcttatctcttattattattactttcttgagacagggtctcaaatagcctaggcaggcatagtctag
ctcccacccattgaccgcttatttcataacttggttattgtgaagatgcctctatatgatatgtgccacattttcttcatctgtttttctgttggc
agacatttcctcagctgtggtgaacagagcaacagtgagagtagatgtgcagtggagtgtctgtgatatgctgacctagagtgctctg
ggacatacacccctggttttttttttttttttttgtgtgtgtgtgagaagccaagaccaatttcaagggttagttgttcctcaggtgccctccat
cttgtctcttgagacagggtctcttactggcctggagttcactaatagaagtagcccagtgagccctggggatccatctatctctaaat
gccccctgccctcacagacatattatgttacacaagcttattacatgggtcctgggcatagatgctaggccctcatccctgtacaac
actttgttgtctccccagttctaggttctagttccgttagccgttctagagtccctggctcacctactttgctttgcctttattatctgccttcta
gtgtgccctggtatggacatccggaacaacctgaccaggctacatgagctggagaactactcagtcattgagggccatctccag
atcctcctgatgttcaagaccagacccgaagatttccgagacctcagtttccccaaactcatcatgatcacagattacctgcttctctt
ccgtgtctatggtctggaaagtctgaaagacctcttcccaaatctcacagtcatccgaggctcccgtctcttcttcaactatgccctggt
tatcttcgagatggtccacctgaaggagctggggctttataacctcatgaacatcacccggggctcttccgcatcgagaagaata
atgagctctgctacctggccactatcgactggtcccgtatcctggattctgtggaggacaactacattgtactgaacaaagatgaca
acgaggaatgtggggatgtctgtccaggcaccgccaagggcaagaccaactgtcctgccactgtcatcaatgggcagtttgtgg
aacggtactggacacacagtcattgtcagaaaggtatgctgaaggcagtgccttttaaagatttctccgtggttagtatgcccagga
gaagtagactcctgtaaaagtttaggtgatggaggttgtagggtacaaaccctagaatctccataaaaaatacattcgtccccttat
ctgcgaggcatacctatttgatgttgtacacttaactgtgggttactgagactgtaggaagcagaatttctgatggggagagatgaat
gcgtgtactgatacttggccattgcagtaggaaggtagccacagttaggtatcaagatatggtgttttttttttaatgtggcttatatgtaa
aaaaatttttatatgcaaaaaataaatagggggccagatttcttttgggggagagctgtttgccaccctctgtgctggatagtgactgt
ttctgtggtcctctgctttgttggggagtagactatgcaagagtatgggtttggcttagtgtttttcttatctgatcatagaagtcaatttgtta
gagatactcagaaaaagaaatgtaaaaattcagctttgaccgggtgtggtggcgcacgcctttaatcccagcactcgggaggca
gaggcaggtagatttctgagttctaggccagcctgttctacagagtgagtttcaggacagccagggctatatagtgaaaccctgtct
caaaaaaccaaaaaaccaaaacaaacaaacaaaaaaaccccagctttgatgagggtagct

The underlined regions are circularization elements for circ-Insr, double underlined region is the exon sequence of circ-Insr, bold and italic regions are primer binding sequences.

Cloning primer sequence (5′→3′):
For pcDNA3.1 Plasmid System:

(SEQ ID NO: 41)
Ctcactggggaattctatgaagg (Fwd with EcorI)
(SEQ ID NO: 42)
CCGCTCGAGTagctaccctcatcaaagctg (Rev with xhoI)

### For AAV Plasmid System:

(SEQ ID NO: 43)
Ctcactggggaattctatgaagg (Fwd with EcorI)
(SEQ ID NO: 44)
CCGTCGACTagctaccctcatcaaagctg (Rev with SalI)

### Adeno-Associated Virus Production

HEK 293T cells were transfected with AAV-Empty or AAV-circ-Insr plasmid together with pDG (a kind gift from Prof. Roger Hajjar, Mount Siani Hospital, New York) (pDG2 for AAV2 and pDG9 for AAV9) with polyethylenimine. Medium was changed next day and cells were left for seventy two hours. Supernatant was collected and mixed with 40% Polyethylene Glycol 8000 solution for an hour and later incubated overnight at 4° C. Cells were harvested and lysed in presence of benzonase (Novagen). Supernatant was centrifuged at 2800×g for 15 minutes to collect precipitated virus and mixed with the cell lysate. Cell lysate was ultracentrifuged on iodixanol (OptiPrep, Progen) gradient in ultracentrifuge (Beckman Coulter) at 63000 rpm for one hour in Ti 70 rotor. AAVs were extracted from 40% fraction and concentrated using Amicon Ultra 15 (Millipore) columns. AAVs were titrated with PCR primer amplifying CMV (Cytomegalovirus) promoter.

### CRISPR Cas9-Mediated Circ-Insr Knockout:

The sgRNA was designed via website tool (http://crispr.cos.uni-heidelberg.de/) with the core settings of max mismatch=3, PAM=NGG-NRG. The target sequences are listed here:

sgRNA1:
(SEQ ID NO: 45)
ACGGAACTAGAACCTAGAACTGG;
sgRNA2:
(SEQ ID NO: 46)
GTCCTGCCACTGTCATCAATGGG.

gRNA1 was first sub-clone in pL40C backbone (hU6) and gRNA2 in pSGL40C (mU6). Then, excise sgRNA with mU6 promoter form the pSGL40C with EcoRI/XhoI digest and re-ligase gRNA2 to the gRNA1-pL40C plasmid. After the Dual CRISPR plasmid is generated, 500 ng plasmid was transfected to the 3T3 cells. 72 hours later, single cell was sorted according to RFP signal utilizing MoFlo XDP Cell Sorter (beckman). Genotyping were performed by using the primer list below.

### Checking Primer:

Fwd:
(SEQ ID NO: 47)
cggggctggcttaagagttt
Rev:
(SEQ ID NO: 48)
agtttgatcctctatgcccac

In Vitro Transcription of circRNA:

In vitro circ-Insr was produced using RiboMax large RNA production system (Promega) according to the manufacturer's protocol with slight modifications. Briefly, 500 ng PCR-amplified T7-circ-Insr overexpression DNA fragments were incubated with 1 μL T7 RNA polymerase enzyme mix, 1 mM GTP and 5 mM each of other NTPs. The reaction was carried out for 2 hr at 37° C., followed by DNase I treatment for 30 min at 37° C. to remove DNA templates. Transcribed RNAs were precipitated by miRNeasy Mini Kit (Qiagen) and eluted in RNase-free water.

### RNA Fish:

Circ-Insr in situ hybridization (ISH) was performed by using the ViewRNA Cell Plus Assay (Thermo Fisher) in HL-1 and 3T3 cells. In brief, HL-1 and 3T3 cell were fixed and permeabilized by using the buffer in kit. Circ-Insr specific probe were first harvested overnight at 4° C. Following with the Amplifier mix incubation, the signal of RNA were amplified. Images were taken with Leica confocal microscope and images were analyzed with Image J.

### RNA Pull-Down:

10\hat{ }6 cells were washed in ice-cold phosphate-buffered saline, lysed in 500 μl co-IP buffer, and incubated with 3 μg biotinylated DNA oligo probes against endogenous or ectopically expressed circ-Insr and linear-Insr, at room temperature for 2 h. A total of 50 μl washed Streptavidin C1 magnetic beads (Invitrogen) were added to each binding reaction and further incubated at room temperature for 2 hour. The beads were washed briefly with co-IP buffer for five times. The bound proteins in the pull-down materials were analyzed by western blotting and the RNA sample were analysed via qRT-PCR.

Anti-linear-Insr probe:
(SEQ ID NO: 49)
5′ Biosg-AAAATTAGACAGGCCTTGATAAGGTTGCTCAGC-3'
Anti-circ-Insr probe:
(SEQ ID NO: 50)
5' Biosg-AAACCATACCAGGGCACACTTTCTGACAATGAC-3'

### Mass Spectrometry Analysis:

RNA pull-down lysates were acquired following the method mentioned in RNA pull-down. The protein lysate was alkylated using acrylamide (40%, 4K solution; Applichem, Darmstadt) for the purpose of mass spectrometry analysis. 50 μl RNA pull-down sample of each lysate were loaded onto 4-15% Mini-PROTEAN TGX Precast Protein Gels (Bio-Rad) and separated using SDS-PAGE. Proteins were stained using Coomassie Brillant Blue G250 (Thermo Fisher) according to standard procedure. The stained SDS-gel was further processed for mass spectrometry analysis at the MS Core Facility Proteomics at the Hannover Medical School. Therefore, the proteins samples in the designated range were in-gel digested and the harbouring peptides were analysed using the LC-MS System (Thermo Fisher Scientific).

### Exosome and Microvesicle Isolation:

Exosomes were purified from conditioned medium of neonatal rat cardiac fibroblasts cultured in DMEM supplemented with 1% exosome-depleted FBS (FBS is depleted of contaminating bovine exosomes by ultracentrifugation for at least 6 hours at 100,000 g). Conditioned medium of rat cardiac fibroblasts was collected for 48 hours, and exosomes were purified by several centrifugation and filtration steps. Briefly, the supernatant was centrifuged at 300 g for 10 minutes, 2,000 g for 10 minutes, and 10,000 g for 30 minutes, followed by filtration through a 0.22-μm filter to eliminate cells, dead cells, and cellular debris. For exosome purification, the supernatant was ultracentrifuged at 100,000 g for 70 minutes, followed by an additional washing step of the exosome pellet with PBS at 100,000 g for 70 minutes (Ultracentrifuge, Beckman Coulter, L8-70M). The exosome pellet was resuspended in 100 μl PBS and stored at −80° C.

### Statistics:

All data were analyzed using GraphPad Prism software. Data are presented as mean±SEM, and an unpaired 2-tailed t test was performed to calculate significance between 2 groups, and 1-way ANOVA with post hoc Tukey test was used to calculate significance difference between ≥3 groups wherever required.

### SSBP1-Flag Overexpression Plasmid Construction:

SSBP1-flag transcript was synthesised by GeneArt (Thermo fisher). XbaI and BamHI were included in the transcript. To clone the SSBP1-flag plasmid, we first double digested the lentiCRISPR v2 (addgene: #52961) and then collected the backbone with EF1α-promoter. Next, SSBP1-flag transcript was ligated to the lenti-backbone. The SSBP1-Flag overexpression transcript sequence:

(SEQ ID NO: 57)
GAATTCTCTAGAGCCACCATGTTTCGAAGACCTGTGTTACAGGTATTTC
GTCAGTTTGTAAGACATGAGTCTGAAGTAGCCAGCAGTTTGGTTCTTGA
ACGATCTCTGAATCGTGTTCAGTTACTTGGACGAGTAGGTCAGGACCCT
GTCATGAGACAGGTGGAAGGAAAAAACCCAGTCACAATATTTTCTCTAG
CAACAAATGAGATGTGGCGATCAGGGGATAGTGAAGTATACCAAATGGG
TGACGTTAGTCAGAAGACGACGTGGCACAGAATATCAGTGTTTCGACCA
GGCCTCAGAGATGTGGCATATCAGTATGTGAAAAAGGGGGCTCGTATAT
TTGTGGAAGGGAAAGTGGACTATGGCGAGTACATGGATAAAAACAATGT
GAGGCGGCAAGCAACAACAATCATAGCTGATAACATTATATTTCTGAGT
GACCAGACAAAAGAAAAGGCAGATTACAAGGATGACGACGATAAGGACT
ATAAGGACGATGATGACAAGGACTACAAAGATGATGACGATAAATAGG
GATCC

The double-underlined region is the sequence of Flag-tag, the underlined region is the CDS sequence of SSBP1, italic (XbaI) and bold (BamHI) regions are restriction enzyme sites.

mtDNA Copy Number Measurement:

The ratio of mtDNA to genomic DNA was calculated by dividing copies of CYTB with copies of ACTB in each experiment (Li J, Chan M C, Yu Y, Bei Y H, Chen P, Zhou Q L, et al. miR-29b contributes to multiple types of muscle atrophy. Nature communications. 2017; 8). Each 10 ml reaction contained 2.0 ng of DNA extract, SYBR green mix and 100 nM of each primer. Reactions were performed using a real-time PCR system: 95_C for 10 min, followed by 45 cycles at 95° C. for 10 s, 55° C. for 15 s and 72° C. for 28 s. Fluorescence was measured during the last step of each cycle using the SYBR channel. The primers used are as followed:

Mt-CYTB forward primer:
(SEQ ID NOs 58 and 59)
5-GGCTACGTCCTTCCATGAGGAC-3,
reverse primer:
5-GAAGCCCCCTCAAATTCATTCGAC-3;
ACTB forward primer:
(SEQ ID NOs 60 and 61)
5-CATCTCCTGCTCGAAGTCTAG-3,
reverse primer:
5-ATCATGTTTGAGACCTTCAACACCC-3.

### Seahorse Extracellular Metabolic Flux Assay:

NMCM were plated at 15,000 cells per well in XF96 cell culture microplates coated with 1% Gelatine. The bioenergetics response of NMCM was measured with the Seahorse XFe96 Analyzers following with the manufacturer's protocol of the XF Cell Mito Stress Test Kit. Media was aspirated and replaced with 200 μL assay medium (Agilent) and pre-equilibrated for 1 h at 37° C. Baseline OCR measurements were performed followed by injection of 1 μM of oligomycin with three OCR measurements, then injection of 1 μM FCCP with three OCR measurements, and finally injection of 0.5 μM of rotenone and antimycin A followed by three OCR measurements. For doxorubicin treatment, 1 μM and 5 μM doxorubicin were first injected into well and incubate for 2 h via the free port from XFe96 sensor cartridges before the oligomycin injection. After measurement, NMCMs were stained with hoechst 33342 (Thermo fisher) and performed cell number analysis via Cytation 1 (Biotek). The cell numbers were utilized as the reference to normalize seahorse results.

### Analysis of Mitochondrial Fission:

Mitochondrial fission was analysed by staining mitochondria via MitoTracker® Deep Red FM (Cell Signalling) (Aung L H H, Li R B, Prabhakar B S, Maker A V, Li P F. Mitochondrial protein 18 (MTP18) plays a pro-apoptotic role in chemotherapy-induced gastric cancer cell apoptosis. Oncotarget. 2017; 8:56582-97). Briefly, HL-1 cells were plated onto the Lab-Tekil chambered #1.5 German Coverglass system (Thermo fisher). After treatment, they were stained for 15 min with 100 nM MitoTracker® Deep Red FM (Cell Signalling) and Hoechst 33342 (Thermo fisher). Cells were fixed in 4% paraformaldehyde for 15 minutes. Mitochondria were imaged using Nikon Eclipse Ti microscope with 60× oils Objective (CFI Plan Apo Lambda 60× Oil) and images were analyzed with NIS Elements. To quantitatively analyse cells with mitochondria fission, those cells with disintegrated mitochondria were considered as fission. The total cell numbers were counted via Hochest 33342. The percentage of cells with fission mitochondria relative to the total number of cells was presented as the mean±SEM of at least three independent experiments. A blinding image counting was applied in this experimental. More than 150 cells in 10-15 random fields were counted.

### Primer List: (SEQ ID NOs 62 to 91)

| Primer name | Primer sequencing |
|---|---|
| Rno-PGC1-alpha-F | GGAGCAATAAAGCAAAGAGCA |
| Rno-PGC1-alpha-R | GTGTGAGGAGGGTCATCGTT |
| Rno-NRF1-F | TTGATGGACACTTGGGTAGC |
| Rno-NRF1-R | GCCAGAAGGACTGAAAGCAG |
| Rno-TFAM-F | CAGAGTTGTCATTGGGATTGG |
| Rno-TFAM-R | TTCAGTGGGCAGAAGTCCAT |
| Rno-GLUT4-F | GTATGTTGCGGATGCTATGG |
| Rno-GLUT4-R | CCTCTGGTTTCAGGCACTCT |
| Rno-UCP3-F | CACGGATGTGGTGAAGGTC |
| Rno-UCP3-R | CTGGCGATGGTTCTGTAGG |
| Rno-NRF2B-F | GCCGTTTAGTCTCCGTGAAC |
| Rno-NRF2B-R | TCGTACTCCCAAGGCTGTGT |
| Rno-NRF2A-F | AGCAGAAGCACATCTCGTTG |
| Rno-NRF2A-R | CCCATCTCGTCACTTGCTCT |
| Rno-TFB1M-F | AAGGAAGTGGCGGAGAGAC |
| Rno-TFB1M-R | GGGATTGTAAAGAGGTGCTC |
| Rno-TFB2M-F | TGCGGATGGAGAGTTACAAG |
| Rno-TFB2M-R | ACACCTGCTGACCAAGGAAC |
| Rno-POLG-F | CTCCTACCTGCCTGTCAACC |
| Rno-POLG-R | GCTCCATCAGCGACTTCTTC |
| Rno-TOP1MT-F | CCAAGGTGTTTCGGACCTAC |
| Rno-TOP1MT-R | GTTTGCCCGGTTGTAAGCTA |
| Rno-SSBP1-F | AGCCAGCAGTTTGGTTCTTG |
| Rno-SSBP1-R | ATCGCCACATCTCATTTGTT |
| Rno-TWINKLE-F | GAGGACAGGGAGGAGGTCTT |
| Rno-TWINKLE-R | TGGTAAGGCCAAACATCACA |
| Rno-16SRNA-F | GACCCTGCTTGTAGCTGACC |
| Rno-16SRNA-R | ACCCTGATCCTTGAGACTGG |
| Rno-TBP2-F | TGTGAATACTGGTGCTGAG |
| Rno-TBP2-F | GGCATGAGACAAGACCTATA |

### REFERENCES

1. Wang, K. et al. A circular RNA protects the heart from pathological hypertrophy and heart failure by targeting miR-223. Eur Heart J 37, 2602-11 (2016).
2. Devaux, Y. et al. Circular RNAs in heart failure. Eur J Heart Fail (2017).
3. Jeck, W. R. & Sharpless, N. E. Detecting and characterizing circular RNAs. Nature Biotechnology 32, 453-461 (2014).
4. Nigro, J. M. et al. Scrambled exons. Cell 64, 607-13 (1991).
5. Salzman, J., Gawad, C., Wang, P. L., Lacayo, N. & Brown, P. O. Circular RNAs are the predominant transcript isoform from hundreds of human genes in diverse cell types. PLOS One 7, e30733 (2012).
6. Rybak-Wolf, A. et al. Circular RNAs in the Mammalian Brain Are Highly Abundant, Conserved, and Dynamically Expressed. Mol Cell 58, 870-85 (2015).
7. Westholm, J. O. et al. Genome-wide analysis of drosophila circular RNAs reveals their structural and sequence properties and age-dependent neural accumulation. Cell Rep 9, 1966-80 (2014).
8. Memczak, S. et al. Circular RNAs are a large class of animal RNAs with regulatory potency. Nature 495, 333-8 (2013).
9. Zheng, Q. P. et al. Circular RNA profiling reveals an abundant circHIPK3 that regulates cell growth by sponging multiple miRNAs. Nature Communications 7 (2016).
10. Du, W. W. et al. Foxo3 circular RNA retards cell cycle progression via forming ternary complexes with p21 and CDK2. Nucleic Acids Res 44, 2846-58 (2016).
11. Legnini, I. et al. Circ-ZNF609 Is a Circular RNA that Can Be Translated and Functions in Myogenesis. Mol Cell (2017).
12. Gupta, S. K. et al. Quaking Inhibits Doxorubicin-Mediated Cardiotoxicity Through Regulation of Cardiac Circular RNA Expression. Circ Res 122, 246-254 (2018).
13. Santer, L., Bar, C. & Thum, T. Circular RNAs: A Novel Class of Functional RNA Molecules with a Therapeutic Perspective. Mol Ther 27, 1350-1363 (2019).
14. Ucar, A. et al. The miRNA-212/132 family regulates both cardiac hypertrophy and cardiomyocyte autophagy. Nat Commun 3, 1078 (2012).
15. Chang, T. H. et al. An enhanced computational platform for investigating the roles of regulatory RNA and for identifying functional RNA motifs. BMC Bioinformatics 14 Suppl 2, S4 (2013).
16. Savage, K. I. et al. Identification of a BRCA1-mRNA splicing complex required for efficient DNA repair and maintenance of genomic stability. Mol Cell 54, 445-59 (2014).
17. Stylianidis, V., Hermans, K. C. M. & Blankesteijn, W. M. Wnt Signaling in Cardiac Remodeling and Heart Failure. Handb Exp Pharmacol 243, 371-393 (2017).
18. Mootha, V. K. et al. PGC-1alpha-responsive genes involved in oxidative phosphorylation are coordinately downregulated in human diabetes. Nat Genet 34, 267-73 (2003).
19. Subramanian, A. et al. Gene set enrichment analysis: a knowledge-based approach for interpreting genome-wide expression profiles. Proc Natl Acad Sci USA 102, 15545-50 (2005).
20. Bang, C. et al. Cardiac fibroblast-derived microRNA passenger strand-enriched exosomes mediate cardiomyocyte hypertrophy. J Clin Invest 124, 2136-46 (2014).
21. Bar, C., Chatterjee, S. & Thum, T. Long Noncoding RNAs in Cardiovascular Pathology, Diagnosis, and Therapy. Circulation 134, 1484-1499 (2016).
22. Viereck, J. & Thum, T. Circulating Noncoding RNAs as Biomarkers of Cardiovascular Disease and Injury. Circ Res 120, 381-399 (2017).
23. Ramunas, J. et al. Transient delivery of modified mRNA encoding TERT rapidly extends telomeres in human cells. FASEB J 29, 1930-9 (2015).

1. A pharmaceutical composition comprising a compound promoting the expression and/or the activity of the non-coding RNA (ncRNA) of SEQ ID NO: 1 or a sequence being at least 85% identical thereto.

2. A compound promoting the expression and/or the activity of the ncRNA of SEQ ID NO: 1 or a sequence being at least 85% identical thereto for use in treating or preventing a cardiac vascular disease or a metabolic disorder,
wherein the cardiac vascular disease is preferably heart failure or cardiac ischemia-reperfusion injury, and
wherein the heart failure is preferably anthracycline-induced heart failure, more preferably doxorubicin-, epirubicin- or daunorubicin-induced heart failure and most preferably doxorubicin-induced heart failure.

3. The pharmaceutical composition of claim 1 or the compound for use according to claim 2, wherein the compound is
(a) a nucleic acid sequence which comprises or consists of the nucleic acid sequence of the ncRNA of SEQ ID NO: 1 or a nucleic acid sequence which is at least 70% identical thereto,
(b) an expression vector expressing the nucleic acid sequence as defined in (a), preferably under the control of a heart-specific promoter, or
(c) a host comprising the expression vector of (b).

4. The pharmaceutical composition or the compound for use according to claim 3, wherein the nucleic acid sequence is a circular nucleic acid sequence.

5. The pharmaceutical composition or the compound for use according to claim 4, wherein the circular nucleic acid sequence is flanked by short interspersed nucleotide elements (SINEs) mediating the circularisation.

6. The pharmaceutical composition or the compound for use according to any one of claims 3 to 5, wherein the expression vector is an adeno-associated vector, preferably an AAV9 vector.

7. The pharmaceutical composition or the compound for use according to any one of claims 3 to 6, wherein the heart-specific promoter is a cardiomyocyte specific promoter, preferably the cardiomyocyte specific cTNT promoter.

8. The pharmaceutical composition of claim 1 or the compound for use of claim 2, wherein the compound as defined in (i) is
(a) a transcription factor promoting the expression of the ncRNA of SEQ ID NO: 1 or a sequence being at least 85% identical thereto, and/or
(b) a small molecule enhancing the expression of the ncRNA of SEQ ID NO: 1 or a sequence being at least 85% identical thereto.

9. A method for diagnosing heart failure or a predisposition to heart failure in a patient, comprising
(a) detecting the expression level of the ncRNA of SEQ ID NO: 1 or a sequence being at least 85% identical thereto in a sample obtained from the patient, and
(b) comparing the expression level of (a) with the expression level of the ncRNA of SEQ ID NO: 1 or a sequence being at least 85% identical thereto in a control sample obtained from healthy subjects or a predetermined standard from healthy subjects, wherein
a greater than 2-fold downregulation of the ncRNA of SEQ ID NO: 1 or a sequence being at least 85% identical thereto is indicative for a heart failure or a predisposition to heart failure in the patient.

10. The method of claim 10, wherein said samples are blood samples or blood-derived samples.

11. The method of claim 10, wherein said samples are a heart tissue samples.

12. The method of any one of claims 10 to 12, wherein healthy subjects are at least 3 healthy subjects, preferably at least 5 healthy subjects and most preferably at least 10 healthy subjects.

13. The method of any one of claims 10 to 13, wherein the detection of the expression level of the ncRNA comprises
(a) quantitative PCR, preferably quantitative real time PCR, or
(b) a template/RNA amplification method followed by determining the expression level of the ncRNA using a fluorescence- or luminescence-based quantification method.

14. The method of any one of claims 10 to 14, wherein the method comprises prior to the detection of the expression level of the ncRNA a pre-amplification step of the RNA within the test patient's sample and/or the control patient's sample.


SEQUENCE LISTING (91 sequences)

SEQ ID NO: 1 - RNA, 552 nt, Homo sapiens
  RNA sequence
ugugucccggcauggauauccggaacaaccucacuagguugcaugagcuggagaauugcucugucaucgaaggacacuugcagauacucuugauguucaaaacgaggcccgaagauuuccgagaccucaguuuccccaaacucaucaugaucacugauuacuugcugcucuuccgggucuaugggcucgagagccugaaggaccuguuccccaaccucacggucauccggggaucacgacuguucuuuaacuacgcgcuggucaucuucgagaugguucaccucaaggaacucggccucuacaaccugaugaacaucacccgggguucuguccgcaucgagaagaacaaugagcucuguuacuuggccacuaucgacuggucccguauccuggauuccguggaggauaauuacaucguguugaacaaagaugacaacgaggaguguggagacaucuguccggguaccgcgaagggcaagaccaacugccccgccaccgucaucaacgggcaguuugucgaacgauguuggacucauagucacugccagaaag

SEQ ID NO: 2 - RNA, 552 nt, Mus musculus
  RNA sequence
ugugcccugguauggacauccggaacaaccugaccaggcuacaugagcuggagaacugcucagucauugagggccaucugcagauccuccugauguucaagaccagacccgaagauuuccgagaccucaguuuccccaaacucaucaugaucacagauuaccugcuucucuuccgugucuauggucuggaaagucugaaagaccucuucccaaaucucacagucauccgaggcucccgucucuucuucaacuaugcccugguuaucuucgagaugguccaccugaaggagcuggggcuuuauaaccucaugaacaucacccggggcucuguccgcaucgagaagaauaaugagcucugcuaccuggccacuaucgacuggucccguauccuggauucuguggaggacaacuacauuguacugaacaaagaugacaacgaggaauguggggaugucuguccaggcaccgccaagggcaagaccaacuguccugccacugucaucaaugggcaguuuguggaacggugcuggacacacagucauugucagaaag

SEQ ID NO: 3 - RNA, 552 nt, Rattus norvegicus
  RNA sequence
ugugcccgggaauggacauccggaacaaccugaccagguugcaugagcuggagaacugcucggucauugagggccaucugcagauccucuugauguucaagaccagacccgaagauuuccgagaccucaguuuccccaaacucaucaugaucacagacuaccugcuucuuuuccgugucuauggccuagaaagucuaaaagaccucuucccaaaccucacagucauccgaggcucccgucucuucuucaacuaugcccuggucaucuucgagaugguccaccugaaggagcugggucuuuacaaccucaugaacaucacccggggcucuguccgcaucgagaagaauaaugaacucugcuaccuggccacuaucgacuggucccgcauccuggauuccguggaggacaacuacauuguacugaacaaagaugacaaugaggaauguggggacgucuguccaggcacagccaagggcaagaccaacugcccugccaccgucaucaaugggcaguuuguggaacggugcuggacacacagucauugucagaaag

SEQ ID NO: 4 - DNA, 306 bp, Homo sapiens
  ALU repeat upstream
ttatctttttttttttttttttttgagaaggagtttccctctggttgcccagggtggagtgcaatggcacaatctcggctcactgcaacctccgcctcccgggttcaagtgattctcctgcctcagcctcccaagtagctgggactacaggcgtgcaccgccacgcccggctaattttgtatttttagtagagatggggtttcaccgtgttggtcaggttggtctcgaactcctgacctcaagtggtccacccgcctcagcctccccaagtgctgggattacaggcctgagccaccgtgcccggcc

SEQ ID NO: 5 - DNA, 80 bp, Homo sapiens
  ALU repeat downstream
gaagaagaagtgaaacaactcagggcaagcactcaaacagtgcctgagacatagtaagtgctgcactaatgtcagctatt

SEQ ID NO: 6 - DNA, 966 bp, Homo sapiens
  circINSR left (upstream) flanking sequence (circular exon2)
ttatctttttttttttttttttttgagaaggagtttccctctggttgcccagggtggagtgcaatggcacaatctcggctcactgcaacctccgcctcccgggttcaagtgattctcctgcctcagcctcccaagtagctgggactacaggcgtgcaccgccacgcccggctaattttgtatttttagtagagatggggtttcaccgtgttggtcaggttggtctcgaactcctgacctcaagtggtccacccgcctcagcctccccaagtgctgggattacaggcctgagccaccgtgcccggcctggaaatcatcttaatgcccaataacaggaatggttaaggaaaccttcagtagacacgacagattcctgaagatcactagaaggaacaacccccaataaactactctcggcgacatgcgttgtggaataagatgaatgttttgcttttttttcgtttacaagtgtctttttccaaatgtctattataagcaatatcacttctataatcagataagaactaaactatttttttaaaaaaaccgaatctacagttttccactccttttcctctcccaagcaagaagaacattcactgactagcacaggcggtggcaaactgctgccctcgagcaaaatccggcccacgacttgtttttgtaaataaagttttattgaaacacagccacagtcatacatttatgcatgcctgtggctgcttgtccaatacagtggcagaattgagtcgttgcatcagagaccatatggcctgcaaaatctgaagtatctactatctgagtctttacagaaaatgtctgccactccctgggctagtgaatgccaccacccactattccccggcccctacctaatgaccatttaacatttttaagccataaaacattttaagctttctagaacaaggcacgagacactgcttagaaccctgtatccccggcgtac

SEQ ID NO: 7 - DNA, 830 bp, Homo sapiens
  circINSR right (downstream) flanking sequence (circular exon2)
ctacaagagaaaatgaacagaaagcaagacaggtgagcagacgcacggtggatgcatcagaaggatcaggggcagagccggcttcatggacaggaaacctgggccctgtgttttcatcccgggccctgtaaactctgcagccagccgggatgagtagctttctctgtaaagtacccatgcctaagtaattactttcaaaacgccaagaaaataattcttttggcaagccctgttaaaatccttaaaatcaccacctctgaaatgatgcagtcgggacaatgggattacaggggattgagtaacgcggcaggtgcagaaggagaaaggggaactgtctccagtgcgtcaacatagaagatcttcaggagataatttacggggataaaggtgcccgcggtcagaaatggggcaaggaaagctaaatgtatttgttttgtcaatcctcccacctcgctggctgactttccttcccggactgctctcctgacctgcaatcaagacctaattaccttttacctgggtgtggtgagggcttcccagggagcccgatctccattgcccaaatgccctgagttctcattctcctgaaggcggtccctcccaggccccaacgcccaccatggctccccatcatcctgcacttccctcaagattctgcccctacatcccctgcacaccactaaacaggcctgcccacaactcttgcacaactccaagactcctatcctcctctgtaacatggcttatatggaagaagaagtgaaacaactcagggcaagcactcaaacagtgcctgagacatagtaagtgctgcactaatgtcagctatt

SEQ ID NO: 8 - DNA, 552 bp, Homo sapiens
  DNA (genome) sequence
ctttctggcagtgactatgagtccaacatcgttcgacaaactgcccgttgatgacggtggcggggcagttggtcttgcccttcgcggtacccggacagatgtctccacactcctcgttgtcatctttgttcaacacgatgtaattatcctccacggaatccaggatacgggaccagtcgatagtggccaagtaacagagctcattgttcttctcgatgcggacagaaccccgggtgatgttcatcaggttgtagaggccgagttccttgaggtgaaccatctcgaagatgaccagcgcgtagttaaagaacagtcgtgatccccggatgaccgtgaggttggggaacaggtccttcaggctctcgagcccatagacccggaagagcagcaagtaatcagtgatcatgatgagtttggggaaactgaggtctcggaaatcttcgggcctcgttttgaacatcaagagtatctgcaagtgtccttcgatgacagagcaattctccagctcatgcaacctagtgaggttgttccggatatccatgccgggacaca

SEQ ID NO: 9 - DNA, 95 bp, Mus musculus
  ALU repeat upstream
acagtgtcctcctgtgacacaggttatctttaaactcataatcctcctgcctcagcctctttatcaatggagtttaagtggtcaccatgcctagc

SEQ ID NO: 10 - DNA, 145 bp, Mus musculus
  ALU repeat downstream
ccgggtgtggtggcgcacgcctttaatcccagcactcgggaggcagaggcaggtagatttctgagttctaggccagcctgttctacagagtgagtttcaggacagccagggctatatagtgaaaccctgtctcaaaaaaccaaaa

SEQ ID NO: 11 - DNA, 1188 bp, Mus musculus
  circINSR left (upstream) flanking sequence (circular exon2)
ctcactggggaattctatgaagggctatgtaccctcactgggagatctaggcaggggttctacagctgaacagtgtcctcctgtgacacaggttatctttaaactcataatcctcctgcctcagcctctttatcaatggagtttaagtggtcaccatgcctagcacatcatatatcattatctgtcatccacagttagggcaatggctggtatcataaagacaagaagtaacaaatactagtgagaatattaaggaaggggagcgtttacatactgttaacaaccaggaagattgaaaaggagtcctagttctttttcttcctcaagtcttttggttgacttatagacgaatgggtttcacgcgatgtcatcttatgtgcatcgatcgtaccttgtttatcacctctgctgcattttctgcactcagctctctgctcacccattagtatctttctttactcaactggtctctcttctgttcctgccgtgcacatatacagtgcatatatatctaattatttcattctttctggctactaccccattgtttctatatcccacatttttgatcttatctcttattattattactttcttgagacagggtctcaaatagcctaggcaggcatagtctagctcccacccattgaccgcttatttcataacttggttattgtgaagatgcctctatatgatatgtgccacattttcttcatctgtttttctgttggcagacatttcctcagctgtggtgaacagagcaacagtgagagtagatgtgcagtggagtgtctgtgatatgctgacctagagtgctctgggacatacacccctggttttttttttttttttttgtgtgtgtgtgagaagccaagaccaatttcaagggttagttgttcctcaggtgccctccatcttgtctcttgagacagggtctcttactggcctggagttcactaatagaagtagcccagtgagccctggggatccatctatctctaaatgccccctgccctcacagacatattatgttacacaagcttattacatgggtcctgggcatagatgctaggccctcatccctgtacaacactttgttgtctccccagttctaggttctagttccgttagccgttctagagtccctggctcacctactttgctttgcctttattatctgccttctag

SEQ ID NO: 12 - DNA, 736 bp, Mus musculus
  circINSR right (downstream) flanking sequence (circular exon2)
gtatgctgaaggcagtgccttttaaagatttctccgtggttagtatgcccaggagaagtagactcctgtaaaagtttaggtgatggaggttgtagggtacaaaccctagaatctccataaaaaatacattcgtccccttatctgcgaggcatacctatttgatgttgtacacttaactgtgggttactgagactgtaggaagcagaatttctgatggggagagatgaatgcgtgtactgatacttggccattgcagtaggaaggtagccacagttaggtatcaagatatggtgttttttttttaatgtggcttatatgtaaaaaaatttttatatgcaaaaaataaatagggggccagatttcttttgggggagagctgtttgccaccctctgtgctggatagtgactgtttctgtggtcctctgctttgttggggagtagactatgcaagagtatgggtttggcttagtgtttttcttatctgatcatagaagtcaatttgttagagatactcagaaaaagaaatgtaaaaattcagctttgaccgggtgtggtggcgcacgcctttaatcccagcactcgggaggcagaggcaggtagatttctgagttctaggccagcctgttctacagagtgagtttcaggacagccagggctatatagtgaaaccctgtctcaaaaaaccaaaaaaccaaaacaaacaaacaaaaaaaccccagctttgatgagggtagct

SEQ ID NO: 13 - DNA, 552 bp, Mus musculus
  DNA (genome) sequence
ctttctgacaatgactgtgtgtccagcaccgttccacaaactgcccattgatgacagtggcaggacagttggtcttgcccttggcggtgcctggacagacatccccacattcctcgttgtcatctttgttcagtacaatgtagttgtcctccacagaatccaggatacgggaccagtcgatagtggccaggtagcagagctcattattcttctcgatgcggacagagccccgggtgatgttcatgaggttataaagccccagctccttcaggtggaccatctcgaagataaccagggcatagttgaagaagagacgggagcctcggatgactgtgagatttgggaagaggtctttcagactttccagaccatagacacggaagagaagcaggtaatctgtgatcatgatgagtttggggaaactgaggtctcggaaatcttcgggtctggtcttgaacatcaggaggatctgcagatggccctcaatgactgagcagttctccagctcatgtagcctggtcaggttgttccggatgtccataccagggcaca

SEQ ID NO: 14 - DNA, 161 bp, Rattus norvegicus
  ALU repeat upstream
gggaacagagagatagctcagtgggtaaagacgcttgccaccagccagatcgcctccatttgatcctctagacccacacatgaggaaggagaattattctctaacctccatgttggtcatggcatccccatattcacatttataatacatagacacacaca

SEQ ID NO: 15 - DNA, 247 bp, Rattus norvegicus
  ALU repeat downstream
gagttggggagacaacaaagtgtttgttgtacaggtgtgagggcctgacatctatgcccaagaccatgtgataagctagtgcaacaaaatatgtctgtgagggctgcaggccttcagagacagatggatccccagggttcactgggtttcttctactagtgaactcctggccagtaagagaccctgtctcaagaaacaagatggggggcatgaggaaaaactgacccttgaaattggtcttggcctc

SEQ ID NO: 16 - DNA, 1557 bp, Rattus norvegicus
  circINSR left (upstream) flanking sequence (circular exon2)
gggaacagagagatagctcagtgggtaaagacgcttgccaccagccagatcgcctccatttgatcctctagacccacacatgaggaaggagaattattctctaacctccatgttggtcatggcatccccatattcacatttataatacatagacacacacatggagagataaatgatgttggtcataatgataaagatagatagattatagatcgatgatgggtagataggtaggtagataggtagatagatagatagctgataggtagatagatgataggtagatagatagatagatagatagatagatagatagctgataggtagattgatagatagatagataggtaggtagatagctgataggtagatagatagatagatagatagatagatagatagatagataatagttagatagctgataggtagatagataataggtagatagatgataggtaggtagggagagagagatagctgataggtagatagatgataggtagatagataggcagatagctgataagtagatagatagatgcataaatagatgatagatagctgatagatagatagatagatagatagctgatagatagatagatagatagatagatagatagatagatagatagataggtagatagatagatagctggtatatagatagatagaagcagatagatagatagatagatagatagatagatagatagatagatagatagctgataggtagatagatgatagatagatagatagatagatagatagatagatagatagataataggtagataggtagatagttgataggtagatagatagatgataggtagatagataggcagatagctgataggtaaatagatagatgcataaatagatgatggatagatagatagatagatagatagatagatagacagacagctggtagatagatagctgatagatagatagatagacagacagacagacagctggtaaatagatagctgatagatagatagatagatagatagatagatagatagatagatgaatttttacatttccttttctgagtatctcttacaaattgacttctatgatcagataagaaaagcatgctaagtcaaatccatactcctgcatagaccactcctcaatcaagcagaggaccacagagacatcactatccagcatagagggtggcaaacagctcccaaaagagtctgaccccctatttatttttcatatataaagtttttttacacatatgaacacatatgtaaaaaaaatcttgatagctaactgtggccacttgcctactacaatggccaagtttcagttccatcagagatttcactcctcacagtctcaataacccacagttaactgtacaatatccaatgggtatacctcacaggtagggggatgcatgtatttttatggagatcctagggtttggaccccacagccttcatcacctaaacgttcccaggagtctactgcccctgggtctactaaccatgaaggaatctttagaaggcgttgccaccttcaacatac

SEQ ID NO: 17 - DNA, 77 bp, Rattus norvegicus
  circINSR right (downstream) flanking sequence (circular exon2)
actagaggacagataatgaaagccaagcaaagtaggtcagccagggactctagagctgctaatggaactagagccta

SEQ ID NO: 18 - DNA, 552 bp, Rattus norvegicus
  DNA (genome) sequence
ctttctgacaatgactgtgtgtccagcaccgttccacaaactgcccattgatgacggtggcagggcagttggtcttgcccttggctgtgcctggacagacgtccccacattcctcattgtcatctttgttcagtacaatgtagttgtcctccacggaatccaggatgcgggaccagtcgatagtggccaggtagcagagttcattattcttctcgatgcggacagagccccgggtgatgttcatgaggttgtaaagacccagctccttcaggtggaccatctcgaagatgaccagggcatagttgaagaagagacgggagcctcggatgactgtgaggtttgggaagaggtcttttagactttctaggccatagacacggaaaagaagcaggtagtctgtgatcatgatgagtttggggaaactgaggtctcggaaatcttcgggtctggtcttgaacatcaagaggatctgcagatggccctcaatgaccgagcagttctccagctcatgcaacctggtcaggttgttccggatgtccattcccgggcaca

SEQ ID NO: 19 - RNA, 19 nt, Artificial Sequence
  Scramble siRNA
agguaguguaaucgccuug

SEQ ID NO: 20 - RNA, 19 nt, Artificial Sequence
  Circ-Insr siRNA1
gaaagugugcccugguaug

SEQ ID NO: 21 - RNA, 19 nt, Artificial Sequence
  Circ-Insr siRNA2
gucauugucagaaagugug

SEQ ID NO: 22 - DNA, 21 bp, Artificial Sequence
  Circ-Insr (mmu/rno/hsa) forward
ctgtcctgccactgtcatcaa

SEQ ID NO: 23 - DNA, 21 bp, Artificial Sequence
  Circ-Insr (mmu/rno/hsa) reverse
tcttcgggtctggtcttgaac

SEQ ID NO: 24 - DNA, 19 bp, Artificial Sequence
  Linear-Insr (mmu/rno/hsa) forward
tcacctgagtccctgaagg

SEQ ID NO: 25 - DNA, 20 bp, Artificial Sequence
  Linear-Insr (mmu/rno/hsa) reverse
gcatcaggtcagtgagtctc

SEQ ID NO: 26 - DNA, 23 bp, Artificial Sequence
  Pre-Insr (mmu/rno/hsa) forward
gatccatctatctctaaatgccc

SEQ ID NO: 27 - DNA, 21 bp, Artificial Sequence
  Pre-Insr (mmu/rno/hsa) reverse
tcttcgggtctggtcttgaac

SEQ ID NO: 28 - DNA, 20 bp, Artificial Sequence
  18s (mmu/rno) forward
gtaacccgttgaaccccatt

SEQ ID NO: 29 - DNA, 20 bp, Artificial Sequence
  18s (mmu/rno) reverse
ccatccaatcggtagtagcg

SEQ ID NO: 30 - DNA, 21 bp, Artificial Sequence
  18s (hsa) forward
agtccctgccctttgtacaca

SEQ ID NO: 31 - DNA, 21 bp, Artificial Sequence
  18s (hsa) reverse
gatccgagggcctcactaaac

SEQ ID NO: 32 - DNA, 18 bp, Artificial Sequence
  Xist (mmu) forward
tgcctggatttagaggag

SEQ ID NO: 33 - DNA, 20 bp, Artificial Sequence
  Xist (mmu) reverse
ctccacctagggatcgtcaa

SEQ ID NO: 34 - DNA, 19 bp, Artificial Sequence
  WNT6 (mmu) forward
gcaagactgggggttcgag

SEQ ID NO: 35 - DNA, 22 bp, Artificial Sequence
  WNT6 (mmu) reverse
cctgacaaccacactgtaggag

SEQ ID NO: 36 - DNA, 20 bp, Artificial Sequence
  Brca1 (mmu/rno) forward
atctgccgtccaaattcaag

SEQ ID NO: 37 - DNA, 21 bp, Artificial Sequence
  Brca1 (mmu/rno) reverse
ttccaaacagatcggacactc

SEQ ID NO: 38 - DNA, 23 bp, Artificial Sequence
  SSBP1 (mmu/rno) forward
caacaaatgagatgtggcgatca

SEQ ID NO: 39 - DNA, 22 bp, Artificial Sequence
  SSBP1 (mmu/rno) reverse
acgagcttcttaccagctatga

SEQ ID NO: 40 - DNA, 2476 bp, Artificial Sequence
  Circ-Insr overexpression transcript sequence
ctcactggggaattctatgaagggctatgtaccctcactgggagatctaggcaggggttctacagctgaacagtgtcctcctgtgacacaggttatctttaaactcataatcctcctgcctcagcctctttatcaatggagtttaagtggtcaccatgcctagcacatcatatatcattatctgtcatccacagttagggcaatggctggtatcataaagacaagaagtaacaaatactagtgagaatattaaggaaggggagcgtttacatactgttaacaaccaggaagattgaaaaggagtcctagttctttttcttcctcaagtcttttggttgacttatagacgaatgggtttcacgcgatgtcatcttatgtgcatcgatcgtaccttgtttatcacctctgctgcattttctgcactcagctctctgctcacccattagtatctttctttactcaactggtctctcttctgttcctgccgtgcacatatacagtgcatatatatctaattatttcattctttctggctactaccccattgtttctatatcccacatttttgatcttatctcttattattattactttcttgagacagggtctcaaatagcctaggcaggcatagtctagctcccacccattgaccgcttatttcataacttggttattgtgaagatgcctctatatgatatgtgccacattttcttcatctgtttttctgttggcagacatttcctcagctgtggtgaacagagcaacagtgagagtagatgtgcagtggagtgtctgtgatatgctgacctagagtgctctgggacatacacccctggttttttttttttttttttgtgtgtgtgtgagaagccaagaccaatttcaagggttagttgttcctcaggtgccctccatcttgtctcttgagacagggtctcttactggcctggagttcactaatagaagtagcccagtgagccctggggatccatctatctctaaatgccccctgccctcacagacatattatgttacacaagcttattacatgggtcctgggcatagatgctaggccctcatccctgtacaacactttgttgtctccccagttctaggttctagttccgttagccgttctagagtccctggctcacctactttgctttgcctttattatctgccttctagtgtgccctggtatggacatccggaacaacctgaccaggctacatgagctggagaactgctcagtcattgagggccatctgcagatcctcctgatgttcaagaccagacccgaagatttccgagacctcagtttccccaaactcatcatgatcacagattacctgcttctcttccgtgtctatggtctggaaagtctgaaagacctcttcccaaatctcacagtcatccgaggctcccgtctcttcttcaactatgccctggttatcttcgagatggtccacctgaaggagctggggctttataacctcatgaacatcacccggggctctgtccgcatcgagaagaataatgagctctgctacctggccactatcgactggtcccgtatcctggattctgtggaggacaactacattgtactgaacaaagatgacaacgaggaatgtggggatgtctgtccaggcaccgccaagggcaagaccaactgtcctgccactgtcatcaatgggcagtttgtggaacggtgctggacacacagtcattgtcagaaaggtatgctgaaggcagtgccttttaaagatttctccgtggttagtatgcccaggagaagtagactcctgtaaaagtttaggtgatggaggttgtagggtacaaaccctagaatctccataaaaaatacattcgtccccttatctgcgaggcatacctatttgatgttgtacacttaactgtgggttactgagactgtaggaagcagaatttctgatggggagagatgaatgcgtgtactgatacttggccattgcagtaggaaggtagccacagttaggtatcaagatatggtgttttttttttaatgtggcttatatgtaaaaaaatttttatatgcaaaaaataaatagggggccagatttcttttgggggagagctgtttgccaccctctgtgctggatagtgactgtttctgtggtcctctgctttgttggggagtagactatgcaagagtatgggtttggcttagtgtttttcttatctgatcatagaagtcaatttgttagagatactcagaaaaagaaatgtaaaaattcagctttgaccgggtgtggtggcgcacgcctttaatcccagcactcgggaggcagaggcaggtagatttctgagttctaggccagcctgttctacagagtgagtttcaggacagccagggctatatagtgaaaccctgtctcaaaaaaccaaaaaaccaaaacaaacaaacaaaaaaaccccagctttgatgagggtagct

SEQ ID NO: 41 - DNA, 23 bp, Artificial Sequence
  pcDNA3.1 plasmid system forward with EcorI
ctcactggggaattctatgaagg

SEQ ID NO: 42 - DNA, 30 bp, Artificial Sequence
  pcDNA3.1 plasmid system reverse with xhoI
ccgctcgagtagctaccctcatcaaagctg

SEQ ID NO: 43 - DNA, 23 bp, Artificial Sequence
  AAV plasmid system forward with EcorI
ctcactggggaattctatgaagg

SEQ ID NO: 44 - DNA, 29 bp, Artificial Sequence
  AAV plasmid system reverse with SalI
ccgtcgactagctaccctcatcaaagctg

SEQ ID NO: 45 - DNA, 23 bp, Artificial Sequence
  sgRNA1
acggaactagaacctagaactgg

SEQ ID NO: 46 - DNA, 23 bp, Artificial Sequence
  sgRNA2
gtcctgccactgtcatcaatggg

SEQ ID NO: 47 - DNA, 20 bp, Artificial Sequence
  Checking primer forward
cggggctggcttaagagttt

SEQ ID NO: 48 - DNA, 21 bp, Artificial Sequence
  Checking primer reverse
agtttgatcctctatgcccac

SEQ ID NO: 49 - DNA, 33 bp, Artificial Sequence
  Anti-linear-Insr probe
aaaattagacaggccttgataaggttgctcagc

SEQ ID NO: 50 - DNA, 33 bp, Artificial Sequence
  Anti-circ-Insr probe
aaaccataccagggcacactttctgacaatgac

SEQ ID NO: 51 - DNA, 39 bp, Artificial Sequence
  Back-splicing Site
gtcagtaacagtctttcacacgggcccttacctgtaggc

SEQ ID NO: 52 - DNA, 144 bp, Artificial Sequence
  Back splice site
ctggaccacagtcattgtcagaaagtgtgccctggtatggacatccggaacaacctgaccaggctacatgagctggagaactgctcagtcattgagggccatctgcagatcctcctgatgttcaagaccagacccgaagtaaaa

SEQ ID NO: 53 - DNA, 43 bp, Artificial Sequence
  sgRNA 1
gcttattacatgggtcctgggcatagatgctaggccctcatcc

SEQ ID NO: 54 - DNA, 34 bp, Artificial Sequence
  sgRNA 2
ccaactgtcctgccactgtcatcaatgggcagtt

SEQ ID NO: 55 - DNA, 23 bp, Artificial Sequence
  sgRNA 1
gcttattacatgggtcctgggca

SEQ ID NO: 56 - DNA, 10 bp, Artificial Sequence
  sgRNA 2
atgggcagtt

SEQ ID NO: 57 - DNA, 543 bp, Artificial Sequence
  SSBP1-Flag overexpression transcript sequence
gaattctctagagccaccatgtttcgaagacctgtgttacaggtatttcgtcagtttgtaagacatgagtctgaagtagccagcagtttggttcttgaacgatctctgaatcgtgttcagttacttggacgagtaggtcaggaccctgtcatgagacaggtggaaggaaaaaacccagtcacaatattttctctagcaacaaatgagatgtggcgatcaggggatagtgaagtataccaaatgggtgacgttagtcagaagacgacgtggcacagaatatcagtgtttcgaccaggcctcagagatgtggcatatcagtatgtgaaaaagggggctcgtatatttgtggaagggaaagtggactatggcgagtacatggataaaaacaatgtgaggcggcaagcaacaacaatcatagctgataacattatatttctgagtgaccagacaaaagaaaaggcagattacaaggatgacgacgataaggactataaggacgatgatgacaaggactacaaagatgatgacgataaatagggatcc

SEQ ID NO: 58 - DNA, 22 bp, Artificial Sequence
  Mt-CYTB forward primer
ggctacgtccttccatgaggac

SEQ ID NO: 59 - DNA, 24 bp, Artificial Sequence
  Mt-CYTB reverse primer
gaagccccctcaaattcattcgac

SEQ ID NO: 60 - DNA, 21 bp, Artificial Sequence
  ACTB forward primer
catctcctgctcgaagtctag

SEQ ID NO: 61 - DNA, 25 bp, Artificial Sequence
  ACTB reverse primer
atcatgtttgagaccttcaacaccc

SEQ ID NO: 62 - DNA, 21 bp, Artificial Sequence
  Rno-PGC1-alpha-F
ggagcaataaagcaaagagca

SEQ ID NO: 63 - DNA, 20 bp, Artificial Sequence
  Rno-PGC1-alpha-R
gtgtgaggagggtcatcgtt

SEQ ID NO: 64 - DNA, 20 bp, Artificial Sequence
  Rno-NRF1-F
ttgatggacacttgggtagc

SEQ ID NO: 65 - DNA, 20 bp, Artificial Sequence
  Rno-NRF1-R
gccagaaggactgaaagcag

SEQ ID NO: 66 - DNA, 21 bp, Artificial Sequence
  Rno-TFAM-F
cagagttgtcattgggattgg

SEQ ID NO: 67 - DNA, 20 bp, Artificial Sequence
  Rno-TFAM-R
ttcagtgggcagaagtccat

SEQ ID NO: 68 - DNA, 20 bp, Artificial Sequence
  Rno-GLUT4-F
gtatgttgcggatgctatgg

SEQ ID NO: 69 - DNA, 20 bp, Artificial Sequence
  Rno-GLUT4-R
cctctggtttcaggcactct

SEQ ID NO: 70 - DNA, 19 bp, Artificial Sequence
  Rno-UCP3-F
cacggatgtggtgaaggtc

SEQ ID NO: 71 - DNA, 19 bp, Artificial Sequence
  Rno-UCP3-R
ctggcgatggttctgtagg

SEQ ID NO: 72 - DNA, 20 bp, Artificial Sequence
  Rno-NRF2B-F
gccgtttagtctccgtgaac

SEQ ID NO: 73 - DNA, 20 bp, Artificial Sequence
  Rno-NRF2B-R
tcgtactcccaaggctgtgt

SEQ ID NO: 74 - DNA, 20 bp, Artificial Sequence
  Rno-NRF2A-F
agcagaagcacatctcgttg

SEQ ID NO: 75 - DNA, 20 bp, Artificial Sequence
  Rno-NRF2A-R
cccatctcgtcacttgctct

SEQ ID NO: 76 - DNA, 19 bp, Artificial Sequence
  Rno-TFB1M-F
aaggaagtggcggagagac

SEQ ID NO: 77 - DNA, 20 bp, Artificial Sequence
  Rno-TFB1M-R
gggattgtaaagaggtgctc

SEQ ID NO: 78 - DNA, 20 bp, Artificial Sequence
  Rno-TFB2M-F
tgcggatggagagttacaag

SEQ ID NO: 79 - DNA, 20 bp, Artificial Sequence
  Rno-TFB2M-R
acacctgctgaccaaggaac

SEQ ID NO: 80 - DNA, 20 bp, Artificial Sequence
  Rno-POLG-F
ctcctacctgcctgtcaacc

SEQ ID NO: 81 - DNA, 20 bp, Artificial Sequence
  Rno-POLG-R
gctccatcagcgacttcttc

SEQ ID NO: 82 - DNA, 20 bp, Artificial Sequence
  Rno-TOP1MT-F
ccaaggtgtttcggacctac

SEQ ID NO: 83 - DNA, 20 bp, Artificial Sequence
  Rno-TOP1MT-R
gtttgcccggttgtaagcta

SEQ ID NO: 84 - DNA, 20 bp, Artificial Sequence
  Rno-SSBP1-F
agccagcagtttggttcttg

SEQ ID NO: 85 - DNA, 20 bp, Artificial Sequence
  Rno-SSBP1-R
atcgccacatctcatttgtt

SEQ ID NO: 86 - DNA, 20 bp, Artificial Sequence
  Rno-TWINKLE-F
gaggacagggaggaggtctt

SEQ ID NO: 87 - DNA, 20 bp, Artificial Sequence
  Rno-TWINKLE-R
tggtaaggccaaacatcaca

SEQ ID NO: 88 - DNA, 20 bp, Artificial Sequence
  Rno-16SRNA-F
gaccctgcttgtagctgacc

SEQ ID NO: 89 - DNA, 20 bp, Artificial Sequence
  Rno-16SRNA-R
accctgatccttgagactgg

SEQ ID NO: 90 - DNA, 19 bp, Artificial Sequence
  Rno-TBP2-F
tgtgaatactggtgctgag

SEQ ID NO: 91 - DNA, 20 bp, Artificial Sequence
  Rno-TBP2-F
ggcatgagacaagacctata